(19) United States
(12) Patent Application Publication
(10) Pub. No.: US 2013/0259884 A1
(43) Pub. Date: Oct. 3, 2013

(54) PEPTIDES

(71) Applicants: Nigel Stuart Dunn-Coleman, El Sauzal (ES); Maria R. Diaz-Torres, El Sauzal (ES); Brian Miller, Manchester (GB)

(72) Inventors: Nigel Stuart Dunn-Coleman, El Sauzal (ES); Maria R. Diaz-Torres, El Sauzal (ES); Brian Miller, Manchester (GB)

(73) Assignee: ALERGENETICA SL, Santa Cruz, De Tenerife (ES)

(21) Appl. No.: 13/825,801

(22) PCT Filed: Sep. 23, 2011
(86) PCT No.: PCT/EP11/66610

§ 371 Date: Jun. 17, 2013

Related U.S. Application Data
(60) Provisional application No. 61/385,992, filed on Sep. 24, 2010.

(51) Int. Cl. C07K 7/08; C07K 7/06

(52) U.S. Cl. CPC C07K 7/08; C07K 7/06
USPC 424/185.1; 435/29; 435/325; 435/350; 435/351; 435/352; 435/353; 435/354; 435/366; 435/320.1; 530/326; 530/327; 530/328; 530/329; 536/23.1

(57) ABSTRACT

The application discloses peptides capable of preventing or treating fungal disease, including fungal allergy disease.

### FIELD OF THE INVENTION

The present invention relates to peptides capable of preventing or treating fungal disease and particularly, although not exclusively, to peptides useful in the prevention or treatment of fungal allergy disease.

### BACKGROUND TO THE INVENTION

Aspergillus and other infectious airborne fungi such as species of Alternaria (e.g. Alternaria alternata) are major causes of fungal allergy disease. For example, hypersensitivity to Aspergillus allergens may result in IgE-mediated asthma (2), allergic sinusitis or rhinitis or, in severe situations, ABPA. The allergens may be fungal proteins, but what determines which proteins are allergens is not known. Existing treatment of fungal allergy is through use of steroids or small-molecule anti-fungal agents. These are aimed at either relieving the symptoms of the allergy, e.g. allergic rhinitis, difficulty breathing, swelling, itchiness, or at reducing or controlling the extent of fungal infection. Fungal allergens can be grouped into several categories including proteases, glycosidases, components of protein production, oxidative stress response proteins and enzymes involved in gluconeogenesis or the pentose phosphate shunt (1).

In addition to acting as an allergen fungi such as Aspergillus and Alternaria are capable of active germination in the host leading to infection. This is in contrast to other allergens such as house dust mites, cat dander or grass pollen. Thus, fungal proteins can act as allergens and also cause airway damage through infection. They have also been reported to induce a ‘bystander-effect’ by enhancing the allergenic potential of other proteins (1).

### SAFS

Fungal infection of the lungs can lead to an allergic condition known as Severe Asthma with Fungal Sensitisation (SAFS) (1). This term was proposed by Denning et al (1) for patients having persistent severe or brittle asthma (despite treatment) and evidence of fungal sensitisation, as defined by positive prick testing, or fungus or fungal antigen-specific blood IgE testing, and who do not meet the criteria for ABPA.

Patients with SAFS have acute asthma with impaired pulmonary function. SAFS patients may present with nasal symptoms such as runny nose, sneezing and hay fever-like symptoms but do not produce plugs of sputum as seen in ABPA patients. CT scans may show mucous in the airways and eosinophilia is common.

SAFS diagnosis criteria are: (i) severe asthma (British Thoracic Society step 4 or worse), (ii) exclusion of ABPA (total IgE<1000 IU/mL), (iii) evidence of sensitisation to one or more fungi, by skin prick test or RAST test.

Some patients are sensitised to many fungi, but most only react to one or two fungi.

Common fungi that SAFS patients are sensitised to include: Aspergillus fumigatus, Candida albicans, Alternaria alternata, Trichopyton spp, Cladosporium herbarum, Penicillium chrysogenum and Botrytis cinerea.

Existing modes of treatment for patients with SAFS usually consist of multiple medication programmes. Inhaled and oral corticosteroids are used for long term control of the most severe symptoms but are associated with adverse events over the longer term, e.g. osteoporosis and weight gain. Short or long-acting beta-2 agonists or leukotriene antagonists may be used with some benefits.

Treatment with the anti-fungal itraconazole has some beneficial effect on pulmonary and nasal symptoms. Drug monitoring is required to optimise drug exposure and this may require switching between drug formulations and changes in the dosage amount.

### ABPA

Allergic Bronchopulmonary Aspergillosis (ABPA) is an allergic condition produced in response to Aspergillus. It is common in asthmatics. Symptoms are similar to those of asthma with intermittent episodes of coughing and wheezing. Some patients cough up brown plugs of mucus. Diagnosis can be made by X-ray or by sputum, skin and blood tests. In the longer term, and particularly if untreated, ABPA can lead to permanent lung damage through fibrosis.

Existing treatment is through use of steroids such as prednisolone by aerosol or oral administration but this has side-effects over the longer term as described above in respect of the use of steroids in SAFS treatment. Itraconazole can be used to reduce the amount of steroid required.

### Immunotherapy

Conventional immunotherapy for allergic disease has contemplated administration of whole protein allergens, but this often requires years of injections for successful desensitization and can lead to severe side-effects caused by IgE mediated allergic reactions, including anaphylaxis. Peptide based immunotherapy has been proposed as a vaccine treatment for cat allergy (3, 13). Failure to account for peptide MHC restriction has led to limited success (4).

A principal feature of MHC molecules is their allelic polymorphism, at least 707 class II molecules are known. MHC alleles have arisen under evolutionary pressure resulting in geographical diversity. Any poly-epitope vaccine targeting the whole population would need to bind a range of HLA molecules. MHC polymorphism thus greatly complicates epitope-based vaccine development, particularly in regard to population coverage (15).

Peptide-based allergy vaccines may be safer because they do not contain IgE-binding epitopes capable of crosslinking IgE. However, designing an effective peptide vaccine for human immunization is complicated by the polymorphism of the human MHC molecules and the antigenic complexity of allergens. The diversity of immunodominant sequences recognized in the context of the widely polymorphic HLA genotypes in man represents an obstacle for the development of synthetic peptides as potential diagnostic tools or vaccines (14). This points towards the need for a peptide cocktail in order to provide allergy protection for a majority of human patients (13).

### SUMMARY OF THE INVENTION

The inventors have identified peptides proposed to be useful in immunotherapy.

The peptides are preferably T-cell epitopes capable of binding human or animal HLA-DR molecules and stimulating an immune response. The peptides are preferably epitopes of fungal proteins. In particular, the peptides are preferably T-cell epitopes identified from Alternaria alternata proteins.

Modified peptides are also provided in which the wild type fungal peptide epitope amino acid sequence has been modified but still retains its ability to stimulate an immune response.

Accordingly, the present invention provides therapeutic compositions and methods for treating disease conditions in humans and animals associated with an antigen specific immune response by the human or animal to an antigen such as a protein antigen.

In accordance with the above, in one aspect of the present invention a peptide is provided, the peptide being chosen from one of:

| SEQ ID NO: | Peptide |
|---|---|
| 1-3 | FTTIASLFA |
| | MQFTTIASL |
| | IASLFAAAG |
| 4 | LAAAAPLES |
| 5 | WKISEFYGR |
| 6 | YYNSLGFNI |
| 7 | LGFNIKATN |
| 8 | FNIKATNGG |
| 9 | IKATNGGTL |
| 10 | ITYVATATL |
| 11 | YVATATLPN |
| 12 | LPNYCRAGG |
| 13 | LPNYVRAGG (*) |
| 14 | FVCQGVADA |
| 15 | FVVQGVADA (*) |
| 16 | YITLVTLPK |
| 17 | ITLVTLPKS |
| 18 | LFAAAGLAA |
| 19 | FAAAGLAAA |
| 20 | YNSLGFNIK |
| 21 | YCRAGGNGP |
| 22 | YVRAGGNGP (*) |
| 23 | LDFTCSAQA |
| 24 | LDFTVSAQA (*) |
| 25 | WYSCGENSF |
| 26 | WYSVGENSF (*) |
| 27 | VSDDITYVA |
| 28 | LVNHFTNEF |
| 29 | ILLLDVAPL |
| 30 | LRIINEPTA |
| 31 | LRRLRTACE |
| 32 | IQGFPTIKL |
| 33 | VVGLRSGQI |
| 34 | FVSTGTLGG |
| 35 | VVIVTGASG |
| 36 | LVITSSMSG |
| 37 | LVITSSLSG (*) |
| 38 | IVLLSLCQL |
| 39 | IVLLSLVQL (*) |
| 40 | FRDDRIEII |
| 41 | VAMNPVNTV |
| 42 | VALNPVNTV (*) |
| 43 | LVTIAKVDA |
| 44 | MKHLAAYLL |
| 45 | LKHLAAYLL(*) |
| 46 | YQKLKALAK |
| 47 | LKSCNALLL |
| 48 | LKSVNALLL (*) |
| 49 | WGVMVSHRS |
| 50 | WGVLVSHRS (*) |
| 51 | GYKTAFSML |
| 52 | GYKTAFSLL (*) |
| 53 | FVPQDIQKL |
| 54 | YVYFASDAS |
| 55 | YFIEPTIFS |
| 56 | YIQTKTVSI |
| 57 | IRLGDVLFG |
| 58 | WVNSYNTLH |

In the above, SEQ ID NOS: 13, 15, 22, 24, 26, 37, 39, 42, 45, 48, 50 and 52 (indicated by (*)) are Cys/Val and/or Met/Leu substitution variants of wild type SEQ ID NOS: 12, 14, 21, 23, 25, 36, 38, 41, 44, 47, 49 and 51, respectively. In some aspects, SEQ ID NOs: 13, 15, 22, 24, 26, 37, 39, 42, 45, 48, 50 and 52 are preferred compared to the respective corresponding sequence selected from one of SEQ ID NOs: 12, 14, 21, 23, 25, 36, 38, 41, 44, 47, 49 and 51.

In another aspect of the present invention a peptide is provided which peptide consists of or comprises the sequence of one of SEQ ID NOs:1-58. The amino acid sequence of the selected SEQ ID NO is preferably included in the peptide as a contiguous amino acid sequence.

In another aspect of the present invention a peptide is provided having at least 60% amino acid sequence identity to one of SEQ ID NOs:1-58. More preferably, the degree of sequence identity is one of 65%, 70%, 75%, 80%, 85%, 87%, 90%, 91%, 92%, 93%, 94%, 95%, 96%, 97%, 98%, 99% or 100% identity.

The minimum epitope for HLA DR recognition may be any of 7-11 amino acids in length and is typically a 9-mer epitope. Improved binding may be afforded by including at least one, two or three amino acids at one or both ends of the minimum epitope. Accordingly, peptides are provided as part of the present invention having a core amino acid sequence of any one of SEQ ID NOs:1-58 as well as an additional one, two, three, four, five, six (or more) amino acids of any type or combination at the N-terminal end, C-terminal end or at both the N- and C-terminal ends of the sequence. For example, a peptide may have a core amino acid sequence of any one of SEQ ID NOS:1-58 as well as an additional 1, 2, 3, 4, 5, 6, 7, 8, 9, 10, 11, 12, 13, 14, 15, amino acids at the N-terminal end, C-terminal end or at both the N- and C-terminal ends of the sequence.

The additional amino acids preferably correspond to amino acids from the parent protein amino acid sequence from which the peptide is derived, i.e. the wild-type amino acid sequence of the protein. For example, SEQ ID NOs:65, 112, 127, 142, 157, 172, 187, 202, 217, 255, 285, 300, 324, 359, 354, 384, 414, 444 and 459 are from the Alt a 1 protein (the position of the peptide in the Alt a 1 polypeptide is indicated in FIG. 21). In the parent protein (Alt a 1) the N-terminal Leucine of SEQ ID NO:112 is preceded by amino acids AAG and the C-terminal Serine is followed by amino acids RQD.

In some instances, addition of amino acids corresponding to those in the parent protein sequence in this way results in an unstable amino acid, e.g. cysteine (C), occurring at the N- and/or the C-terminal end of the peptide. In such cases, an unstable amino acid may be substituted by a more stable amino acid. For example, a CN and/or M/L substitution may be made (see, for example, SEQ ID NOs:217, 255, 285, 384, 414, 444, 609, 639, 654, 688, 718, 760, 790, 820, 850).

In one aspect of the present invention a peptide is provided, the peptide consisting of or comprising the amino acid sequence of one of SEQ ID NOs:1-913 or a peptide having a contiguous amino acid sequence having at least 70% sequence identity to the amino acid sequence of one of SEQ ID NOs: 1-913, wherein the peptide has an amino acid length of from 8 to 50 amino acids.

The degree of sequence identity may be chosen from one of 80%, 85%, 90% or 95%. The peptide may have a maximum length of 30 amino acids and a minimum length of 9 amino acids, or a maximum length of 20 amino acids and a minimum length of 11 amino acids, or a maximum length of 15 amino acids and a minimum length of 9 amino acids, or a maximum length of 11 amino acids and a minimum length of 8 amino acids, or a length of 9 or 15 amino acids. The peptide is preferably capable of stimulating an immune response.

In one aspect of the present invention a peptide is provided comprising the amino acid sequence of one of SEQ ID NOs:1-58 or a peptide having a contiguous amino acid sequence having at least 80% sequence identity to the amino acid sequence of one of SEQ ID NOs:1-58, wherein the peptide has an amino acid length of from 8 to 50 amino acids.

In one aspect of the present invention a peptide is provided, being chosen from one of:
(i) SEQ ID NO: 1, SEQ ID NOs: 59-80; or
(ii) SEQ ID NO: 2, SEQ ID NOs: 81-92; or
(iii) SEQ ID NO: 3, SEQ ID NOs: 93-105; or
(iv) SEQ ID NO: 4, SEQ ID NOs: 106-120; or
(v) SEQ ID NO: 5, SEQ ID NOs: 121-135; or
(vi) SEQ ID NO: 6, SEQ ID NOs: 136-150; or
(vii) SEQ ID NO: 7, SEQ ID NOs: 151-165; or
(viii) SEQ ID NO: 8, SEQ ID NOs: 166-180; or
(ix) SEQ ID NO: 9, SEQ ID NOs: 181-195; or
(x) SEQ ID NO: 10, SEQ ID NOs: 196-210; or
(xi) SEQ ID NO: 11, SEQ ID NOs: 211-233; or
(xii) SEQ ID NO: 12, SEQ ID NOs: 234-248; or
(xiii) SEQ ID NO: 13, SEQ ID NOs: 249-263; or
(xiv) SEQ ID NO: 14, SEQ ID NOs: 264-278; or
(xv) SEQ ID NO: 15, SEQ ID NOs: 279-293; or
(xvi) SEQ ID NO: 16, SEQ ID NOs: 294-307; or
(xvii) SEQ ID NO: 17, SEQ ID NOs: 308-317; or
(xviii) SEQ ID NO: 18, SEQ ID NOs: 318-332; or
(xix) SEQ ID NO: 19, SEQ ID NOs: 333-347; or
(xx) SEQ ID NO: 20, SEQ ID NOs: 348-362; or
(xxi) SEQ ID NO: 21, SEQ ID NOs: 363-377; or
(xxii) SEQ ID NO: 22, SEQ ID NOs: 378-392; or
(xxiii) SEQ ID NO: 23, SEQ ID NOs: 393-407; or
(xxiv) SEQ ID NO: 24, SEQ ID NOs: 408-422; or
(xxv) SEQ ID NO: 25, SEQ ID NOs: 423-437; or
(xxvi) SEQ ID NO: 26, SEQ ID NOs: 438-452; or
(xxvii) SEQ ID NO: 27, SEQ ID NOs: 453-467; or
(xxviii) SEQ ID NO: 28, SEQ ID NOs: 468-482; or
(xxix) SEQ ID NO: 29, SEQ ID NOs: 483-497; or
(xxx) SEQ ID NO: 30, SEQ ID NOs: 498-512; or
(xxxi) SEQ ID NO: 31, SEQ ID NOs: 513-527; or
(xxxii) SEQ ID NO: 32, SEQ ID NOs: 528-542; or
(xxxiii) SEQ ID NO: 33, SEQ ID NOs: 543-557; or
(xxxiv) SEQ ID NO: 34, SEQ ID NOs: 558-572; or
(xxxv) SEQ ID NO: 35, SEQ ID NOs: 573-587; or
(xxxvi) SEQ ID NO: 36, SEQ ID NOs: 588-602; or
(xxxvii) SEQ ID NO: 37, SEQ ID NOs: 603-617; or
(xxxviii) SEQ ID NO: 38, SEQ ID NOs: 618-632; or
(xxxix) SEQ ID NO: 39, SEQ ID NOs: 633-647; or
(xl) SEQ ID NO: 40, SEQ ID NOs: 648-662; or
(xli) SEQ ID NO: 41, SEQ ID NOs: 667-681; or
(xlii) SEQ ID NO: 42, SEQ ID NOs: 682-696; or
(xliii) SEQ ID NO: 43, SEQ ID NOs: 697-711; or
(xliv) SEQ ID NO: 44, SEQ ID NOs: 712-717; or
(xlv) SEQ ID NO: 45, SEQ ID NOs: 718-723; or
(xlvi) SEQ ID NO: 46, SEQ ID NOs: 724-738; or
(xlvii) SEQ ID NO: 47, SEQ ID NOs: 739-753; or
(xlviii) SEQ ID NO: 48, SEQ ID NOs: 754-768; or
(xlix) SEQ ID NO: 49, SEQ ID NOs: 769-783; or
(l) SEQ ID NO: 50, SEQ ID NOs: 784-798; or
(li) SEQ ID NO: 51, SEQ ID NOs: 799-813; or
(lii) SEQ ID NO: 52, SEQ ID NOs: 814-828; or
(liii) SEQ ID NO: 53, SEQ ID NOs: 829-843; or
(liv) SEQ ID NO: 54, SEQ ID NOs: 844-862; or
(lv) SEQ ID NO: 55, SEQ ID NOs: 863-877; or
(lvi) SEQ ID NO: 56, SEQ ID NOs: 878-892; or
(lvii) SEQ ID NO: 57, SEQ ID NOs: 893-898; or
(lviii) SEQ ID NO: 58, SEQ ID NOs: 899-913.

In one aspect of the present invention a pharmaceutical composition is provided, the pharmaceutical composition comprising a peptide according to any of the aspects and embodiments described herein. The pharmaceutical composition may further comprise a pharmaceutically acceptable carrier, adjuvant or diluent. The pharmaceutical composition may be a vaccine.

In some aspects of the present invention the peptides and/or pharmaceutical compositions are provided for use in the prevention or treatment of disease. The disease may be an allergic disease. The disease may be chosen from fungal allergy, fungal asthma, fungal infection, SAFS, ABPA, or Aspergillosis. The disease may be an allergic disease caused by an Alternaria alternata protein allergen or by infection of tissue by Alternaria alternata.

In another aspect of the present invention a method of treating or preventing disease in a patient in need of treatment thereof is provided, the method comprising administering to the patient a therapeutically effective amount of a peptide or pharmaceutical composition according to any one of the aspects and embodiments described herein.

In another aspect of the present invention a method for the production of a pharmaceutical composition is provided, the method comprising providing a peptide according to any one of the aspects and embodiments described herein, and mixing the peptide with a pharmaceutically acceptable carrier, adjuvant or diluent.

In another aspect of the present invention a nucleic acid, preferably an isolated and/or purified nucleic acid, encoding a peptide according to any one of the aspects and embodiments described herein is provided. A cell is also provided, having integrated in its genome a nucleic acid encoding a peptide according to any one of the aspects and embodiments described herein operably linked to a transcription control nucleic acid sequence. A nucleic acid expression vector is also provided having a nucleic acid encoding a peptide according to any one of the aspects and embodiments described herein operably linked to a transcription control nucleic acid sequence, wherein the vector is configured for expression of a peptide according to any one of the aspects and embodiments described herein when transfected into a suitable cell. Accordingly, a cell transfected with the nucleic acid expression vector is also provided.

In another aspect of the present invention a method of identifying a peptide that is capable of stimulating an immune response is provided, the method comprising the steps of:
(i) providing a candidate peptide having a contiguous amino acid sequence having at least 70% sequence identity to the amino acid sequence of one of SEQ ID NOs:1-913, wherein the peptide has an amino acid length of from 8 to 50 amino acids, and
(ii) testing the ability of the candidate peptide to induce an immune response.

Step (i) may comprise providing a peptide having the sequence of one of SEQ ID NOs:1-913 and chemically modifying the structure of the peptide to provide the candidate peptide. Step (ii) may comprise contacting the candidate peptide with a population of T cells in vitro and assaying T cell proliferation. Step (ii) may comprise or further comprise monitoring for production of IL-4 and/or IFNγ.

### DESCRIPTION OF PREFERRED EMBODIMENTS

In aspects and embodiments of the present invention a peptide is provided, the peptide comprising or consisting of one of SEQ ID NOs:59-913 set out below. In the sequences shown below, the 9mer peptide of the corresponding sequence selected from one of SEQ ID NOs:1-58 is shown in bold.

| Peptide | SEQ ID NO: |
|---|---|
| LQFTTIASLFA | 59 |
| QFTTIASLFA | 60 |
| FTTIASLFAAAGL | 61 |
| FTTIASLFAAAG | 62 |
| FTTIASLFAAA | 63 |
| FTTIASLFAA | 64 |
| LQFTTIASLFAAAGL | 65 |
| QFTTIASLFAAAGL | 66 |
| QFTTIASLFAAAG | 67 |
| QFTTIASLFAAA | 68 |
| QFTTIASLFAA | 69 |
| LQFTTIASLFAAAG | 70 |
| QFTTIASLFAAAG | 71 |
| LQFTTIASLFAAA | 72 |
| QFTTIASLFAAA | 73 |
| LQFTTIASLFAA | 74 |
| QFTTIASLFAA | 75 |
| MQFTTIASLFA | 76 |
| MQFTTIASLFAAAGL | 77 |
| MQFTTIASLFAAAG | 78 |
| MQFTTIASLFAAA | 79 |
| MQFTTIASLFAA | 80 |

SEQ ID NOs:59-80 correspond to SEQ ID NO:1 in which one, two or three additional contiguous amino acids from the Alt a 1 protein sequence are optionally incorporated at the N-terminus, C-terminus and both N- and C-terminus. An M/L substitution has been made in SEQ ID NOs:59, 65, 70, 73, 74.

| Peptide | SEQ ID NO: |
|---|---|
| MQFTTIASLFAAAGL | 81 |
| MQFTTIASLFAAAG | 82 |
| MQFTTIASLFAAA | 83 |
| MQFTTIASLFAA | 84 |
| MQFTTIASLFA | 85 |
| MQFTTIASLF | 86 |
| LQFTTIASLFAAAGL | 87 |
| LQFTTIASLFAAAG | 88 |
| LQFTTIASLFAAA | 89 |
| LQFTTIASLFAA | 90 |
| LQFTTIASLFA | 91 |
| LQFTTIASLF | 92 |

SEQ ID NOs:81-92 correspond to SEQ ID NO:2 in which one, two, three, four, five or six additional contiguous amino acids from the Alt a 1 protein sequence are incorporated at the C-terminus. An M/L substitution has been made in SEQ ID NOs:87-92.

| Peptide | SEQ ID NO: |
|---|---|
| LQFTTIASLFAAAGL | 93 |
| LQFTTIASLFAAAG | 94 |
| QFTTIASLFAAAG | 95 |
| FTTIASLFAAAG | 96 |
| TTIASLFAAAG | 97 |
| TIASLFAAAG | 98 |
| QFTTIASLFAAAGL | 99 |
| FTTIASLFAAAGL | 100 |
| TTIASLFAAAGL | 101 |
| TIASLFAAAGL | 102 |
| IASLFAAAGL | 103 |
| MQFTTIASLFAAAGL | 104 |
| MQFTTIASLFAAAG | 105 |

SEQ ID NOs:93-105 correspond to SEQ ID NO:3 in which one, two, three, four or five additional contiguous amino acids from the Alt a 1 protein sequence are optionally incorporated at the N-terminus, and a single amino acid are optionally incorporated C-terminus and optionally at both the N- and C-terminus.

| Peptide | SEQ ID NO: |
|---|---|
| AAGLAAAAPLES | 106 |
| AGLAAAAPLES | 107 |
| GLAAAAPLES | 108 |
| LAAAAPLESRQD | 109 |
| LAAAAPLESRQ | 110 |
| LAAAAPLESR | 111 |
| AAGLAAAAPLESRQD | 112 |
| AGLAAAAPLESRQD | 113 |
| GLAAAAPLESRQD | 114 |
| AAGLAAAAPLESRQ | 115 |
| AGLAAAAPLESRQ | 116 |
| GLAAAAPLESRQ | 117 |
| AAGLAAAAPLESR | 118 |
| AGLAAAAPLESR | 119 |
| GLAAAAPLESR | 120 |

SEQ ID NOs:106-120 correspond to SEQ ID NO:4 in which one, two or three additional contiguous amino acids from the Alt a 1 protein sequence are optionally incorporated at the N-terminus, C-terminus and both N- and C-terminus.

| Peptide | SEQ ID NO: |
|---|---|
| DYVWKISEFYGR | 121 |
| YVWKISEFYGR | 122 |
| VWKISEFYGR | 123 |
| WKISEFYGRKPE | 124 |
| WKISEFYGRKP | 125 |
| WKISEFYGRK | 126 |
| DYVWKISEFYGRKPE | 127 |
| DYVWKISEFYGRKP | 128 |
| DYVWKISEFYGRK | 129 |
| YVWKISEFYGRKPE | 130 |
| YVWKISEFYGRKP | 131 |
| YVWKISEFYGRK | 132 |
| VWKISEFYGRKPE | 133 |
| VWKISEFYGRKP | 134 |
| VWKISEFYGRK | 135 |

SEQ ID NOs:121-135 correspond to SEQ ID NO:5 in which one, two or three additional contiguous amino acids from the Alt a 1 protein sequence are optionally incorporated at the N-terminus, C-terminus and both N- and C-terminus.

| Peptide | SEQ ID NO: |
|---|---|
| EGTYYNSLGFNI | 136 |
| GTYYNSLGFNI | 137 |
| TYYNSLGFNI | 138 |
| YYNSLGFNIKAT | 139 |
| YYNSLGFNIKA | 140 |
| YYNSLGFNIK | 141 |
| EGTYYNSLGFNIKAT | 142 |
| EGTYYNSLGFNIKA | 143 |
| EGTYYNSLGFNIK | 144 |
| GTYYNSLGFNIKAT | 145 |
| GTYYNSLGFNIKA | 146 |
| GTYYNSLGFNIK | 147 |
| TYYNSLGFNIKAT | 148 |
| TYYNSLGFNIKA | 149 |
| TYYNSLGFNIK | 150 |

SEQ ID NOs:136-150 correspond to SEQ ID NO:6 in which one, two or three additional contiguous amino acids from the Alt a 1 protein sequence are optionally incorporated at the N-terminus, C-terminus and both N- and C-terminus.

| Peptide | SEQ ID NO: |
|---|---|
| YNSLGFNIKATN | 151 |
| NSLGFNIKATN | 152 |
| SLGFNIKATN | 153 |
| LGFNIKATNGGT | 154 |
| LGFNIKATNGG | 155 |
| LGFNIKATNG | 156 |
| YNSLGFNIKATNGGT | 157 |
| YNSLGFNIKATNGG | 158 |
| YNSLGFNIKATNG | 159 |
| NSLGFNIKATNGGT | 160 |
| NSLGFNIKATNGG | 161 |
| NSLGFNIKATNG | 162 |
| SLGFNIKATNGGT | 163 |
| SLGFNIKATNGG | 164 |
| SLGFNIKATNG | 165 |

SEQ ID NOs:151-165 correspond to SEQ ID NO:7 in which one, two or three additional contiguous amino acids from the Alt a 1 protein sequence are optionally incorporated at the N-terminus, C-terminus and both N- and C-terminus.

| Peptide | SEQ ID NO: |
|---|---|
| SLGFNIKATNGG | 166 |
| LGFNIKATNGG | 167 |
| GFNIKATNGG | 168 |
| FNIKATNGGTLD | 169 |
| FNIKATNGGTL | 170 |
| FNIKATNGGT | 171 |
| SLGFNIKATNGGTLD | 172 |
| SLGFNIKATNGGTL | 173 |
| SLGFNIKATNGGT | 174 |
| LGFNIKATNGGTLD | 175 |
| LGFNIKATNGGTL | 176 |
| LGFNIKATNGGT | 177 |
| GFNIKATNGGTLD | 178 |
| GFNIKATNGGTL | 179 |
| GFNIKATNGGT | 180 |

SEQ ID NOs:166-180 correspond to SEQ ID NO:8 in which one, two or three additional contiguous amino acids from the Alt a 1 protein sequence are optionally incorporated at the N-terminus, C-terminus and both N- and C-terminus.

| Peptide | SEQ ID NO: |
|---|---|
| GFNIKATNGGTL | 181 |
| FNIKATNGGTL | 182 |
| NIKATNGGTL | 183 |
| IKATNGGTLDFT | 184 |
| IKATNGGTLDF | 185 |
| IKATNGGTLD | 186 |
| GFNIKATNGGTLDFT | 187 |
| GFNIKATNGGTLDF | 188 |
| GFNIKATNGGTLD | 189 |
| FNIKATNGGTLDFT | 190 |
| FNIKATNGGTLDF | 191 |
| FNIKATNGGTLD | 192 |
| NIKATNGGTLDFT | 193 |
| NIKATNGGTLDF | 194 |
| NIKATNGGTLD | 195 |

SEQ ID NOs:181-195 correspond to SEQ ID NO:9 in which one, two or three additional contiguous amino acids from the Alt a 1 protein sequence are optionally incorporated at the N-terminus, C-terminus and both N- and C-terminus.

| Peptide | SEQ ID NO: |
|---|---|
| SDDITYVATATL | 196 |
| DDITYVATATL | 197 |
| DITYVATATL | 198 |
| ITYVATATLPNY | 199 |
| ITYVATATLPN | 200 |
| ITYVATATLP | 201 |
| SDDITYVATATLPNY | 202 |
| SDDITYVATATLPN | 203 |
| SDDITYVATATLP | 204 |
| DDITYVATATLPNY | 205 |
| DDITYVATATLPN | 206 |
| DDITYVATATLP | 207 |
| DITYVATATLPNY | 208 |
| DITYVATATLPN | 209 |
| DITYVATATLP | 210 |

SEQ ID NOs:196-210 correspond to SEQ ID NO:10 in which one, two or three additional contiguous amino acids from the Alt a 1 protein sequence are optionally incorporated at the N-terminus, C-terminus and both N- and C-terminus.

| Peptide | SEQ ID NO: |
|---|---|
| DITYVATATLPN | 211 |
| ITYVATATLPN | 212 |
| TYVATATLPN | 213 |
| YVATATLPNYVR | 214 |
| YVATATLPNYV | 215 |
| YVATATLPNY | 216 |
| DITYVATATLPNYVR | 217 |
| DITYVATATLPNYV | 218 |
| DITYVATATLPNY | 219 |
| ITYVATATLPNYVR | 220 |
| ITYVATATLPNYV | 221 |
| ITYVATATLPNY | 222 |
| TYVATATLPNYVR | 223 |
| TYVATATLPNYV | 224 |
| TYVATATLPNY | 225 |
| YVATATLPNYCR | 226 |
| YVATATLPNYC | 227 |
| DITYVATATLPNYCR | 228 |
| DITYVATATLPNYC | 229 |
| ITYVATATLPNYCR | 230 |
| ITYVATATLPNYC | 231 |
| TYVATATLPNYCR | 232 |
| TYVATATLPNYC | 233 |

SEQ ID NOs:211-233 correspond to SEQ ID NO:11 in which one, two or three additional contiguous amino acids from the Alt a 1 protein sequence are optionally incorporated at the N-terminus, C-terminus and both N- and C-terminus. A CN substitution has been made in SEQ ID NOs:214, 215, 217, 218, 220, 221, 223, 224.

| Peptide | SEQ ID NO: |
|---|---|
| TATLPNYCRAGG | 234 |
| ATLPNYCRAGG | 235 |
| TLPNYCRAGG | 236 |
| LPNYCRAGGNGP | 237 |
| LPNYCRAGGNG | 238 |
| LPNYCRAGGN | 239 |
| TATLPNYCRAGGNGP | 240 |
| TATLPNYCRAGGNG | 241 |
| TATLPNYCRAGGN | 242 |
| ATLPNYCRAGGNGP | 243 |
| ATLPNYCRAGGNG | 244 |
| ATLPNYCRAGGN | 245 |
| TLPNYCRAGGNGP | 246 |
| TLPNYCRAGGNG | 247 |
| TLPNYCRAGGN | 248 |

SEQ ID NOs:234-248 correspond to SEQ ID NO:12 in which one, two or three additional contiguous amino acids from the Alt a 1 protein sequence are optionally incorporated at the N-terminus, C-terminus and both N- and C-terminus.

| Peptide | SEQ ID NO: |
|---|---|
| TATLPNYVRAGG | 249 |
| ATLPNYVRAGG | 250 |
| TLPNYVRAGG | 251 |
| LPNYVRAGGNGP | 252 |
| LPNYVRAGGNG | 253 |
| LPNYVRAGGN | 254 |
| TATLPNYVRAGGNGP | 255 |
| TATLPNYVRAGGNG | 256 |
| TATLPNYVRAGGN | 257 |
| ATLPNYVRAGGNGP | 258 |
| ATLPNYVRAGGNG | 259 |
| ATLPNYVRAGGN | 260 |
| TLPNYVRAGGNGP | 261 |
| TLPNYVRAGGNG | 262 |
| TLPNYVRAGGN | 263 |

SEQ ID NOs:249-263 correspond to SEQ ID NO:13 in which one, two or three additional contiguous amino acids from the Alt a 1 protein sequence are optionally incorporated at the N-terminus, C-terminus and both N- and C-terminus.

| Peptide | SEQ ID NO: |
|---|---|
| PKDFVCQGVADA | 264 |
| KDFVCQGVADA | 265 |
| DFVCQGVADA | 266 |
| FVCQGVADAYIT | 267 |
| FVCQGVADAYI | 268 |
| FVCQGVADAY | 269 |
| PKDFVCQGVADAYIT | 270 |
| PKDFVCQGVADAYI | 271 |
| PKDFVCQGVADAY | 272 |
| KDFVCQGVADAYIT | 273 |
| KDFVCQGVADAYI | 274 |
| KDFVCQGVADAY | 275 |
| DFVCQGVADAYIT | 276 |
| DFVCQGVADAYI | 277 |
| DFVCQGVADAY | 278 |

SEQ ID NOs:264-278 correspond to SEQ ID NO:14 in which one, two or three additional contiguous amino acids from the Alt a 1 protein sequence are optionally incorporated at the N-terminus, C-terminus and both N- and C-terminus.

| Peptide | SEQ ID NO: |
|---|---|
| PKDFVVQGVADA | 279 |
| KDFVVQGVADA | 280 |
| DFVVQGVADA | 281 |
| FVVQGVADAYIT | 282 |
| FVVQGVADAYI | 283 |
| FVVQGVADAY | 284 |
| PKDFVVQGVADAYIT | 285 |
| PKDFVVQGVADAYI | 286 |
| PKDFVVQGVADAY | 287 |
| KDFVVQGVADAYIT | 288 |
| KDFVVQGVADAYI | 289 |
| KDFVVQGVADAY | 290 |
| DFVVQGVADAYIT | 291 |
| DFVVQGVADAYI | 292 |
| DFVVQGVADAY | 293 |

SEQ ID NOs:279-293 correspond to SEQ ID NO:15 in which one, two or three additional contiguous amino acids from the Alt a 1 protein sequence are optionally incorporated at the N-terminus, C-terminus and both N- and C-terminus.

| Peptide | SEQ ID NO: |
|---|---|
| VADAYITLVTLPK | 294 |
| ADAYITLVTLPK | 295 |
| DAYITLVTLPK | 296 |
| AYITLVTLPK | 297 |
| YITLVTLPKSS | 298 |
| YITLVTLPKS | 299 |
| VADAYITLVTLPKSS | 300 |
| VADAYITLVTLPKS | 301 |
| ADAYITLVTLPKSS | 302 |
| ADAYITLVTLPKS | 303 |
| DAYITLVTLPKSS | 304 |
| DAYITLVTLPKS | 305 |
| AYITLVTLPKSS | 306 |
| AYITLVTLPKS | 307 |

SEQ ID NOs:294-307 correspond to SEQ ID NO:16 in which one, two or three additional contiguous amino acids from the Alt a 1 protein sequence are optionally incorporated at the N-terminus, C-terminus and both N- and C-terminus.

| Peptide | SEQ ID NO: |
|---|---|
| VADAYITLVTLPKS | 308 |
| ADAYITLVTLPKS | 309 |
| DAYITLVTLPKS | 310 |
| AYITLVTLPKS | 311 |
| YITLVTLPKS | 312 |
| VADAYITLVTLPKSS | 313 |
| ADAYITLVTLPKSS | 314 |
| DAYITLVTLPKSS | 315 |
| AYITLVTLPKSS | 316 |
| YITLVTLPKSS | 317 |

SEQ ID NOs:308-317 correspond to SEQ ID NO:17 in which one, two, three, four or five additional contiguous amino acids from the Alt a 1 protein sequence are optionally incorporated at the N-terminus, and a single amino acid is optionally incorporated at the C-terminus.

| Peptide | SEQ ID NO: |
|---|---|
| IASLFAAAGLAA | 318 |
| ASLFAAAGLAA | 319 |
| SLFAAAGLAA | 320 |
| LFAAAGLAAAAP | 321 |
| LFAAAGLAAAA | 322 |
| LFAAAGLAAA | 323 |
| IASLFAAAGLAAAAP | 324 |
| IASLFAAAGLAAAA | 325 |
| IASLFAAAGLAAA | 326 |
| ASLFAAAGLAAAAP | 327 |
| ASLFAAAGLAAAA | 328 |
| ASLFAAAGLAAA | 329 |
| SLFAAAGLAAAAP | 330 |
| SLFAAAGLAAAA | 331 |
| SLFAAAGLAAA | 332 |

SEQ ID NOs:318-332 correspond to SEQ ID NO:18 in which one, two or three additional contiguous amino acids from the Alt a 1 protein sequence are optionally incorporated at the N-terminus, C-terminus and both N- and C-terminus.

| Peptide | SEQ ID NO: |
|---|---|
| ASLFAAAGLAAA | 333 |
| SLFAAAGLAAA | 334 |
| LFAAAGLAAA | 335 |
| FAAAGLAAAAPL | 336 |
| FAAAGLAAAAP | 337 |
| FAAAGLAAAA | 338 |
| ASLFAAAGLAAAAPL | 339 |
| ASLFAAAGLAAAAP | 340 |
| ASLFAAAGLAAAA | 341 |
| SLFAAAGLAAAAPL | 342 |
| SLFAAAGLAAAAP | 343 |
| SLFAAAGLAAAA | 344 |
| LFAAAGLAAAAPL | 345 |
| LFAAAGLAAAAP | 346 |
| LFAAAGLAAAA | 347 |

SEQ ID NOs:333-347 correspond to SEQ ID NO:19 in which one, two or three additional contiguous amino acids from the Alt a 1 protein sequence are optionally incorporated at the N-terminus, C-terminus and both N- and C-terminus.

| Peptide | SEQ ID NO: |
|---|---|
| GTYYNSLGFNIK | 348 |
| TYYNSLGFNIK | 349 |
| YYNSLGFNIK | 350 |
| YNSLGFNIKATN | 351 |
| YNSLGFNIKAT | 352 |
| YNSLGFNIKA | 353 |
| GTYYNSLGFNIKATN | 354 |
| GTYYNSLGFNIKAT | 355 |
| GTYYNSLGFNIKA | 356 |
| TYYNSLGFNIKATN | 357 |
| TYYNSLGFNIKAT | 358 |
| TYYNSLGFNIKA | 359 |
| YYNSLGFNIKATN | 360 |
| YYNSLGFNIKAT | 361 |
| YYNSLGFNIKA | 362 |

SEQ ID NOs:348-362 correspond to SEQ ID NO:20 in which one, two or three additional contiguous amino acids from the Alt a 1 protein sequence are optionally incorporated at the N-terminus, C-terminus and both N- and C-terminus.

| Peptide | SEQ ID NO: |
|---|---|
| LPNYCRAGGNGP | 363 |
| PNYCRAGGNGP | 364 |
| NYCRAGGNGP | 365 |
| YCRAGGNGPKDF | 366 |
| YCRAGGNGPKD | 367 |
| YCRAGGNGPK | 368 |
| LPNYCRAGGNGPKDF | 369 |
| LPNYCRAGGNGPKD | 370 |
| LPNYCRAGGNGPK | 371 |
| PNYCRAGGNGPKDF | 372 |
| PNYCRAGGNGPKD | 373 |
| PNYCRAGGNGPK | 374 |
| NYCRAGGNGPKDF | 375 |
| NYCRAGGNGPKD | 376 |
| NYCRAGGNGPK | 377 |

SEQ ID NOs:363-377 correspond to SEQ ID NO:21 in which one, two or three additional contiguous amino acids from the Alt a 1 protein sequence are optionally incorporated at the N-terminus, C-terminus and both N- and C-terminus.

| Peptide | SEQ ID NO: |
|---|---|
| LPNYVRAGGNGP | 378 |
| PNYVRAGGNGP | 379 |
| NYVRAGGNGP | 380 |
| YVRAGGNGPKDF | 381 |
| YVRAGGNGPKD | 382 |
| YVRAGGNGPK | 383 |
| LPNYVRAGGNGPKDF | 384 |
| LPNYVRAGGNGPKD | 385 |
| LPNYVRAGGNGPK | 386 |
| PNYVRAGGNGPKDF | 387 |
| PNYVRAGGNGPKD | 388 |
| PNYVRAGGNGPK | 389 |
| NYVRAGGNGPKDF | 390 |
| NYVRAGGNGPKD | 391 |
| NYVRAGGNGPK | 392 |

SEQ ID NOs:378-392 correspond to SEQ ID NO:22 in which one, two or three additional contiguous amino acids from the Alt a 1 protein sequence are optionally incorporated at the N-terminus, C-terminus and both N- and C-terminus.

| Peptide | SEQ ID NO: |
|---|---|
| GGTLDFTCSAQA | 393 |
| GTLDFTVCSAQA | 394 |
| TLDFTCSAQA | 395 |
| LDFTCSAQADKL | 396 |
| LDFTCSAQADK | 397 |
| LDFTCSAQAD | 398 |
| GGTLDFTCSAQADKL | 399 |
| GGTLDFTCSAQADK | 400 |
| GGTLDFTCSAQAD | 401 |
| GTLDFTCSAQADKL | 402 |
| GTLDFTCSAQADK | 403 |
| GTLDFTCSAQAD | 404 |
| TLDFTCSAQADKL | 405 |
| TLDFTCSAQADK | 406 |
| TLDFTCSAQAD | 407 |

SEQ ID NOs:393-407 correspond to SEQ ID NO:23 in which one, two or three additional contiguous amino acids from the Alt a 1 protein sequence are optionally incorporated at the N-terminus, C-terminus and both N- and C-terminus.

| Peptide | SEQ ID NO: |
|---|---|
| GGTLDFTVSAQA | 408 |
| GTLDFTVSAQA | 409 |
| TLDFTVSAQA | 410 |
| LDFTVSAQADKL | 411 |
| LDFTVSAQADK | 412 |
| LDFTVSAQAD | 413 |
| GGTLDFTVSAQADKL | 414 |
| GGTLDFTVSAQADK | 415 |
| GGTLDFTVSAQAD | 416 |
| GTLDFTVSAQADKL | 417 |
| GTLDFTVSAQADK | 418 |
| GTLDFTVSAQAD | 419 |
| TLDFTVSAQADKL | 420 |
| TLDFTVSAQADK | 421 |
| TLDFTVSAQAD | 422 |

SEQ ID NOs:408-422 correspond to SEQ ID NO:24 in which one, two or three additional contiguous amino acids from the Alt a 1 protein sequence are optionally incorporated at the N-terminus, C-terminus and both N- and C-terminus.

| Peptide | SEQ ID NO: |
|---|---|
| DHKWYSCGENSF | 423 |
| HKWYSCGENSF | 424 |
| KWYSCGENSF | 425 |
| WYSCGENSFLDF | 426 |
| WYSCGENSFLD | 427 |
| WYSCGENSFL | 428 |
| DHKWYSCGENSFLDF | 429 |
| DHKWYSCGENSFLD | 430 |
| DHKWYSCGENSFL | 431 |
| HKWYSCGENSFLDF | 432 |
| HKWYSCGENSFLD | 433 |
| HKWYSCGENSFL | 434 |
| KWYSCGENSFLDF | 435 |
| KWYSCGENSFLD | 436 |
| KWYSCGENSFL | 437 |

SEQ ID NOs:423-437 correspond to SEQ ID NO:25 in which one, two or three additional contiguous amino acids from the Alt a 1 protein sequence are optionally incorporated at the N-terminus, C-terminus and both N- and C-terminus.

| Peptide | SEQ ID NO: |
|---|---|
| DHKWYSVGENSF | 438 |
| HKWYSVGENSF | 439 |
| KWYSVGENSF | 440 |
| WYSVGENSFLDF | 441 |
| WYSVGENSFLD | 442 |
| WYSVGENSFL | 443 |
| DHKWYSVGENSFLDF | 444 |
| DHKWYSVGENSFLD | 445 |
| DHKWYSVGENSFL | 446 |
| HKWYSVGENSFLDF | 447 |
| HKWYSVGENSFLD | 448 |
| HKWYSVGENSFL | 449 |
| KWYSVGENSFLDF | 450 |
| KWYSVGENSFLD | 451 |
| KWYSVGENSFL | 452 |

SEQ ID NOs:438-452 correspond to SEQ ID NO:26 in which one, two or three additional contiguous amino acids from the Alt a 1 protein sequence are optionally incorporated at the N-terminus, C-terminus and both N- and C-terminus.

| Peptide | SEQ ID NO: |
|---|---|
| KQKVSDDITYVA | 453 |
| QKVSDDITYVA | 454 |
| KVSDDITYVA | 455 |
| VSDDITYVATAT | 456 |
| VSDDITYVATA | 457 |
| VSDDITYVAT | 458 |
| KQKVSDDITYVATAT | 459 |
| KQKVSDDITYVATA | 460 |
| KQKVSDDITYVAT | 461 |
| QKVSDDITYVATAT | 462 |
| QKVSDDITYVATA | 463 |
| QKVSDDITYVAT | 464 |
| KVSDDITYVATAT | 465 |
| KVSDDITYVATA | 466 |
| KVSDDITYVAT | 467 |

SEQ ID NOs:453-467 correspond to SEQ ID NO:27 in which one, two or three additional contiguous amino acids from the Alt a 1 protein sequence are optionally incorporated at the N-terminus, C-terminus and both N- and C-terminus.

| Peptide | SEQ ID NO: |
|---|---|
| DNRLVNHFTNEF | 468 |
| NRLVNHFTNEF | 469 |
| RLVNHFTNEF | 470 |
| LVNHFTNEFKRK | 471 |
| LVNHFTNEFKR | 472 |
| LVNHFTNEFK | 473 |
| DNRLVNHFTNEFKRK | 474 |
| DNRLVNHFTNEFKR | 475 |
| DNRLVNHFTNEFK | 476 |
| NRLVNHFTNEFKRK | 477 |
| NRLVNHFTNEFKR | 478 |
| NRLVNHFTNEFK | 479 |
| RLVNHFTNEFKRK | 480 |
| RLVNHFTNEFKR | 481 |
| RLVNHFTNEFK | 482 |

SEQ ID NOs:468-482 correspond to SEQ ID NO:28 in which one, two or three additional contiguous amino acids from the Alt a 3 protein sequence are optionally incorporated at the N-terminus, C-terminus and both N- and C-terminus.

| Peptide | SEQ ID NO: |
|---|---|
| TSEILLLDVAPL | 483 |
| SEILLLDVAPL | 484 |
| EILLLDVAPL | 485 |
| ILLLDVAPLSIG | 486 |
| ILLLDVAPLSI | 487 |
| ILLLDVAPLS | 488 |
| TSEILLLDVAPLSIG | 489 |
| TSEILLLDVAPLSI | 490 |
| TSEILLLDVAPLS | 491 |
| SEILLLDVAPLSIG | 492 |
| SEILLLDVAPLSI | 493 |
| SEILLLDVAPLS | 494 |
| EILLLDVAPLSIG | 495 |
| EILLLDVAPLSI | 496 |
| EILLLDVAPLS | 497 |

SEQ ID NOs:483-497 correspond to SEQ ID NO:29 in which one, two or three additional contiguous amino acids from the Alt a 3 protein sequence are optionally incorporated at the N-terminus, C-terminus and both N- and C-terminus.

| Peptide | SEQ ID NO: |
|---|---|
| LNVLRIINEPTA | 498 |
| NVLRIINEPTA | 499 |
| VLRIINEPTA | 500 |
| LRIINEPTAAAI | 501 |
| LRIINEPTAAA | 502 |
| LRIINEPTAA | 503 |
| LNVLRIINEPTAAAI | 504 |
| LNVLRIINEPTAAA | 505 |
| LNVLRIINEPTAA | 506 |
| NVLRIINEPTAAAI | 507 |
| NVLRIINEPTAAA | 508 |
| NVLRIINEPTAA | 509 |
| VLRIINEPTAAAI | 510 |
| VLRIINEPTAAA | 511 |
| VLRIINEPTAA | 512 |

SEQ ID NOs:498-512 correspond to SEQ ID NO: 30 in which one, two or three additional contiguous amino acids from the Alt a 3 protein sequence are optionally incorporated at the N-terminus, C-terminus and both N- and C-terminus.

| Peptide | SEQ ID NO: |
|---|---|
| ARALRRLRTACE | 513 |
| RALRRLRTACE | 514 |
| ALRRLRTACE | 515 |
| LRRLRTACERAK | 516 |
| LRRLRTACERA | 517 |
| LRRLRTACER | 518 |
| ARALRRLRTACERAK | 519 |
| ARALRRLRTACERA | 520 |
| ARALRRLRTACER | 521 |
| RALRRLRTACERAK | 522 |
| RALRRLRTACERA | 523 |
| RALRRLRTACER | 524 |
| ALRRLRTACERAK | 525 |
| ALRRLRTACERA | 526 |
| ALRRLRTACER | 527 |

SEQ ID NOs:513-527 correspond to SEQ ID NO:31 in which one, two or three additional contiguous amino acids from the Alt a 3 protein sequence are optionally incorporated at the N-terminus, C-terminus and both N- and C-terminus.

SEQ ID NOs:528-542 correspond to SEQ ID NO:32 in which one, two or three additional contiguous amino acids from the Alt a 4 protein sequence are optionally incorporated at the N-terminus, C-terminus and both N- and C-terminus.

| Peptide | SEQ ID NO: |
|---|---|
| ADIVVGLRSGQI | 543 |
| DIVVGLRSGQI | 544 |
| IVVGLRSGQI | 545 |
| VVGLRSGQIKTG | 546 |
| VVGLRSGQIKT | 547 |
| VVGLRSGQIK | 548 |
| ADIVVGLRSGQIKTG | 549 |
| ADIVVGLRSGQIKT | 550 |
| ADIVVGLRSGQIK | 551 |
| DIVVGLRSGQIKTG | 552 |
| DIVVGLRSGQIKT | 553 |
| DIVVGLRSGQIK | 554 |
| IVVGLRSGQIKTG | 555 |
| IVVGLRSGQIKT | 556 |
| IVVGLRSGQIK | 557 |

SEQ ID NOs:543-557 correspond to SEQ ID NO:33 in which one, two or three additional contiguous amino acids from the Alt a 6 protein sequence are optionally incorporated at the N-terminus, C-terminus and both N- and C-terminus.

| Peptide | SEQ ID NO: |
|---|---|
| PDEIQGFPTIKL | 528 |
| DEIQGFPTIKL | 529 |
| EIQGFPTIKL | 530 |
| IQGFPTIKLFPA | 531 |
| IQGFPTIKLFP | 532 |
| IQGFPTIKLF | 533 |
| PDEIQGFPTIKLFPA | 534 |
| PDEIQGFPTIKLFP | 535 |
| PDEIQGFPTIKLF | 536 |
| DEIQGFPTIKLFPA | 537 |
| DEIQGFPTIKLFP | 538 |
| DEIQGFPTIKLF | 539 |
| EIQGFPTIKLFPA | 540 |
| EIQGFPTIKLFP | 541 |
| EIQGFPTIKLF | 542 |

| Peptide | SEQ ID NO: |
|---|---|
| AGVFVSTGTLGG | 558 |
| GVFVSTGTLGG | 559 |
| VFVSTGTLGG | 560 |
| FVSTGTLGGGQE | 561 |
| FVSTGTLGGGQ | 562 |
| FVSTGTLGGG | 563 |
| AGVFVSTGTLGGGQE | 564 |
| AGVFVSTGTLGGGQ | 565 |
| AGVFVSTGTLGGG | 566 |
| GVFVSTGTLGGGQE | 567 |
| GVFVSTGTLGGGQ | 568 |
| GVFVSTGTLGGG | 569 |
| VFVSTGTLGGGQE | 570 |
| VFVSTGTLGGGQ | 571 |
| VFVSTGTLGGG | 572 |

SEQ ID NOs:558-572 correspond to SEQ ID NO:34 in which one, two or three additional contiguous amino acids from the Alt a 7 protein sequence are optionally incorporated at the N-terminus, C-terminus and both N- and C-terminus.

| Peptide | SEQ ID NO: |
|---|---|
| KGKVVIVTGASG | 573 |
| GKVVIVTGASG | 574 |
| KVVIVTGASG | 575 |
| VVIVTGASGPTG | 576 |
| VVIVTGASGPT | 577 |
| VVIVTGASGP | 578 |
| KGKVVIVTGASGPTG | 579 |
| KGKVVIVTGASGPT | 580 |
| KGKVVIVTGASGP | 581 |
| GKVVIVTGASGPTG | 582 |
| GKVVIVTGASGPT | 583 |
| GKVVIVTGASGP | 584 |
| KVVIVTGASGPTG | 585 |
| KVVIVTGASGPT | 586 |
| KVVIVTGASGP | 587 |

SEQ ID NOs:573-587 correspond to SEQ ID NO:35 in which one, two or three additional contiguous amino acids from the Alt a 8 protein sequence are optionally incorporated at the N-terminus, C-terminus and both N- and C-terminus.

| Peptide | SEQ ID NO: |
|---|---|
| TGSLVITSSMSG | 588 |
| GSLVITSSMSG | 589 |
| SLVITSSMSG | 590 |
| LVITSSMSGHIA | 591 |
| LVITSSMSGHI | 592 |
| LVITSSMSGH | 593 |
| TGSLVITSSMSGHIA | 594 |
| TGSLVITSSMSGHI | 595 |
| TGSLVITSSMSGH | 596 |
| GSLVITSSMSGHIA | 597 |
| GSLVITSSMSGHI | 598 |
| GSLVITSSMSGH | 599 |
| SLVITSSMSGHIA | 600 |
| SLVITSSMSGHI | 601 |
| SLVITSSMSGH | 602 |

SEQ ID NOs:588-602 correspond to SEQ ID NO:36 in which one, two or three additional contiguous amino acids from the Alt a 8 protein sequence are optionally incorporated at the N-terminus C-terminus and both N- and C-terminus.

| Peptide | SEQ ID NO: |
|---|---|
| TGSLVITSSLSG | 603 |
| GSLVITSSLSG | 604 |
| SLVITSSLSG | 605 |
| LVITSSLSGHIA | 606 |
| LVITSSLSGHI | 607 |
| LVITSSLSGH | 608 |
| TGSLVITSSLSGHIA | 609 |
| TGSLVITSSLSGHI | 610 |
| TGSLVITSSLSGH | 611 |
| GSLVITSSLSGHIA | 612 |
| GSLVITSSLSGHI | 613 |
| GSLVITSSLSGH | 614 |
| SLVITSSLSGHIA | 615 |
| SLVITSSLSGHI | 616 |
| SLVITSSLSGH | 617 |

SEQ ID NOs:603-617 correspond to SEQ ID NO:37 in which one, two or three additional contiguous amino acids from the Alt a 8 protein sequence are optionally incorporated at the N-terminus, C-terminus and both N- and C-terminus.

| Peptide | SEQ ID NO: |
|---|---|
| VWAIVLLSLCQL | 618 |
| WAIVLLSLCQL | 619 |
| AIVLLSLCQL | 620 |
| IVLLSLCQLTTP | 621 |
| IVLLSLCQLTT | 622 |
| IVLLSLCQLT | 623 |
| VWAIVLLSLCQLTTP | 624 |
| VWAIVLLSLCQLTT | 625 |
| VWAIVLLSLCQLT | 626 |
| WAIVLLSLCQLTTP | 627 |
| WAIVLLSLCQLTT | 628 |
| WAIVLLSLCQLT | 629 |
| AIVLLSLCQLTTP | 630 |
| AIVLLSLCQLTT | 631 |
| AIVLLSLCQLT | 632 |

SEQ ID NOs:618-632 correspond to SEQ ID NO:38 in which one, two or three additional contiguous amino acids from the Alt a 10 protein sequence are optionally incorporated at the N-terminus, C-terminus and both N- and C-terminus.

| Peptide | SEQ ID NO: |
|---|---|
| VWAIVLLSLVQL | 633 |
| WAIVLLSLVQL | 634 |
| AIVLLSLVQL | 635 |
| IVLLSLVQLTTP | 636 |
| IVLLSLVQLTT | 637 |
| IVLLSLVQLT | 638 |
| VWAIVLLSLVQLTTP | 639 |
| VWAIVLLSLVQLTT | 640 |
| VWAIVLLSLVQLT | 641 |
| WAIVLLSLVQLTTP | 642 |
| WAIVLLSLVQLTT | 643 |
| WAIVLLSLVQLT | 644 |
| AIVLLSLVQLTTP | 645 |
| AIVLLSLVQLTT | 646 |
| AIVLLSLVQLT | 647 |

SEQ ID NOs:633-647 correspond to SEQ ID NO: 39 in which one, two or three additional contiguous amino acids from the Alt a 10 protein sequence are optionally incorporated at the N-terminus, C-terminus and both N- and C-terminus.

| Peptide | SEQ ID NO: |
|---|---|
| VGIFRDDRIEII | 648 |
| GIFRDDRIEII | 649 |
| IFRDDRIEII | 650 |
| FRDDRIEIIAND | 651 |
| FRDDRIEIIAN | 652 |
| FRDDRIEIIA | 653 |
| VGIFRDDRIEIIAND | 654 |
| VGIFRDDRIEIIAN | 655 |
| VGIFRDDRIEIIA | 656 |
| GIFRDDRIEIIAND | 657 |
| GIFRDDRIEIIAN | 658 |
| GIFRDDRIEIIA | 659 |
| IFRDDRIEIIAND | 660 |
| IFRDDRIEIIAN | 661 |
| IFRDDRIEIIA | 662 |

SEQ ID NOs:648-662 correspond to SEQ ID NO:40 in which one, two or three additional contiguous amino acids from the Alt a 3 protein sequence are optionally incorporated at the N-terminus, C-terminus and both N- and C-terminus.

| Peptide | SEQ ID NO: |
|---|---|
| KNQVAMNPVNTV | 667 |
| NQVAMNPVNTV | 668 |
| QVAMNPVNTV | 669 |
| VAMNPVNTVFDA | 670 |
| VAMNPVNTVFD | 671 |
| VAMNPVNTVF | 672 |
| KNQVAMNPVNTVFDA | 673 |
| KNQVAMNPVNTVFD | 674 |
| KNQVAMNPVNTVF | 675 |
| NQVAMNPVNTVFDA | 676 |
| NQVAMNPVNTVFD | 677 |
| NQVAMNPVNTVF | 678 |
| QVAMNPVNTVFDA | 679 |
| QVAMNPVNTVFD | 680 |
| QVAMNPVNTVF | 681 |

SEQ ID NOs:667-681 correspond to SEQ ID NO:41 in which one, two or three additional contiguous amino acids from the Alt a 3 protein sequence are optionally incorporated at the N-terminus, C-terminus and both N- and C-terminus.

| Peptide | SEQ ID NO: |
|---|---|
| KNQVALNPVNTV | 682 |
| NQVALNPVNTV | 683 |
| QVALNPVNTV | 684 |
| VALNPVNTVFDA | 685 |
| VALNPVNTVFD | 686 |
| VALNPVNTVF | 687 |
| KNQVALNPVNTVFDA | 688 |
| KNQVALNPVNTVFD | 689 |
| KNQVALNPVNTVF | 690 |
| NQVALNPVNTVFDA | 691 |
| NQVALNPVNTVFD | 692 |
| NQVALNPVNTVF | 693 |
| QVALNPVNTVFDA | 694 |
| QVALNPVNTVFD | 695 |
| QVALNPVNTVF | 696 |

SEQ ID NOs:682-696 correspond to SEQ ID NO:42 in which one, two or three additional contiguous amino acids from the Alt a 3 protein sequence are optionally incorporated at the N-terminus, C-terminus and both N- and C-terminus.

| Peptide | SEQ ID NO: |
|---|---|
| LSKLVTIAKVDA | 697 |
| SKLVTIAKVDA | 698 |
| KLVTIAKVDA | 699 |
| LVTIAKVDATLN | 700 |
| LVTIAKVDATL | 701 |
| LVTIAKVDAT | 702 |
| LSKLVTIAKVDATLN | 703 |
| LSKLVTIAKVDATL | 704 |
| LSKLVTIAKVDAT | 705 |
| SKLVTIAKVDATLN | 706 |
| SKLVTIAKVDATL | 707 |
| SKLVTIAKVDAT | 708 |
| KLVTIAKVDATLN | 709 |
| KLVTIAKVDATL | 710 |
| KLVTIAKVDAT | 711 |

SEQ ID NOs:697-711 correspond to SEQ ID NO: 43 in which one, two or three additional contiguous amino acids from the Alt a 4 protein sequence are optionally incorporated at the N-terminus, C-terminus and both N- and C-terminus.

| Peptide | SEQ ID NO: |
|---|---|
| MKHLAAYLLLGLGGN | 712 |
| MKHLAAYLLLGLGG | 713 |
| MKHLAAYLLLGLG | 714 |
| MKHLAAYLLLGL | 715 |
| MKHLAAYLLLG | 716 |
| MKHLAAYLLL | 717 |

SEQ ID NOs:712-717 correspond to SEQ ID NO:44 in which one, two, three, four, five or six additional contiguous amino acids from the Alt a 5 protein sequence are optionally incorporated at the C-terminus.

| Peptide | SEQ ID NO: |
|---|---|
| LKHLAAYLLLGLGGN | 718 |
| LKHLAAYLLLGLGG | 719 |
| LKHLAAYLLLGLG | 720 |
| LKHLAAYLLLGL | 721 |
| LKHLAAYLLLG | 722 |
| LKHLAAYLLL | 723 |

SEQ ID NOs:718-723 correspond to SEQ ID NO:45 in which one, two, three, four, five or six additional contiguous amino acids from the Alt a 5 protein sequence are optionally incorporated at the C-terminus.

| Peptide | SEQ ID NO: |
|---|---|
| AEVYQKLKALAK | 724 |
| EVYQKLKALAK | 725 |
| VYQKLKALAK | 726 |
| YQKLKALAKKTY | 727 |
| YQKLKALAKKT | 728 |
| YQKLKALAKK | 729 |
| AEVYQKLKALAKKTY | 730 |
| AEVYQKLKALAKKT | 731 |
| AEVYQKLKALAKK | 732 |
| EVYQKLKALAKKTY | 733 |
| EVYQKLKALAKKT | 734 |
| EVYQKLKALAKK | 735 |
| VYQKLKALAKKTY | 736 |
| VYQKLKALAKKT | 737 |
| VYQKLKALAKK | 738 |

SEQ ID NOs:724-738 correspond to SEQ ID NO:46 in which one, two or three additional contiguous amino acids from the Alt a 6 protein sequence are optionally incorporated at the N-terminus, C-terminus and both N- and C-terminus.

| Peptide | SEQ ID NO: |
|---|---|
| AIELKSCNALLL | 739 |
| IELKSCNALLL | 740 |
| ELKSCNALLL | 741 |
| LKSCNALLLKVN | 742 |
| LKSCNALLLKV | 743 |
| LKSCNALLLK | 744 |
| AIELKSCNALLLKVN | 745 |
| AIELKSCNALLLKV | 746 |
| AIELKSCNALLLK | 747 |
| IELKSCNALLLKVN | 748 |
| IELKSCNALLLKV | 749 |
| IELKSCNALLLK | 750 |
| ELKSCNALLLKVN | 751 |
| ELKSCNALLLKV | 752 |
| ELKSCNALLLK | 753 |

SEQ ID NOs:739-753 correspond to SEQ ID NO:47 in which one, two or three additional contiguous amino acids from the Alt a 6 protein sequence are optionally incorporated at the N-terminus, C-terminus and both N- and C-terminus.

| Peptide | SEQ ID NO: |
|---|---|
| AIELKSVNALLL | 754 |
| IELKSVNALLL | 755 |
| ELKSVNALLL | 756 |
| LKSVNALLLKVN | 757 |
| LKSVNALLLKV | 758 |
| LKSVNALLLK | 759 |
| AIELKSVNALLLKVN | 760 |
| AIELKSVNALLLKV | 761 |
| AIELKSVNALLLK | 762 |
| IELKSVNALLLKVN | 763 |
| IELKSVNALLLKV | 764 |
| IELKSVNALLLK | 765 |
| ELKSVNALLLKVN | 766 |
| ELKSVNALLLKV | 767 |
| ELKSVNALLLK | 768 |

SEQ ID NOs:754-768 correspond to SEQ ID NO:48 in which one, two or three additional contiguous amino acids from the Alt a 6 protein sequence are optionally incorporated at the N-terminus, C-terminus and both N- and C-terminus.

| Peptide | SEQ ID NO: |
|---|---|
| GAGWGVMVSHRS | 769 |
| AGWGVMVSHRS | 770 |
| GWGVMVSHRS | 771 |
| WGVMVSHRSGET | 772 |
| WGVMVSHRSGE | 773 |
| WGVMVSHRSG | 774 |
| GAGWGVMVSHRSGET | 775 |
| GAGWGVMVSHRSGE | 776 |
| GAGWGVMVSHRSG | 777 |
| AGWGVMVSHRSGET | 778 |
| AGWGVMVSHRSGE | 779 |
| AGWGVMVSHRSG | 780 |
| GWGVMVSHRSGET | 781 |
| GWGVMVSHRSGE | 782 |
| GWGVMVSHRSG | 783 |

SEQ ID NOs:769-783 correspond to SEQ ID NO:49 in which one, two or three additional contiguous amino acids from the Alt a 6 protein sequence are optionally incorporated at the N-terminus, C-terminus and both N- and C-terminus.

| Peptide | SEQ ID NO: |
|---|---|
| GAGWGVLVSHRS | 784 |
| AGWGVLVSHRS | 785 |
| GWGVLVSHRS | 786 |
| WGVLVSHRSGET | 787 |
| WGVLVSHRSGE | 788 |
| WGVLVSHRSG | 789 |
| GAGWGVLVSHRSGET | 790 |
| GAGWGVLVSHRSGE | 791 |
| GAGWGVLVSHRSG | 792 |
| AGWGVLVSHRSGET | 793 |
| AGWGVLVSHRSGE | 794 |
| AGWGVLVSHRSG | 795 |
| GWGVLVSHRSGET | 796 |
| GWGVLVSHRSGE | 797 |
| GWGVLVSHRSG | 798 |

SEQ ID NOs:784-798 correspond to SEQ ID NO:50 in which one, two or three additional contiguous amino acids from the Alt a 6 protein sequence are optionally incorporated at the N-terminus, C-terminus and both N- and C-terminus.

| Peptide | SEQ ID NO: |
|---|---|
| VPLGYKTAFSML | 799 |
| PLGYKTAFSML | 800 |
| LGYKTAFSML | 801 |
| GYKTAFSMLANL | 802 |
| GYKTAFSMLAN | 803 |
| GYKTAFSMLA | 804 |
| VPLGYKTAFSMLANL | 805 |
| VPLGYKTAFSMLAN | 806 |
| VPLGYKTAFSMLA | 807 |
| PLGYKTAFSMLANL | 808 |
| PLGYKTAFSMLAN | 809 |
| PLGYKTAFSMLA | 810 |
| LGYKTAFSMLANL | 811 |
| LGYKTAFSMLAN | 812 |
| LGYKTAFSMLA | 813 |

SEQ ID NOs:799-813 correspond to SEQ ID NO:51 in which one, two or three additional contiguous amino acids from the Alt a 7 protein sequence are optionally incorporated at the N-terminus, C-terminus and both N- and C-terminus.

| Peptide | SEQ ID NO: |
|---|---|
| VPLGYKTAFSLL | 814 |
| PLGYKTAFSLL | 815 |
| LGYKTAFSLL | 816 |
| GYKTAFSLLANL | 817 |
| GYKTAFSLLAN | 818 |
| GYKTAFSLLA | 819 |
| VPLGYKTAFSLLANL | 820 |
| VPLGYKTAFSLLAN | 821 |
| VPLGYKTAFSLLA | 822 |
| PLGYKTAFSLLANL | 823 |
| PLGYKTAFSLLAN | 824 |
| PLGYKTAFSLLA | 825 |
| LGYKTAFSLLANL | 826 |
| LGYKTAFSLLAN | 827 |
| LGYKTAFSLLA | 828 |

SEQ ID NOs:814-828 correspond to SEQ ID NO:52 in which one, two or three additional contiguous amino acids from the Alt a 7 protein sequence are optionally incorporated at the N-terminus, C-terminus and both N- and C-terminus.

| Peptide | SEQ ID NO: |
|---|---|
| LSDFVPQDIQKL | 829 |
| SDFVPQDIQKL | 830 |
| DFVPQDIQKL | 831 |
| FVPQDIQKLWHS | 832 |
| FVPQDIQKLWH | 833 |
| FVPQDIQKLW | 834 |
| LSDFVPQDIQKLWHS | 835 |
| LSDFVPQDIQKLWH | 836 |
| LSDFVPQDIQKLW | 837 |
| SDFVPQDIQKLWHS | 838 |
| SDFVPQDIQKLWH | 839 |
| SDFVPQDIQKLW | 840 |
| DFVPQDIQKLWHS | 841 |
| DFVPQDIQKLWH | 842 |
| DFVPQDIQKLW | 843 |

SEQ ID NOs:829-843 correspond to SEQ ID NO:53 in which one, two or three additional contiguous amino acids from the Alt a 8 protein sequence are optionally incorporated at the N-terminus, C-terminus and both N- and C-terminus.

| Peptide | SEQ ID NO: |
|---|---|
| KGAYVYFASDAS | 844 |
| GAYVYFASDAS | 845 |
| AYVYFASDAS | 846 |
| YVYFASDASSYV | 847 |
| YVYFASDASSY | 848 |
| YVYFASDASS | 849 |
| KGAYVYFASDASSYV | 850 |
| KGAYVYFASDASSY | 851 |
| KGAYVYFASDASS | 852 |
| GAYVYFASDASSYV | 853 |
| GAYVYFASDASSY | 854 |
| GAYVYFASDASS | 855 |
| AYVYFASDASSYV | 856 |
| AYVYFASDASSY | 857 |
| AYVYFASDASS | 858 |
| YVYFASDASSYC | 859 |
| KGAYVYFASDASSYC | 860 |
| GAYVYFASDASSYC | 861 |
| AYVYFASDASSYC | 862 |

SEQ ID NOs:844-862 correspond to SEQ ID NO:54 in which one, two or three additional contiguous amino acids from the Alt a 8 protein sequence are optionally incorporated at the N-terminus, C-terminus and both N- and C-terminus. A CN substitution has been made in SEQ ID NOs:847, 850, 853, 856.

| Peptide | SEQ ID NO: |
|---|---|
| DKGYFIEPTIFS | 863 |
| KGYFIEPTIFS | 864 |
| GYFIEPTIFS | 865 |
| YFIEPTIFSNVT | 866 |
| YFIEPTIFSNV | 867 |
| YFIEPTIFSN | 868 |
| DKGYFIEPTIFSNVT | 869 |
| DKGYFIEPTIFSNV | 870 |
| DKGYFIEPTIFSN | 871 |
| KGYFIEPTIFSNVT | 872 |
| KGYFIEPTIFSNV | 873 |
| KGYFIEPTIFSN | 874 |
| GYFIEPTIFSNVT | 875 |
| GYFIEPTIFSNV | 876 |
| GYFIEPTIFSN | 877 |

SEQ ID NOs:863-877 correspond to SEQ ID NO:55 in which one, two or three additional contiguous amino acids from the Alt a 10 protein sequence are optionally incorporated at the N-terminus, C-terminus and both N- and C-terminus.

| Peptide | SEQ ID NO: |
|---|---|
| LDNYIQTKTVSI | 878 |
| DNYIQTKTVSI | 879 |
| NYIQTKTVSI | 880 |
| YIQTKTVSIRLG | 881 |
| YIQTKTVSIRL | 882 |
| YIQTKTVSIR | 883 |
| LDNYIQTKTVSIRLG | 884 |
| LDNYIQTKTVSIRL | 885 |
| LDNYIQTKTVSIR | 886 |
| DNYIQTKTVSIRLG | 887 |
| DNYIQTKTVSIRL | 888 |
| DNYIQTKTVSIR | 889 |
| NYIQTKTVSIRLG | 890 |
| NYIQTKTVSIRL | 891 |
| NYIQTKTVSIR | 892 |

SEQ ID NOs:878-892 correspond to SEQ ID NO:56 in which one, two or three additional contiguous amino acids from the Alt a 10 protein sequence are optionally incorporated at the N-terminus, C-terminus and both N- and C-terminus.

| Peptide | SEQ ID NO: |
|---|---|
| QTKTVSIRLGDVLFG | 893 |
| TKTVSIRLGDVLFG | 894 |
| KTVSIRLGDVLFG | 895 |
| TVSIRLGDVLFG | 896 |
| VSIRLGDVLFG | 897 |
| SIRLGDVLFG | 898 |

SEQ ID NOs:893-898 correspond to SEQ ID NO:57 in which one, two, three, four, five or six additional contiguous amino acids from the Alt a 10 protein sequence are optionally incorporated at the N-terminus.

| Peptide | SEQ ID NO: |
|---|---|
| GTVWVNSYNTLH | 899 |
| TVWVNSYNTLH | 900 |
| VWVNSYNTLH | 901 |
| WVNSYNTLHWQL | 902 |
| WVNSYNTLHWQ | 903 |
| WVNSYNTLHW | 904 |
| GTVWVNSYNTLHWQL | 905 |
| GTVWVNSYNTLHWQ | 906 |
| GTVWVNSYNTLHW | 907 |
| TVWVNSYNTLHWQL | 908 |
| TVWVNSYNTLHWQ | 909 |
| TVWVNSYNTLHW | 910 |
| VWVNSYNTLHWQL | 911 |
| VWVNSYNTLHWQ | 912 |
| VWVNSYNTLHW | 913 |

SEQ ID NOs:899-913 correspond to SEQ ID NO:58 in which one, two or three additional contiguous amino acids from the Alt a 10 protein sequence are optionally incorporated at the N-terminus, C-terminus and both N- and C-terminus.

The invention may optionally exclude peptides comprising one or more of the following sequences, or peptides having a contiguous sequence of 7, 8 or 9 amino acids that has one of 90%, 91%, 92%, 93%, 94%, 95%, 96%, 97%, 98%, 99% or 100% sequence identity to one or more of the following sequences:

YYNSLGFNI
(e.g. SEQ ID NOs: 6 and 136-150)
LGFNIKATN
(e.g. SEQ ID NOs: 7 and 151-165)
FNIKATNGG
(e.g. SEQ ID NOs: 8 and 166-180)
IKATNGGTL
(e.g. SEQ ID NOs: 9 and 181-195)
ITYVATATL
(e.g. SEQ ID NOs: 10 and 196-210)
[SEQ ID NO: 914]
VATATLPNY
(e.g. SEQ ID NOs: 199, 202, 205, 208)
YVATATLPN
(e.g. SEQ ID NOs: 11 and 211-233)
YITLVTLPK
(e.g. SEQ ID NOs: 16 and 294-307)
ITLVTLPKS
(e.g. SEQ ID NOs: 17 and 308-317)
[SEQ ID NO: 915]
VYQKLKALA
(e.g. SEQ ID NOs: 724-726, 730-738)
YQKLKALAK
(e.g. SEQ ID NOs: 46 and 724-738)
[SEQ ID NO: 916]
KLKALAKKT
(e.g. SEQ ID NOs: 727, 728, 730, 731, 733, 732,
736, 737
[SEQ ID NO: 917]
LKALAKKTY
(e.g. SEQ ID NOs: 727, 730, 733, 736
[SEQ ID NO: 918]
FGAGWGVMV
WGVMVSHRS
(e.g. SEQ ID NOs: 49 and 769-783)
WGVLVSHRS
(e.g. SEQ ID NOs: 50 and 784-798)
[SEQ ID NO: 919]
GVMVSHRSG
(e.g. SEQ ID NOs:
[SEQ ID NO: 920]
VMVSHRSGE
(e.g. SEQ ID NOs: 772, 773, 775, 776, 778, 779,
781, 782)
[SEQ ID NO: 921]
MVSHRSGET
(e.g. SEQ ID NOs: 772, 775, 778, 781)
[SEQ ID NO: 922]
YVWKISEFY
[SEQ ID NO: 923]
LLLKQKVSD
[SEQ ID NO: 924]
LLKQKVSDD
[SEQ ID NO: 925]
VVLVAYFAA
[SEQ ID NO: 926]
VVGRQILKS
[SEQ ID NO: 927]
VVGRQIMKS

The invention may optionally exclude peptides comprising one or more of SEQ ID NOs:933-1163 (FIG. 27), or peptides having a contiguous sequence of 7, 8 or 9 amino acids that has one of 90%, 91%, 92%, 93%, 94%, 95%, 96%, 97%, 98%, 99% or 100% sequence identity to one or more of SEQ ID Nos:933-1163 (FIG. 27).

In another aspect of the present invention a peptide is provided wherein the peptide comprises the sequence of one of SEQ ID NOs:1-913. The amino acid sequence of the selected SEQ ID NO is preferably included in the peptide as a contiguous amino acid sequence.

In another aspect of the present invention a peptide is provided having at least 60% amino acid sequence identity to one of SEQ ID NOs:1-913. More preferably, the degree of sequence identity is one of 65%, 70%, 75%, 80%, 85%, 87%, 90%, 91%, 92%, 93%, 94%, 95%, 96%, 97%, 98%, 99% or 100% identity.

A peptide according to the present invention may have a maximum length of 50 amino acids and less than the full length of the corresponding protein allergen. More preferably the maximum peptide length is 40 amino acids, still more preferably 30 amino acids, still more preferably the maximum length is chosen from one of 29, 28, 27, 26, 25, 24, 23, 22, 21, 20, 19, 18, 17, 16, 15, 14, 13, 12, 11, 10 or 9 amino acids. For example, a peptide may have a maximum length of 20 amino acids or 15 amino acids.

A peptide according to the present invention may have a minimum length of 7 amino acids. More preferably the minimum length is chosen from one of 7, 8, 9, 10, 11, 12, 13, 14, 15, 16, 17, 18, 19 or 20 amino acids.

A peptide according to the present invention may have any length between said minimum and maximum. Thus, for example, a peptide may have a length of from 8 to 30, 10 to 25, 12 to 20, 9 to 15 amino acids, 8 to 11 amino acids, 9 to 11 amino acids, 9 to 13 amino acids or 9 to 14 amino acids. In particular, the peptide may have an amino acid length chosen from one of 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids, such as 9 or 15 amino acids.

The present invention incorporates peptide derivatives and peptide mimetics of any one of SEQ ID NOs:1-913.

Peptide derivatives include variants of a given SEQ ID NO and may include naturally occurring allelic variants and synthetic variants which have substantial amino acid sequence identity to the peptide sequence as identified in the wild type full length protein allergen.

Peptide derivatives may include those peptides having at least 60% amino acid sequence identity to one of SEQ ID NOs:1-913 and which are capable of stimulating an immune response.

Typically a peptide derivative shows similar or improved MHC binding compared to the parent sequence, e.g. one of SEQ ID NOS:1-913. Preferably a peptide derivative shows promiscuous binding to MHC Class II molecules.

Peptide derivatives may include peptides having at least one amino acid modification (e.g. addition, substitution, and/or deletion of one or more amino acids) compared to one of SEQ ID NOs:1-913.

Peptide derivatives preferably differ from one of SEQ ID NOS:1-913 by less than 5 amino acids. More preferably, the number of different amino acids is 4 amino acids or less, 3 amino acids or less, 2 amino acids or less or only 1 amino acid.

Peptide derivatives may arise through natural variations or polymorphisms which may exist between the members of a protein allergen family from which the peptide is derived. All such derivatives are included within the scope of the invention.

Peptide derivatives may result from natural or non-natural (e.g. synthetic) interventions leading to addition, replacement, deletion or modification of the amino acid sequence of one of SEQ ID NOs:1-913.

Conservative replacements and modifications which may be found in such polymorphisms may be between amino acids within the following groups:
(i) alanine, serine, threonine;
(ii) glutamic acid and aspartic acid;
(iii) arginine and leucine;
(iv) asparagine and glutamine;
(v) isoleucine, leucine and valine;
(vi) phenylalanine, tyrosine and tryptophan;
(vii) methionine and leucine;
(viii) cysteine and valine.

Peptide derivatives may be peptide truncates of one or more of SEQ ID NOs:1-913, e.g. one or more of SEQ ID NOs:59-913. A peptide truncate has the same amino acid sequence as one of SEQ ID NOs:1-913 except for the deletion of one or more amino acids. 1, 2, 3, 4, or 5 amino acids may be deleted to provide a peptide truncate. A set of peptide truncates may be prepared in which 1, 2, 3, 4 or 5 amino acids are absent from either the C- or N-terminus of one of SEQ ID Nos:1-913, e.g. one of SEQ ID NOs:59-913 to provide a set of up to 10 peptide truncates. Whilst peptide truncates may be prepared by removing the required number of amino acids from the C- or N-terminus of one of SEQ ID NOs:1-913 it is preferred to directly synthesise the required shorter peptide in accordance with the amino acid sequence of the desired peptide truncate.

Peptide truncates can also be synthesised to have a sequence that corresponds to one of SEQ ID NOs:1-913, e.g. one of SEQ ID NOs:59-913, where 1, 2, 3, 4 or 5 amino acids in internal positions in the peptide are deleted.

Peptide derivatives may also be provided by modifying one of SEQ ID NOs:1-913 to resist degradation of the peptide. FIG. 24 summarises modifications that may be made to the peptides to help resist peptide degradation and enhance peptide half-life in vitro and in vivo. These modifications may improve in vitro peptide stability and long-term storage. FIG. 24 also indicates enhancing sequences that may increase the rate of reaction of an adjacent or nearby amino acid.

Peptide derivatives may be provided by modifying one of SEQ ID NOs:1-913 for protease resistance, for example by inclusion of chemical blocks for exoproteases.

SEQ ID NOs:444, 609, 688, 718, 790, 820 are derivatives in that each peptide comprises an M/L substitution compared to the corresponding parent allergen sequence.

Similarly, SEQ ID NOs:217, 255, 285, 384, 414, 444, 639, 760, 850 are derivatives in that each peptide comprises a ON substitution compared to the parent allergen sequence.

Peptide derivatives may also be provided by modifying one of SEQ ID NOs:1-913, e.g. one of SEQ ID NOs:59-913, to alter the immunomodulatory properties of the peptide. These derivatives are sometimes referred to as altered peptide ligands (APLs) (25). APLs typically produce an altered immune response compared to the unaltered (e.g. wild type) peptide. For example, an APL may induce increased or decreased T cell activation, altered cytokine profile in activated T cells, and/or altered MHC binding compared to the unaltered peptide. Preferably an APL displays promiscuous binding of MHC molecules as described herein.

Peptide derivatives may be assayed for their ability to induce an immune response, e.g. T cell proliferation and/or cytokine production in a T cell population, in order to identify a peptide pharmacophore representing the minimal or optimised peptide epitope capable of stimulating an immune response and that may be useful in therapy. The immune response induced by a peptide may be one or more of:
(i) in vitro T cell proliferation, e.g. as measured by peptide stimulation of bromodeoxyuridine or ³H-thymidine incorporation in in vitro cultured PBMC, and/or
(ii) secretion of cytokines, e.g. IFNγ and/or IL-4, by in vitro cultured PBMC or T cells, e.g. T helper cells, and/or
(iii) a Th1 or Th2 response (e.g. as measured by secretion of cytokines such as IFNγ or IL-4 respectively).

Peptide derivatives such as APLs may be screened for MHC binding, in particular for binding to HLA Class II molecules.

The invention includes a method of identifying a peptide derivative of one of SEQ ID NOs:1-913 that is capable of stimulating an immune response. The method comprises the steps of (i) providing a candidate peptide derivative and (ii) testing the ability of the candidate peptide derivative to induce an immune response.

Part (i) may comprise synthesising the candidate peptide derivative, which may be a peptide mimetic or APL. Alternatively, part (i) may comprise chemically modifying the structure of one of SEQ ID NOs:1-913 so as to produce a candidate peptide derivative. Part (i) may comprise synthesis of peptide truncates or derivatives. The candidate peptide derivative will preferably have at least 60% sequence identity to one of SEQ ID NOs:1-913.

Chemical modification of one of SEQ ID NOs:1-913 may, for example, comprise deletion of one or more amino acids, addition of one or more amino acids or chemical modification of one or more amino acid side chains.

Part (ii) may comprise screening a candidate peptide derivative for MHC binding, in particular for binding to HLA Class II molecules. Especially, part (ii) may comprise testing a candidate peptide derivative for promiscuous binding to MHC Class II molecules. In silico screening may be carried out using virtual HLA Class II matrices, such as the ProPred software described herein. An in vitro binding assay may be used to assess binding to HLA Class II molecules, such as the ProImmune Reveal® assay described herein.

Preferably a peptide derivative, e.g. an APL, is a promiscuous binder of MHC Class II alleles. Typically a promiscuous binding epitope binds over 50%, for example, at least 60% or at least 70%, of the HLA-DR alleles expressed by European Americans. The 11 most common alleles expressed by European Americans are shown in FIG. 25. Preferably a promiscuous binding epitope binds one of at least 2, 3, 4, 5, 6, 7, 8, 9, 10 or all 11 of the HLA-DR alleles in FIG. 25. In one aspect a peptide derivative may bind at least 2, 3, 4, 5, 6, 7, 8, 9, 10 or 11 of the alleles in FIG. 25 and also the HLA-DR allele *1401. The method may therefore comprise selecting a peptide that binds promiscuously.

Part (ii) may comprise contacting the candidate peptide derivative with a population of T cells and assaying T cell proliferation. Additionally, or alternatively, part (ii) may comprise contacting the candidate peptide derivative with a population of T cells and monitoring cytokine production, such as production of IFNγ and/or IL-4. The T cells are preferably T helper cells. The T cells may be provided as an in vitro culture of PBMC.

The method may further comprise the step of selecting one or more candidate peptide derivatives that stimulate T cell proliferation and detecting the production of cytokines in order to determine the induction of a Th1 or Th2 response. Preferably, the method comprises detection of IFNγ and/or IL-4. The method may further comprise selecting a peptide that induces a Th1 or Th2 response.

Methods according to the present invention may be performed in vitro or in vivo. The term “in vitro” is intended to encompass experiments with cells in culture whereas the term “in vivo” is intended to encompass experiments with intact multi-cellular organisms. Where the method is performed in vitro it may comprise a high throughput screening assay. Test compounds used in the method may be obtained from a synthetic combinatorial peptide library, or may be synthetic peptides or peptide mimetic molecules. Method steps (i) and (ii) are preferably performed in vitro, e.g. in cultured cells. Cells may be of any suitable cell type, e.g. mammalian, bacterial or fungal. Host cell(s) may be non-human, e.g. rabbit, guinea pig, rat, mouse or other rodent (including cells from any animal in the order Rodentia), cat, dog, pig, sheep, goat, cattle, horse, non-human primate or other non-human vertebrate organism; and/or non-human mammalian; and/or human. Suitable cells, e.g. PBMCs, may be obtained by taking a blood sample.

Part (ii) of the method may additionally comprise testing a candidate peptide derivative in animal models or patient populations for therapeutic effects on fungal allergy or fungal infection.

Peptides according to the present invention may be useful in the prevention or treatment of disease. In particular, peptides according to the present invention may be used to prepare pharmaceutical compositions. The pharmaceutical compositions may comprise medicaments or vaccines.

A pharmaceutical composition may be provided comprising a predetermined quantity of one or more peptides according to the present invention. Pharmaceutical compositions according to the present invention may be formulated for clinical use and may comprise a pharmaceutically acceptable carrier, diluent or adjuvant.

Pharmaceutical compositions of the invention are purified reproducible preparations which are suitable for human therapy. Preferred compositions of the invention comprise at least one isolated, purified peptide, free from all other polypeptides or contaminants, the peptide having a defined sequence of amino acid residues which comprises at least one T cell epitope of an antigen of interest.

As used herein, the term “isolated” refers to a peptide which is free of all other polypeptides, contaminants, starting reagents or other materials, and which is not conjugated to any other molecule.

A pharmaceutical composition of the invention is capable of down regulating an antigen specific immune response to an antigen of interest in a population of humans or animals subject to the antigen specific immune response such that disease symptoms are reduced or eliminated, and/or the onset or progression of disease symptoms is prevented or slowed.

Compositions and methods of the invention may be used to treat sensitivity to protein allergens in humans such as allergies to fungi, particularly to Alternaria spp.

Accordingly, in a further aspect of the invention a peptide according to the present invention is provided for use in the prevention or treatment of disease.

In another aspect of the present invention a peptide according to the present invention is provided for use in a method of medical treatment. The medical treatment may comprise treatment of a disease, e.g. allergic disease.

In another aspect of the present invention the use of a peptide according to the present invention in the manufacture of a medicament for the prevention or treatment of disease is provided.

In another aspect of the present invention a method is provided for preventing or treating disease in a patient in need of treatment, the method comprising administering to the patient a therapeutically effective amount of a peptide or pharmaceutical composition according to the present invention.

In accordance with the present invention methods are also provided for the production of pharmaceutically useful compositions, which may be based on a peptide or peptide derivative according to the present invention. In addition to the steps of the methods described herein, such methods of production may further comprise one or more steps selected from:
(a) identifying and/or characterising the structure of a selected peptide or peptide derivative;
(b) obtaining the peptide or peptide derivative;
(c) mixing the selected peptide or peptide derivative with a pharmaceutically acceptable carrier, adjuvant or diluent.

For example, a further aspect of the present invention relates to a method of formulating or producing a pharmaceutical composition for use in the treatment of disease, the method comprising identifying a peptide or peptide derivative in accordance with one or more of the methods described herein, and further comprising one or more of the steps of:
(i) identifying the peptide or peptide derivative; and/or
(ii) formulating a pharmaceutical composition by mixing the selected peptide or peptide derivative, with a pharmaceutically acceptable carrier, adjuvant or diluent.

As such, the method may comprise providing a peptide which peptide comprises the sequence of one of SEQ ID NOs:1-913, and formulating a pharmaceutical composition by mixing the selected peptide or peptide derivative with a pharmaceutically acceptable carrier, adjuvant or diluent.

The peptide or peptide derivative may be present in the pharmaceutical composition in the form of a physiologically acceptable salt.

Methods of medical treatment may involve administering more than one peptide according to the invention to the patient. Administering two, three or more peptides derived from a single allergen, or derived from multiple allergens, may be used to ensure that peptide epitopes that bind to a large number of HLA alleles are provided. For example, one may wish to ensure that the treatment includes administration of peptide epitopes derived from a given allergen that collectively bind to all 11 alleles of FIG. 25. Administration of multiple peptides may be simultaneous, separate or sequential and may form part of a combination therapy.

Accordingly, a pharmaceutical composition or medicament according to the invention may comprise more than one peptide epitope of the invention and may be active against one or more allergens. The one or more allergens may be derived from one or more species, e.g. fungal species such as Alternaria spp. Such compositions and medicaments may contain more than one peptide and/or peptide derivative and/or peptide mimetic according to the invention, for example, 1, 2, 3, 4, 5, 6, 7, 8, 9, 10, 11, 12, 13, 14, 15, 16, 17, 18, 19, 20 or more peptides, peptide derivatives and/or peptide mimetics. These may be derived from the same or different protein allergen(s).

For example, in some embodiments a pharmaceutical composition may comprise 2, 3, 4, 5, 6, 7, 8, 9, 10, 11, 12, 13, 14, 15, 16, 17, 18, 19, 20, 21, 22, 23, 24, 25, 26, or 27 peptides selected from the following list but wherein no more than three (preferably no more than two or one) peptide(s) from each of the following numbered groups is present:
(i) SEQ ID NO: 1, SEQ ID NOs: 59-80
(ii) SEQ ID NO: 2, SEQ ID NOs: 81-92
(iii) SEQ ID NO: 3, SEQ ID NOs: 93-105
(iv) SEQ ID NO: 4, SEQ ID NOs: 106-120
(v) SEQ ID NO: 5, SEQ ID NOs: 121-135
(vi) SEQ ID NO: 6, SEQ ID NOs: 136-150
(vii) SEQ ID NO: 7, SEQ ID NOs: 151-165
(viii) SEQ ID NO: 8, SEQ ID NOs: 166-180
(ix) SEQ ID NO: 9, SEQ ID NOs: 181-195
(x) SEQ ID NO: 10, SEQ ID NOs: 196-210
(xi) SEQ ID NO: 11, SEQ ID NOs: 211-233
(xii) SEQ ID NO: 12, SEQ ID NOs: 234-248
(xiii) SEQ ID NO: 13, SEQ ID NOs: 249-263
(xiv) SEQ ID NO: 14, SEQ ID NOs: 264-278
(xv) SEQ ID NO: 15, SEQ ID NOs: 279-293
(xvi) SEQ ID NO: 16, SEQ ID NOs: 294-307
(xvii) SEQ ID NO: 17, SEQ ID NOs: 308-317
(xviii) SEQ ID NO: 18, SEQ ID NOs: 318-332
(xix) SEQ ID NO: 19, SEQ ID NOs: 333-347
(xx) SEQ ID NO: 20, SEQ ID NOs: 348-362
(xxi) SEQ ID NO: 21, SEQ ID NOs: 363-377
(xxii) SEQ ID NO: 22, SEQ ID NOs: 378-392
(xxiii) SEQ ID NO: 23, SEQ ID NOs: 393-407
(xxiv) SEQ ID NO: 24, SEQ ID NOs: 408-422
(xxv) SEQ ID NO: 25, SEQ ID NOs: 423-437
(xxvi) SEQ ID NO: 26, SEQ ID NOs: 438-452
(xxvii) SEQ ID NO: 27, SEQ ID NOs: 453-467.

In some embodiments a pharmaceutical composition may comprise 2, 3, 4, 5, 6, 7, 8, 9, or 10 peptides selected from the following list but wherein no more than three (preferably no more than two or one) peptides from each of the following numbered groups is present:
(i) SEQ ID NO: 4, SEQ ID NOs: 106-120
(ii) SEQ ID NO: 5, SEQ ID NOs: 121-135
(iii) SEQ ID NO: 8, SEQ ID NOs: 166-180
(iv) SEQ ID NO: 9, SEQ ID NOs: 181-195
(v) SEQ ID NO: 11, SEQ ID NOs: 211-233
(vi) SEQ ID NO: 13, SEQ ID NOs: 249-263
(vii) SEQ ID NO: 22, SEQ ID NOs: 378-392
(viii) SEQ ID NO: 24, SEQ ID NOs: 408-422
(ix) SEQ ID NO: 26, SEQ ID NOs: 438-452
(x) SEQ ID NO: 27, SEQ ID NOs: 453-467.

In some embodiments a pharmaceutical composition may comprise 2, 3, 4, 5, 6, 7, 8 or 9 peptides selected from the following list but wherein no more than three (preferably no more than two or one) peptides from each of the following numbered groups is present:
(i) SEQ ID NO: 1, SEQ ID NOs: 59-80
(ii) SEQ ID NO: 6, SEQ ID NOs: 136-150
(iii) SEQ ID NO: 7, SEQ ID NOs: 151-165
(iv) SEQ ID NO: 10, SEQ ID NOs: 196-210
(v) SEQ ID NO: 15, SEQ ID NOs: 279-293
(vi) SEQ ID NO: 16, SEQ ID NOs: 294-307
(vii) SEQ ID NO: 18, SEQ ID NOs: 318-332
(viii) SEQ ID NO: 19, SEQ ID NOs: 333-347
(ix) SEQ ID NO: 20, SEQ ID NOs: 348-362.

In some embodiments a pharmaceutical composition may comprise 2, 3, 4, 5, 6, 7, 8, 9, 10, 11, or 12 peptides selected from the following list but wherein no more than three (preferably no more than two or one) peptides from each of the following numbered groups is present:
(i) SEQ ID NO: 28, SEQ ID NOs: 468-482
(ii) SEQ ID NO: 31, SEQ ID NOs: 513-527
(iii) SEQ ID NO: 32, SEQ ID NOs: 528-542
(iv) SEQ ID NO: 33, SEQ ID NOs: 543-557
(v) SEQ ID NO: 35, SEQ ID NOs: 573-587
(vi) SEQ ID NO: 37, SEQ ID NOs: 603-617
(vii) SEQ ID NO: 43, SEQ ID NOs: 697-711
(viii) SEQ ID NO: 45, SEQ ID NOs: 718-723
(ix) SEQ ID NO: 46, SEQ ID NOs: 724-738
(x) SEQ ID NO: 50, SEQ ID NOs: 784-798
(xi) SEQ ID NO: 53, SEQ ID NOs: 829-843
(xii) SEQ ID NO: 57, SEQ ID NOs: 893-898.

In some embodiments a pharmaceutical composition may comprise 2, 3, 4, 5, 6, 7, 8, 9, 10, 11, or 12 peptides selected from the following list but wherein no more than three (preferably no more than two or one) peptides from each of the following numbered groups is present:
(i) SEQ ID NO: 29, SEQ ID NOs: 483-497
(ii) SEQ ID NO: 30, SEQ ID NOs: 498-512
(iii) SEQ ID NO: 34, SEQ ID NOs: 558-572
(iv) SEQ ID NO: 39, SEQ ID NOs: 633-647
(v) SEQ ID NO: 40, SEQ ID NOs: 648-662
(vi) SEQ ID NO: 42, SEQ ID NOs: 682-696
(vii) SEQ ID NO: 48, SEQ ID NOs: 754-768
(viii) SEQ ID NO: 52, SEQ ID NOs: 814-828
(ix) SEQ ID NO: 54, SEQ ID NOs: 844-862
(x) SEQ ID NO: 55, SEQ ID NOs: 863-877
(xi) SEQ ID NO: 56, SEQ ID NOs: 878-892
(xii) SEQ ID NO: 58, SEQ ID NOs: 899-913.

In yet a further aspect of the present invention nucleic acids encoding peptides according to the present invention are provided, together with their complementary sequences. The nucleic acid may have a maximum length of 1000 nucleotides, more preferably one of 200, 190, 180, 170, 160, 150, 140, 130, 120, 110, 100, 90, 80, 70, 60, 50, 40, 30, 25 nucleotides. The nucleic acid may have a minimum length of 24 nucleotides, more preferably one of 27, 30, 35, 40, 45, 50, 55 or 60 nucleotides.

A nucleic acid vector having nucleic acid encoding a peptide of the present invention is also provided. The vector may be an expression vector, e.g. a plasmid, in which a nucleic acid sequence encoding a peptide of the present invention is operably linked to a suitable promoter and/or other regulatory sequence. A host cell transfected with such a vector is also provided.

In this specification the term “operably linked” may include the situation where a selected nucleotide sequence and regulatory or control nucleotide sequence are covalently linked in such a way as to place the expression of a nucleotide sequence under the influence or control of the regulatory sequence. Thus a regulatory or control sequence is operably linked to a selected nucleotide sequence if the regulatory sequence is capable of effecting transcription of a nucleotide sequence which forms part or all of the selected nucleotide sequence. Where appropriate, the resulting transcript may then be translated into a desired peptide.

The vector may be configured to enable transcription of mRNA encoding the peptide upon transfection into a suitable cell. Transcribed mRNA may then be translated by the cell such that the cell expresses the peptide.

A cell having a nucleic acid sequence encoding a peptide of the present invention operably linked to a suitable promoter and/or other transcription regulatory element or control sequence integrated in the genome of the cell is also provided.

Nucleic acids according to the invention may be single or double stranded and may be DNA or RNA.

Diseases or conditions that may be prevented or treated include allergic disease. Examples of allergic disease include asthma, allergic asthma, fungal asthma, SAFS, ABPA, allergic bronchopulmonary mycoses, allergic sinusitis, rhinitis, allergic rhinitis, hypersensitivity pneumonitis, atopic eczema. Other diseases or conditions that may be prevented or treated include fungal infection, Aspergillosis (e.g. invasive, non-invasive, chronic pulmonary, aspergilloma).

Peptide therapy may comprise the use of peptides according to the invention in the prevention/prophylaxis of disease or in the treatment of disease. As such, therapy may comprise relief or reduction of symptoms such as airway inflammation, difficulty in breathing, swelling, itchiness, allergic rhinitis, allergic sinusitis, eosinophilia, hypersensitivity to fungal allergens and/or spores. A reduction in asthmatic symptoms may be measured by conventional techniques, such as measuring peak flow, white blood cell count, patch testing.

Peptides according to the present invention may be useful as prophylactics for the prevention of allergy responses to fungal allergens, particularly allergens from Alternaria alternata.

Patients to be treated may be any animal or human. The patient may be a non-human mammal, but is more preferably a human. Subjects, individuals or patients to be treated may be male or female. In one aspect, patients are of a selected ethnicity, which may include one or more of (by birth or residence): (i) European, (ii) from a Member State of the European Union, (iii) North American, e.g. from USA and/or Canada. Patients to be treated may be European American and/or Caucasian.

Medicaments and pharmaceutical compositions according to aspects of the present invention may be formulated for administration by a number of routes, including intravenous, intradermal, intramuscular, oral and nasal. The medicaments and compositions may be formulated in fluid or solid form. Fluid formulations may be formulated for administration by injection to a selected region of the human or animal body. Pharmaceutical compositions may comprise peptides encapsulated in liposomes, e.g. formed from polyglycerol esters.

Administration of peptides or pharmaceutical compositions for therapeutic purposes is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

A composition may be administered alone or in combination with other treatments, either simultaneously or sequentially, dependent upon the condition to be treated.

Efficacious peptide immunotherapy may require the repeat administration of a pharmaceutical composition according to the present invention. For example, a dosage regime comprising a series of injections of the pharmaceutical composition may be required in order to treat existing allergic disease symptoms and to provide a vaccination effect against future allergic disease caused by fungal allergens.

Peptides comprising or consisting of SEQ ID NOS:1-913 are disclosed along with variants and derivatives thereof, including peptides having conservative alterations. These peptides are each proposed for use in the treatment of fungal allergy, preferably allergic disease caused by A. alternata.

The peptides identified may be synthesised by standard techniques (e.g. using commercially available peptide synthesis services such as that provided by Invitrogen, Carlsbad, Calif., USA) and tested for use as a therapeutic or vaccine against fungal infection or fungal allergy.

Various methods of chemically synthesizing peptides are known in the art such as solid phase synthesis which has been fully or semi-automated on commercially available peptide synthesizers. Synthetically produced peptides may then be purified to homogeneity (i.e. at least 90%, more preferably at least 95% and even more preferably at least 97% purity), free from all other polypeptides and contaminants.

Peptide compositions may then be characterized by a variety of techniques well known to those of ordinary skill in the art such as mass spectroscopy, amino acid analysis and sequencing and HPLC.

Peptides useful in the methods of the present invention may also be produced using recombinant DNA techniques in a host cell transformed with a nucleic acid sequence coding for such peptide. When produced by recombinant techniques, host cells transformed with nucleic acid encoding the desired peptide are cultured in a medium suitable for the cells and isolated peptides can be purified from cell culture medium, host cells, or both using techniques known in the art for purifying peptides and proteins including ion-exchange chromatography, ultra filtration, electrophoresis or immunopurification with antibodies specific for the desired peptide. Peptides produced recombinantly may be isolated and purified to homogeneity, free of cellular material, other polypeptides or culture medium for use in accordance with the methods described above.

Pharmaceutical compositions of the invention should be sterile, stable under conditions of manufacture, storage, distribution and use and should be preserved against the contaminating action of microorganisms such as bacteria and fungi. A preferred means for manufacturing a pharmaceutical composition of the invention in order to maintain the integrity of the composition is to prepare the formulation of peptide and pharmaceutically acceptable carrier(s) such that the composition may be in the form of a lyophilized powder which is reconstituted in a pharmaceutically acceptable carrier, such as sterile water, just prior to use.

Biodegradable poly(D,L-lactic-co-glycolic) acid (PGLA) particles has been suggested for delivery of peptides for treatment of allergy (Scholl et al. Immunol. Allergy Clin. N. Am. 2006. 26:349-364).

T-cell epitope validation can be performed by assaying peptide-induced proliferation of peripheral blood mononuclear cells (PBMC) obtained from subjects having fungal allergy or fungal infection and from control subjects not having fungal allergy or fungal infection. HLA-DR typing of subject PBMCs may also be performed to confirm the promiscuous binding nature of the peptides.

The status of the proliferated T helper cells may also be determined and used to assist in validation of peptides as therapeutic or vaccine candidates. Th1 cells participate in cell-mediated immunological responses. Th2 cells participate in antibody mediated immunity.

Th1/Th2 status can be determined by examining the cytokine profile of the proliferated cells (27). Production of interferon γ (IFNγ) and optionally one or more of interleukin 2 (IL-2), tumor necrosis factor β (TNFβ) and granulocyte-macrophage colony stimulating factor (GM-CSF) is indicative of Th1 status. Typically this indicates a non-allergic cellular immune response. Production of interleukin 4 (IL-4) and optionally one or more of interleukin 3 (IL-3), interleukin 5 (IL-5), interleukin 6 (IL-6), interleukin 10 (IL-10) and interleukin 13 (IL-13) is indicative of Th2 status. Often this is associated with an allergic Th2 response. Production of both IFNγ and IL-4 is indicative of Th0 status. Production of IL-10 is associated with a Treg non-allergic response. (27)

Th2 cells play an important role in the immunological processes of allergic asthma (11) and Th2 associated cytokines such as IL-4, IL-5, IL-9 and IL-13 are implicated in the development of allergen specific Th2 cells, IgE production, airway eosinophilia and airway hyper-responsiveness. Inhibition or suppression of allergen-specific Th2 cells and their cytokines provides a strategy for intervention.

Such inhibition or suppression may be achieved by selecting Th1 stimulating peptides leading to suppression of the Th2 response (11). Alternatively, Th2 stimulating peptides administered via different routes (oral, lymph node injection or intravenous) and by specific dose variation may be used to suppress an allergen induced Th2 response through a bystander effect. The bystander effect is defined as an influence on the immune response to a particular antigen(s) of interest by the immune response to other unrelated antigens, usually mediated by a local cytokine and cellular environment. The bystander effect can result in an amplification of an immune response (22) or a suppression of a response (23).

Low-dose T-cell epitope peptides from allergen proteins are proposed to cause antigen specific hypo-responsiveness associated with the induction of a suppressive population of CD4+ T cells, together with up regulation of surface CD5 levels on antigen-specific T cells (12). Intravenous injection of a single peptide induces a bystander suppression and thus can provide protection against a multicomponent allergen trigger (13).

Accordingly, in addition to assaying for T cell proliferation (e.g. based on Bromodeoxyuridine (BRdU) or ³H thymidine incorporation), cytokine assays' may be performed to detect secretion of one or more of IFNγ, IL-2, TNFβ, GM-CSF, IL-4, IL-3, IL-5, IL-6, IL-10 and IL-13. Further assays to detect the presence of an IgE response and/or eosinophilia may also be performed.

Human T cell stimulating activity can be tested by culturing T cells obtained from an individual sensitive to a predetermined protein antigen with a peptide derived from the antigen and determining whether proliferation of T cells occurs in response to the peptide as measured, e.g., by cellular uptake of ³H thymidine. Stimulation indices for responses by T cells to peptides can be calculated as the maximum counts per minute (CPM) in response to a peptide divided by the control CPM. A T cell stimulation index (S.I.) equal to or greater than two times the background level is considered “positive”. Positive results are used to calculate the mean stimulation index for each peptide for the group of peptides tested.

Preferred peptides have a mean T cell stimulation index of greater than or equal to 2.0. A peptide having a T cell stimulation index of greater than or equal to 2.0 is considered useful as a therapeutic agent. Preferred peptides have a mean T cell stimulation index of at least 2.5, more preferably at least 3.5, even more preferably at least 4.0, and most preferably at least 5.0.

The positivity index (P.I.) for a peptide is determined by multiplying the mean T cell stimulation index by the percent of individuals, in a population of individuals tested, sensitive to the antigen being tested (e.g., preferably at least 9 individuals, more preferably at least 16 individuals or more, more preferably at least 20 individuals or more, or even more preferably at least 30 individuals or more), who have T cells that respond to the peptide. The positivity index represents the strength of a T cell response to a peptide (S.I.) and the frequency of a T cell response to a peptide in a population of individuals sensitive to the antigen being tested. Preferred peptides may also have a positivity index (P.I.) of at least about 100, more preferably at least 150, even more preferably at least about 200 and most preferably at least about 250.

Cytokine production may be analysed using any of the methods described herein. One such method employs an Enzyme-linked ImmunoSpot (ELISPOT) assay. The ELISPOT assay will allow the analysis of cells at the single cell level for cytokine production, and thus provides a method for determining the number of individual T cells secreting a cytokine after stimulation with a specific antigen or peptide (28). The ELISPOT assay typically uses two high-affinity cytokine-specific antibodies directed against different epitopes on the same cytokine molecule. Spots are generated with a colorimetric reaction in which soluble substrate is cleaved, leaving an insoluble precipitate at the site of the reaction. The spot represents a foot-print of the original cytokine producing cell. The number of spots is a direct measurement of the frequency of cytokine-producing T cells.

The production of cytokines by T-cells in PMBC cell cultures in response to allergen indicates that stimulation has occurred and identification of the cytokine pattern allows a comparison of the type of cellular response.

Peptides selected through in vitro validation assays such as those described above may be tested in animal models or patient populations for therapeutic effects on fungal allergy or fungal infection, e.g. as described in Kheradmand et al (24). For example, a mouse model may be used, such as BALB/c(H2^{d}) mice. Patients or animals may receive a series of peptide formulations, e.g. by injection, and fungal infection or allergy symptoms and characteristics monitored. Such symptoms and characteristics may include airway inflammation, eosinophilia, rhinitis, cytokine secretion, Th1 or Th2 response status. Suitably, a control patient population receiving placebo formulations may be used to assess efficacy of the peptide formulation.

### Peptide Mimetics

The designing of mimetics to a known pharmaceutically active compound is a known approach to the development of pharmaceuticals based on a “lead” compound. This might be desirable where the active compound is difficult or expensive to synthesise or where it is unsuitable for a particular method of administration, e.g. some peptides may be unsuitable active agents for oral compositions as they tend to be quickly degraded by proteases in the alimentary canal. Mimetic design, synthesis and testing is generally used to avoid randomly screening large numbers of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled according to its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this in the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound. The mimetic or mimetics found by this approach can then be screened to see whether they have the target property, or to what extent they exhibit it. Further optimisation or modification can then be carried out to arrive at one or more final mimetics for in vivo or clinical testing.

With regard to the present invention, a peptide mimetic is one form of peptide derivative. A method of identifying a peptide derivative capable of stimulating an immune response may comprise the step of modifying the peptide structure to produce a peptide mimetic. This peptide mimetic may optionally be subject to testing in a T cell proliferation assay, and/or in cytokine secretion assays (e.g. assaying for IFNγ or IL-4 production). This process of modification of the peptide or peptide mimetic and testing may be repeated a number of times, as desired, until a peptide having the desired effect, or level of effect, on T cell proliferation and/or cytokine secretion is identified.

The modification steps employed may comprise truncating the peptide or peptide mimetic length (this may involve synthesising a peptide or peptide mimetic of shorter length), substitution of one or more amino acid residues or chemical groups, and/or chemically modifying the peptide or peptide mimetic to increase stability, resistance to degradation, transport across cell membranes and/or resistance to clearance from the body.

### Altered Peptide Ligands (APLs)

Altered peptide ligands (APLs) are modified versions of peptide epitopes, with altered immunomodulatory properties (25).

A Th1-skewing APL has been reported, having a single 336N/A substitution compared to the wild type peptide epitope (implicated in allergic asthma) and which inhibits the allergic Th2 response in a mouse model of allergic asthma (11).

An APL of an immunodominant epitope of lipocalin allergen Bos d2 has also been reported which produces a Th1/Th0 response in vitro compared to the Th2/Th0 response induced by the wild type epitope (29). The T cell population induced by the APL are cross-reactive with the wild type epitope (29).

Changes in the residues flanking the core epitope of the immunodominant myelin basic protein (MBP) peptide 84-102 have been reported to alter both MHC binding and T cell activation, the latter independently of MHC binding (30). It is suggested that C-terminal basic residues may enhance processing and presentation of an epitope.

With regard to the present invention, an APL is one form of peptide derivative.

An APL typically induces an altered immune response compared to the unaltered (usually wild type) peptide.

Immunomodulatory properties that may be altered include one or more of:

T cell Activation

T cell activation in response to the APL may be increased or decreased compared to the unmodified peptide. Activation may occur at a higher or lower dose of peptide. Some APLs are unable to originate T cell signalling and lead to an impairment of T cell activation (antagonist APLs). Some APLs elicit some but not all of the signals for full T cell activation (partial agonist APLs) (25).

### Cytokine Profile

T cells activated by the peptide may secrete a different pattern of cytokines than T cells activated by the unmodified peptide. Thus, a modified peptide may induce a different type of T cell response, e.g. Th1 in place of Th2, Treg in place of Th2, or Th1 in place of Treg.

### MHC Binding

An APL may show altered MHC binding compared to the unmodified peptide. In the present case it is preferred that an APL shows similar or improved MHC binding compared to the unaltered peptide. In particular it is preferred that an APL is a promiscuous binder of MHC Class II alleles.

The T cells activated by the APL may be cross reactive with the unmodified or wild type epitope.

A method of identifying a peptide derivative capable of stimulating an immune response as described herein may comprise the step of modifying the peptide structure to produce an APL with altered immunomodulatory properties as described herein.

Modifying the peptide may comprise modifying, substituting, adding or deleting one or more amino acids. Modifications which may be found in peptide derivatives are described herein.

For example, modifying a peptide may comprise systematically altering one or more amino acids in the peptide, e.g. substituting each amino acid in turn. For example, an initial screen may use an alanine scan to prepare a set of peptide derivatives from a starting peptide, each derivative being substituted with an alanine at a single position (11).

Modifying a peptide may comprise adding 1, 2, or 3 (or more) amino acids at the N-terminal end, the C-terminal end, or at both N-terminal and the C-terminal end.

Modification may be at an amino acid within any of SEQ ID NOS:1-913. Alternatively, modification may be at an amino acid in a region flanking any of these sequences, such as the N-terminal and/or C-terminal 1, 2, 3, 4, 5 or 6 amino acids. For example, one or more additional amino acids may be added, substituted or chemically modified at the N-terminal and/or C-terminal region of an epitope. Preferably one or more basic amino acids is included at the C-terminal end of a peptide.

Binding core 9-mers of class II DR epitopes have a general pattern of amino acid side chains important in binding to the MHC and important for binding of the MHC/peptide complex to the T-cell receptor. For a typical peptide epitope, alterations of residues P1, P4, P6 or P9 can alter peptide binding strength to MHC alleles while alterations of P2, P3, P5, P7 and P8 can alter the interactions of MHC/peptide complex with T-cell receptors. Altering the strength of binding of the MHC/peptide complex to the T-cell receptor is known to have the ability to change the fate of the original T-cell receptor clone as to cytokine polarization and/or interact with structurally related T-cell receptor clones not induced by the original peptide.

Candidate APL(s) may be assessed for binding to MHC Class II molecules, in particular HLA Class II molecules such as HLA-DR alleles. Typically an APL is tested for binding to HLA DR alleles which occur at a frequency of at least 40% in the European-American population, for example at least 50%, 60%, 70%, 80% or 90% in the population. Preferably an APL is tested for binding to at least 2, 3, 4, 5, 6, 7, 8, 9, 10, or 11 of the alleles in FIG. 25 (and optionally also to the HLA DR*1401 allele).

Preferably an APL exhibits substantially similar or improved binding compared to the unaltered peptide. Preferably an APL shows promiscuous binding to HLA Class II molecules as described herein.

MHC binding may be assessed using in silico screening. Typically in silico screening, such as the ProPred software described herein, comprises use of virtual HLA Class II matrices. Additionally or alternatively, MHC binding may be assessed using an in vitro binding assay, such as the ProImmune REVEAL® assay described herein.

Candidate APL(s) may be subject to testing in a T cell proliferation assay, and/or in cytokine secretion assays (e.g. assaying for IFNγ or IL-4 production) to determine the nature of the T cell response to the APL. For example, epitope specific T-cell lines and clones can be isolated from sensitized allergic donors. An APL modified from the native sequence may cross-react with the original clones induced by the native peptide and/or it may induce new T-cell receptor clones. Using an original line or clone induced by the native epitope for testing with APLs allows precise characterization of proliferation/cytokine pattern changes on the original population of clones due to amino acid changes in the peptide. Specific APLs that exhibit the desired properties can be tested for effects on whole TCR populations from the targeted patient population.

APLs selected through in vitro validation assays such as those described above may be tested in animal models or patient populations for therapeutic effects on fungal allergy or fungal infection as described herein.

This process of modification of the peptide and testing may be repeated a number of times, as desired, until a peptide having the desired effect, or level of effect, on T cell proliferation and/or cytokine secretion is identified.

In one aspect a peptide derivative herein refers to an APL of any one of SEQ ID NOs:1-913.

### Peptide Solubility

For some applications it is desirable for the peptide to be soluble in a liquid, e.g. water, saline solution or another pharmaceutically acceptable liquid carrier. Some hydrophobic peptides may first be dissolved in DMSO or other solvents and diluted into aqueous solution. Where the hydrophobic character of the peptide prevents such an approach the peptide may be modified to improve solubility. Modification of the peptide may be achieved in several ways well known to one of skill in the art, including the following.

One type of modification involves alteration of the peptide amino acid sequence to provide a peptide derivative in which one or more hydrophobic amino acids are substituted with amino acids of moderate or low hydrophobicity or with charged or uncharged polar amino acids.

Another type of modification involves modification of the N- and/or C-terminal ends of the peptide. Peptide derivatives may be provided in which the N-terminus is free and charged (NH₂—) or blocked with an acetyl group (AC—) or with Biotin. The C-terminus may also be free and charged (—COOH) or blocked (—CONH₂).

Another type of modification involves addition of one, two or three amino acids to the N- and/or C-terminus of the peptide to provide a longer peptide derivative. The additional amino acids may be any amino acids. In preferred embodiments the additional amino acids are chosen from the amino acids adjacent the N- or C-terminus of the peptide sequence as found in the protein amino acid sequence from which the peptide is derived. However, these may be modified to increase solubility.

Following modification to provide a peptide derivative the peptide derivative would be tested for retention of biological activity and for improvement in solubility.

### Sequence Identity

Aspects of the invention concern compounds which are isolated peptides/polypeptides comprising an amino acid sequence having a sequence identity of at least 60% with a given sequence. Alternatively, this identity may be any one of 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity.

Percentage (%) sequence identity is defined as the percentage of amino acid residues in a candidate sequence that are identical with residues in the given listed sequence (referred to by the SEQ ID NO.) after aligning the sequences and introducing gaps if necessary, to achieve the maximum sequence identity, and not considering any conservative substitutions as part of the sequence identity. Sequence identity is preferably calculated over the entire length of the respective sequences.

Alignment for purposes of determining percent amino acid sequence identity can be achieved in various ways known to a person of skill in the art, for instance, using publicly available computer software such as ClustalW 1.82. T-coffee or Megalign (DNASTAR) software. When using such software, the default parameters, e.g. for gap penalty and extension penalty, are preferably used. The default parameters of ClustalW 1.82 are: Protein Gap Open Penalty=10.0, Protein Gap Extension Penalty=0.2, Protein matrix=Gonnet, Protein/DNA ENDGAP=−1, Protein/DNA GAPDIST=4.

Identity of nucleic acid sequences may be determined in a similar manner involving aligning the sequences and introducing gaps if necessary, to achieve the maximum sequence identity, and calculating sequence identity over the entire length of the respective sequences.

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE FIGURES

Embodiments and experiments illustrating the principles of the invention will now be discussed with reference to the accompanying figures in which:

FIG. 1. Table of REVEAL® scores and stability indices for SEQ ID NOs:65, 112, 928, 127, 142, 157, 172, 187, 929, 202, 217, 255, 285, 300, 930, 324, 339, 354, 384, 414, 444, 459, 474, 489 against DRB*0101.

FIG. 2. Table of REVEAL® scores and stability indices for SEQ ID NOs:65, 112, 928, 127, 142, 157, 172, 187, 929, 202, 217, 255, 285, 300, 930, 324, 339, 354, 384, 414, 444, 459, 474, 489 against DRB*1501.

FIG. 3. Table of REVEAL® scores and stability indices for SEQ ID NOs:65, 112, 928, 127, 142, 157, 172, 187, 929, 202, 217, 255, 285, 300, 930, 324, 339, 354, 384, 414, 444, 459, 474, 489 against DRB*0301.

FIG. 4. Table of REVEAL® scores and stability indices for SEQ ID NOs:65, 112, 928, 127, 142, 157, 172, 187, 929, 202, 217, 255, 285, 300, 930, 324, 339, 354, 384, 414, 444, 459, 474, 489 against DRB*0401.

FIG. 5. Table of REVEAL® scores and stability indices for SEQ ID NOs:65, 112, 928, 127, 142, 157, 172, 187, 929, 202, 217, 255, 285, 300, 930, 324, 339, 354, 384, 414, 444, 459, 474, 489 against DRB*1101.

FIG. 6. Table of REVEAL® scores and stability indices for SEQ ID NOs:65, 112, 928, 127, 142, 157, 172, 187, 929, 202, 217, 255, 285, 300, 930, 324, 339, 354, 384, 414, 444, 459, 474, 489 against DRB*1301.

FIG. 7. Table of REVEAL® scores and stability indices for SEQ ID NOs:65, 112, 928, 127, 142, 157, 172, 187, 929, 202, 217, 255, 285, 300, 930, 324, 339, 354, 384, 414, 444, 459, 474, 489 against DRB*0701.

FIG. 8. Table of Pan-Allele REVEAL® binding assay data for SEQ ID NOs:65, 112, 928, 127, 142, 157, 172, 187, 929, 202, 217, 255, 285, 300, 930, 324, 339, 354, 384, 414, 444, 459, 474, 489 (shown in table as Peptide ID Nos1-24).

FIG. 9. Graph showing Pan-Allele REVEAL® binding assay data for SEQ ID NOs:65, 112, 928, 127, 142, 157, 172, 187, 929, 202, 217, 255, 285, 300, 930, 324, 339, 354, 384, 414, 444, 459, 474, 489 (shown in table as Peptide Sequences 1-24).

FIG. 10. Table of Pan-Allele REVEAL® Stability Index data for SEQ ID NOs:65, 112, 928, 127, 142, 157, 172, 187, 929, 202, 217, 255, 285, 300, 930, 324, 339, 354, 384, 414, 444, 459, 474, 489 (shown in table as Peptide ID Nos1-24).

FIG. 11. Graph showing Pan-Allele REVEAL® Stability data for SEQ ID NOs:65, 112, 928, 127, 142, 157, 172, 187, 929, 202, 217, 255, 285, 300, 930, 324, 339, 354, 384, 414, 444, 459, 474, 489 (shown in table as Peptide Sequences 1-24).

FIG. 12. Table of REVEAL® scores and stability indices for SEQ ID NOs:504, 519, 534, 931, 549, 564, 579, 609, 639, 932, 654, 688, 703, 718, 730, 760, 790, 820, 835, 850, 869, 884, 893, 905 against DRB*0101.

FIG. 13. Table of REVEAL® scores and stability indices for SEQ ID NOs:504, 519, 534, 931, 549, 564, 579, 609, 639, 932, 654, 688, 703, 718, 730, 760, 790, 820, 835, 850, 869, 884, 893, 905 against DRB*1501.

FIG. 14. Table of REVEAL® scores and stability indices for SEQ ID NOs:504, 519, 534, 931, 549, 564, 579, 609, 639, 932, 654, 688, 703, 718, 730, 760, 790, 820, 835, 850, 869, 884, 893, 905 against DRB*0301.

FIG. 15. Table of REVEAL® scores and stability indices for SEQ ID NOs:504, 519, 534, 931, 549, 564, 579, 609, 639, 932, 654, 688, 703, 718, 730, 760, 790, 820, 835, 850, 869, 884, 893, 905 against DRB*0401.

FIG. 16. Table of REVEAL® scores and stability indices for SEQ ID NOs:504, 519, 534, 931, 549, 564, 579, 609, 639, 932, 654, 688, 703, 718, 730, 760, 790, 820, 835, 850, 869, 884, 893, 905 against DRB*0701.

FIG. 17. Table of Pan-Allele REVEAL® binding assay data for SEQ ID NOs:504, 519, 534, 931, 549, 564, 579, 609, 639, 932, 654, 688, 703, 718, 730, 760, 790, 820, 835, 850, 869, 884, 893, 905 (shown in table as Peptide ID Nos1-24).

FIG. 18. Graph showing Pan-Allele REVEAL® binding assay data for SEQ ID NOs:504, 519, 534, 931, 549, 564, 579, 609, 639, 932, 654, 688, 703, 718, 730, 760, 790, 820, 835, 850, 869, 884, 893, 905 (shown in table as Peptide Sequences 1-24).

FIG. 19. Table of Pan-Allele REVEAL® Stability Index data for SEQ ID NOs:504, 519, 534, 931, 549, 564, 579, 609, 639, 932, 654, 688, 703, 718, 730, 760, 790, 820, 835, 850, 869, 884, 893, 905 (shown in table as Peptide ID Nos1-24).

FIG. 20. Graph showing Pan-Allele REVEAL® Stability data for SEQ ID NOs:504, 519, 534, 931, 549, 564, 579, 609, 639, 932, 654, 688, 703, 718, 730, 760, 790, 820, 835, 850, 869, 884, 893, 905 (shown in table as Peptide Sequences 1-24).

FIG. 21. Table showing peptides subjected to ProImmune REVEAL® assay (SEQ ID NOs:65, 112, 928, 127, 142, 157, 172, 187, 929, 202, 217, 255, 285, 300, 930, 324, 339, 354, 384, 414, 444, 459, 474, 489). Position of the 9mer epitope derived from ProPred in the 15mer tested shown in red, M/L and C/V substitutions are highlighted in light blue. The parent allergen polypeptide is indicated, e.g. Alt a 1, and the start and stop positions of the 9mer in the parent allergen polypeptide sequence is also indicated. Physicial properties of the 15mer (molecular weight, net charge, average hydrophilicity) is provided. Negative binders in the ProImmune REVEAL® assay are shaded in the “15mer ALG Code” column (34.2, 36.2 and L6-1 were negative). Note that the 15mer of SEQ ID NO:65 starts at the Alt a 1 N-terminus and the 15mer of SEQ ID NO:300 starts at the Alt a 1 C-terminus.

FIG. 22. Table showing peptides subjected to ProImmune REVEAL® assay (SEQ ID NOs:504, 519, 534, 931, 549, 564, 579, 609, 639, 932, 654, 688, 703, 718, 730, 760, 790, 820, 835, 850, 869, 884, 893, 905). Position of the 9mer epitope derived from ProPred in the 15mer tested shown in red, M/L and ON substitutions are highlighted in light blue. The parent allergen polypeptide is indicated, e.g. Alt a 3, and the start and stop positions of the 9mer in the parent allergen polypeptide sequence is also indicated. Physicial properties of the 15mer (molecular weight, net charge, average hydrophilicity) is provided. Negative binders in the ProImmune REVEAL® assay are shaded in the “15mer ALG Code” column (54.2, and 49.2.1 were negative). Note that the 15mer and 9mer of SEQ ID NO:730 starts at the Alt a 5 N-terminus and the 15mer and 9mer of SEQ ID NO:893 starts at the Alt a 10 C-terminus.

FIG. 23. Table showing 9mer epitope sequences identified by ProPred analysis in Alt a 1.

FIG. 24. Table of conservative amino acid modifications indicating amino acid modifications that may be made to peptides of the invention in order to increase peptide resistance to degradation.

FIG. 25. Table of top 11 DRB1 alleles used in ProPred search. Alleles are shown by percentage population frequency present in European Americans.

FIG. 26. Table showing alleles for which 9mer epitopes were identified by ProPred analysis for each of Alt a 1, Alt a 3, Alt a 4, Alt a 5, Alt a 6, Alt a 7, Alt a 8, and Alt a 10.

FIG. 27. Table showing SEQ ID NOs:933-1163.

FIG. 28. Table showing ProPred predicted 9mer sequences and predicted DRB allele binding selected under threshold setting 3 (high stringency).

FIG. 29. Graph showing IL-4 induction in individual patients in response to individual Alt a 1 soluble peptides and the Alt a 8 peptide 47.2.1.

FIG. 30. Graph showing IL-4 induction in individual patients in response to individual Alt a 1 DMSO peptides and the Alt a 8 peptide 47.2.1.

FIG. 31. Graph showing IL-4 induction in individual patients in response to individual Alt a 3, Alt a 4, Alt a 6, Alt a 7, Alt a 8, and Alt a 10 peptides.

FIG. 32. Graph showing IL-4 induction in individual patients in response to combination preparations Alt a 1 Sol, Alt a 1 DMSO, Alt a Oth Sol, Alt a Oth DMSO.

### DETAILED DESCRIPTION OF THE INVENTION

The details of one or more embodiments of the invention are set forth in the accompanying description below including specific details of the best mode contemplated by the inventors for carrying out the invention, by way of example. It will be apparent to one skilled in the art that the present invention may be practiced without limitation to these specific details.

Treatment of allergic disease with peptide epitopes derived from or corresponding to peptide fragments or protein allergens offers substantial advantages over treatment with full length allergen protein molecules because of the reduced potential for cross-linking IgE bound to the surface of mast cells and basophils (12).

MHC class II genes encode cell surface glycoproteins having structural similarity to MHC Class 1 molecules but expressed only on Antigen Presenting Cells (APC). In conjunction with antigenic protein allergen fragments MHC class II proteins are recognised by T-helper (CD4+) cells. Therefore MHC class II molecules are involved in nearly all antigen responses. In humans MHC molecules are referred to as Human Leukocyte Antigens (HLA) encoded by genes HLA-DM, HLA-DO, HLA-DP, HLA-DQ and HLA-DR.

A T cell epitope is the basic unit recognised by a T cell receptor. T cell epitopes are believed to be involved in the initiation and perpetuation of the immune response to an antigen such as a protein allergen which is responsible for the clinical symptoms of allergy. These T cell epitopes are thought to trigger early immune response events at the level of the T helper cell by binding to an appropriate HLA molecule on the surface of an antigen presenting cell and stimulating the relevant T cell subpopulation. These events lead to T cell proliferation, cytokine secretion, local inflammatory reactions, recruitment of additional immune cells to the site, and activation of the B cell cascade leading to production of antibodies.

A therapeutic/prophylactic treatment regimen in accordance with the invention (which results in prevention of, or delay in, the onset of disease symptoms caused by an offending antigen or results in reduction, slowed progression, or alleviation of symptoms caused by an offending antigen i.e. down regulation of an antigen specific immune response) comprises administration, in non-immunogenic form (e.g. without adjuvant) of a pharmaceutical composition of the invention comprising at least one isolated peptide which may be derived from a protein antigen responsible for the disease condition being treated. While not intending to be limited to any theory, it is believed that administration of a therapeutic composition of the invention may: (i) cause T cell non responsiveness of appropriate T cell subpopulations such that they become unresponsive to the offending antigen and do not participate in stimulating an immune response upon exposure to the offending protein antigen (i.e. via anergy or apoptosis); (ii) modify the cytokine secretion profile as compared with exposure to the naturally occurring offending antigen (e.g. result in a decrease of IL-4 and/or an increase in INF-γ); (iii) cause T cell subpopulations which normally participate in the response to the offending antigen to be drawn away from the sites of normal exposure (e.g. nasal mucosa, skin and lung for allergy) towards the sites of administration of the composition (this redistribution of T cell subpopulations may ameliorate or reduce the ability of an individual's immune system to stimulate the usual immune response at the site of normal exposure to the offending antigen, resulting in diminution in allergic symptoms); or (iv) cause induction of T suppressor cells, e.g. induction of allergen specific IL-10 producing Treg CD⁺ T-cells.

It is preferred that peptides according to the present invention do not bind immunoglobulin E (IgE) or bind IgE to a substantially lesser extent (e.g. at least 100-fold less binding and more preferably at least 1,000-fold less binding) than the protein allergen from which the peptide has been identified. Major complications of standard immunotherapy include IgE-mediated responses such as anaphylaxis. Thus for example, a method of identifying a peptide derivative may comprise assaying for binding to IgE, and selecting a peptide with the required binding as above.

HLA-DR molecules have a wide range of structures owing to the HLA-DR molecule being encoded by several loci with several genes at each locus. This leads to a large number and diversity of HLA-DR alleles and variants with a wide range of allele frequency between ethnic and geographical populations. Additionally the HLA-DR allele population is constantly evolving.

Some allergen fragments are only bound by one or two HLA-DR molecules and hence are considered MHC restricted. Use of such fragments as vaccines can only be expected to benefit those patients expressing the required allele and can therefore severely limit the use of the vaccine to treat the majority of a patient population that has a wide diversity of HLA-DR alleles. Some allergen fragments are bound by many HLA-DR alleles and therefore may provide the basis of vaccines capable of treating a much larger patient population.

Genomic analysis of allergen genes in Alternaria alternata has identified a number of protein allergens and potential allergens via identity to allergens from other fungal species (6-7) including Alt a 1, Alt a 3, Alt a 4, Alt a 5, Alt a 6, Alt a 7, Alt a 8, Alt a 10. These sequences were subject to analysis for epitopes exhibiting promiscuous binding to HLA-DR alleles.

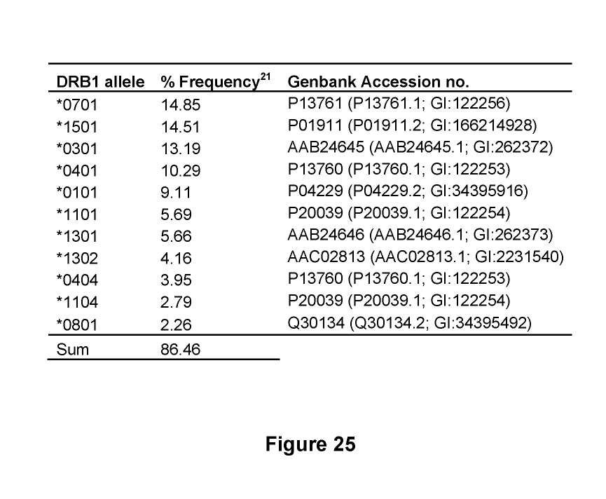

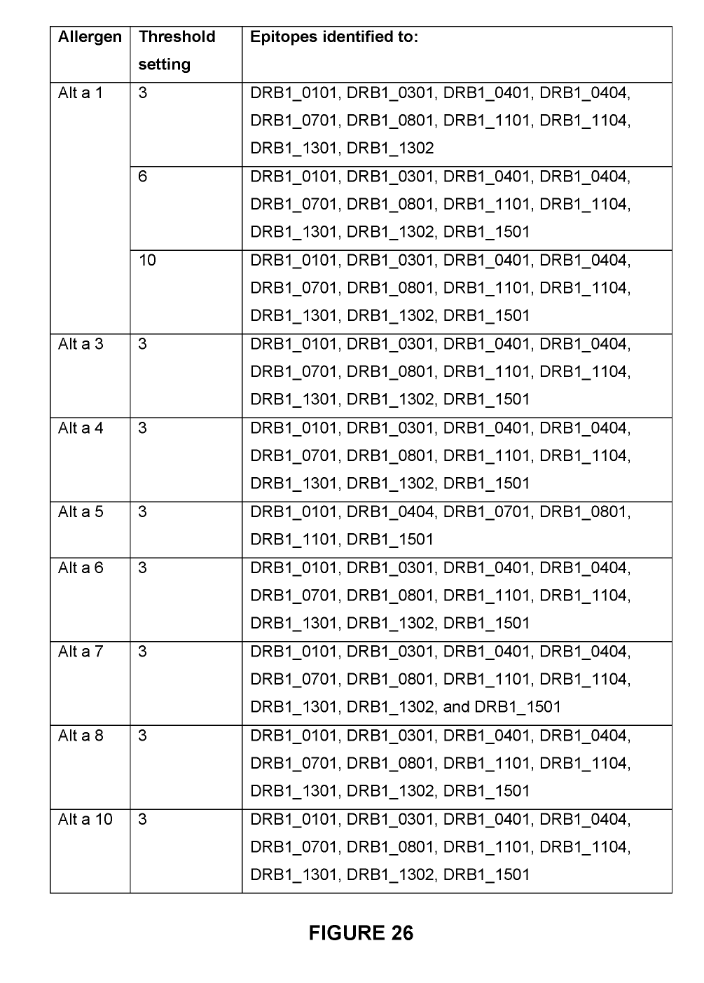

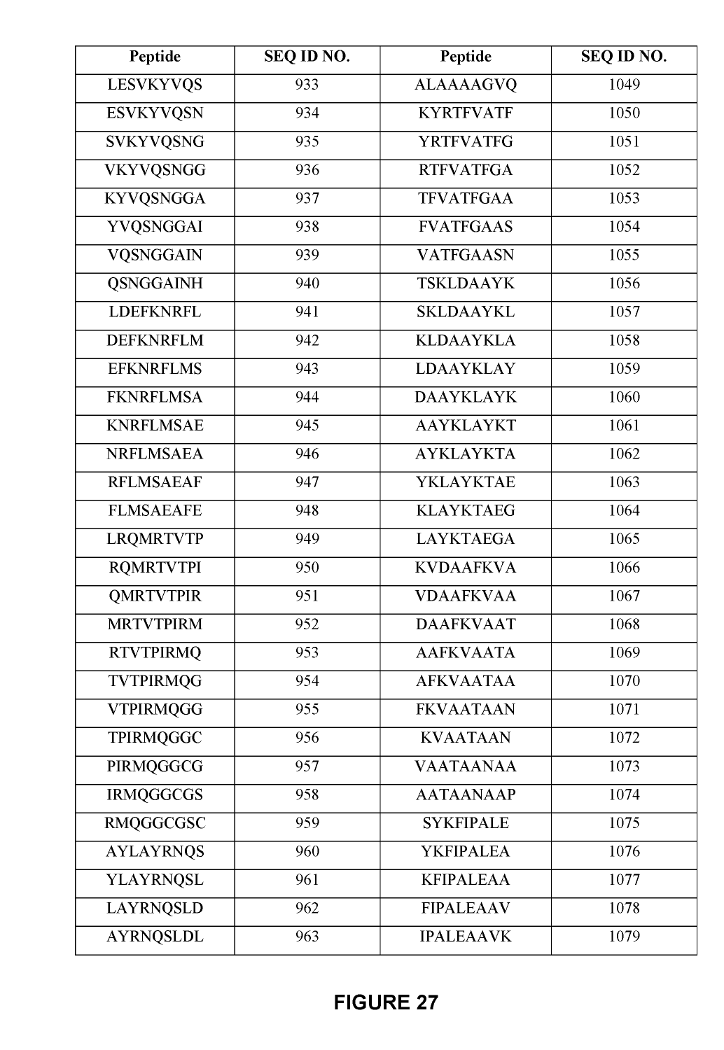

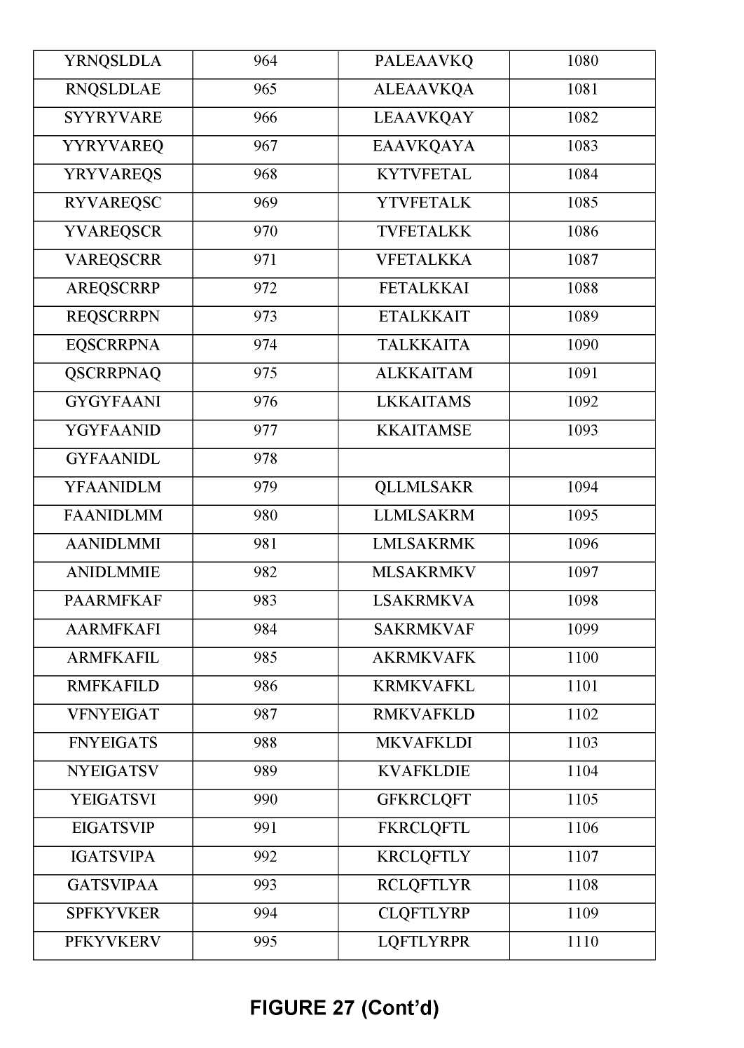

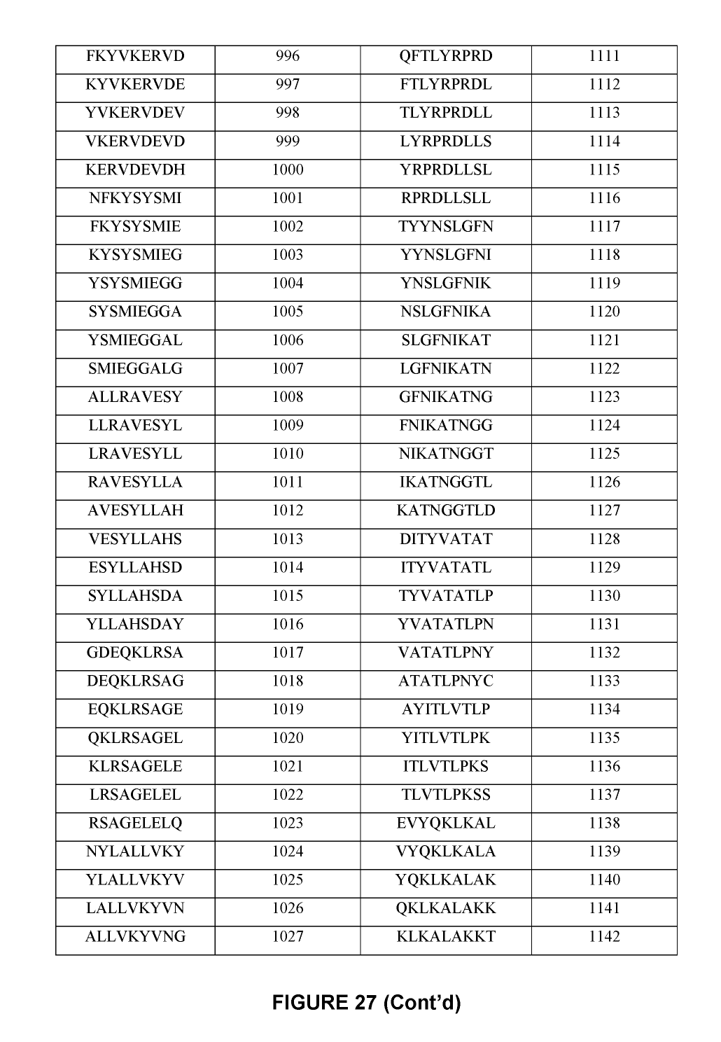

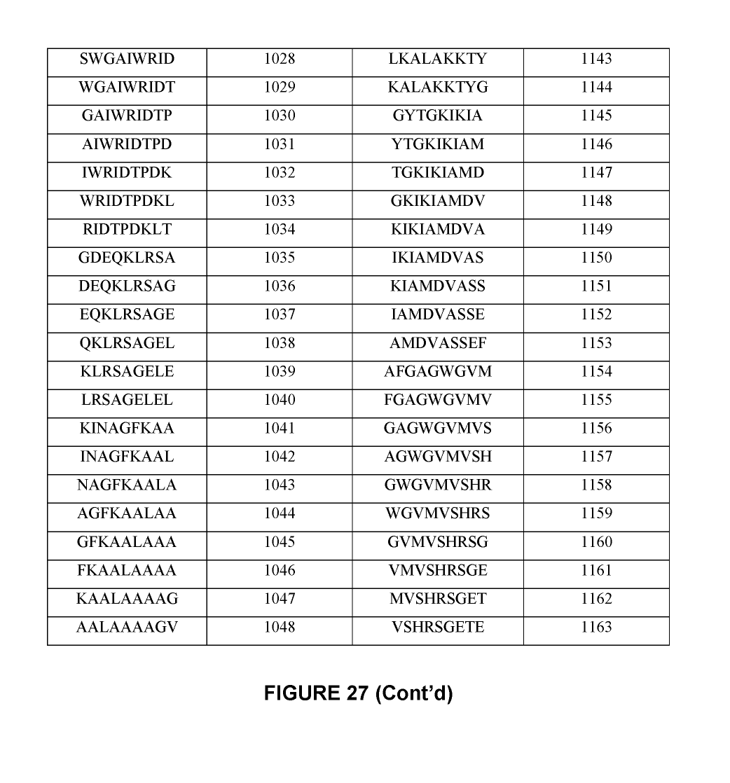

The inventors first identified peptides from these protein allergens predicted to be T cell epitopes to one or more of the alleles listed in FIG. 25.

This first stage in the identification of peptides as candidate T cell epitopes for in vivo validation as vaccines for fungal allergy was performed by use of virtual HLA class II matrices. In such a system a matrix-based algorithm is used to extract all possible peptide frames from a given protein allergen sequence. Subsequently, the corresponding position- and amino acid-specific matrix values are assigned to each residue of these peptide frames. Finally, the sum of these matrix values is determined for each frame. The resulting numerical values (peptide scores) correlate with the binding affinity of HLA-DR ligands (16). One such method of predicting T cell epitopes is provided by the TEPITOPE software (9, 10, 16).

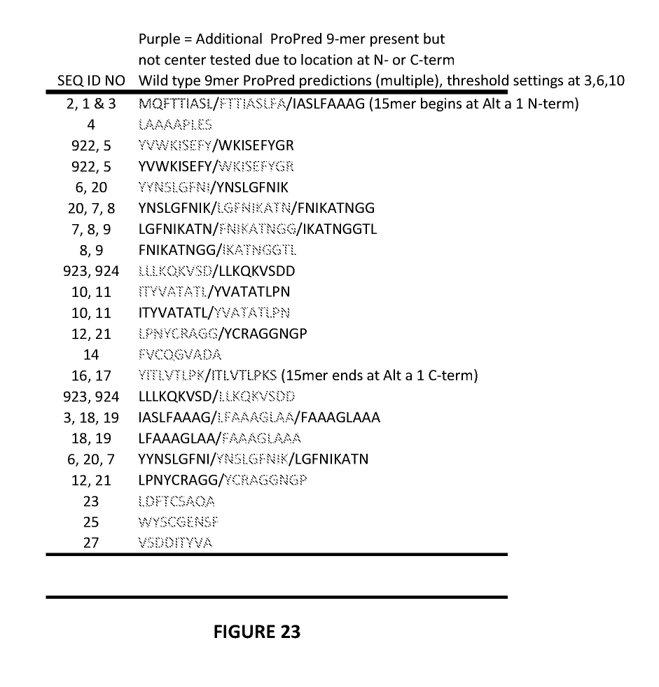

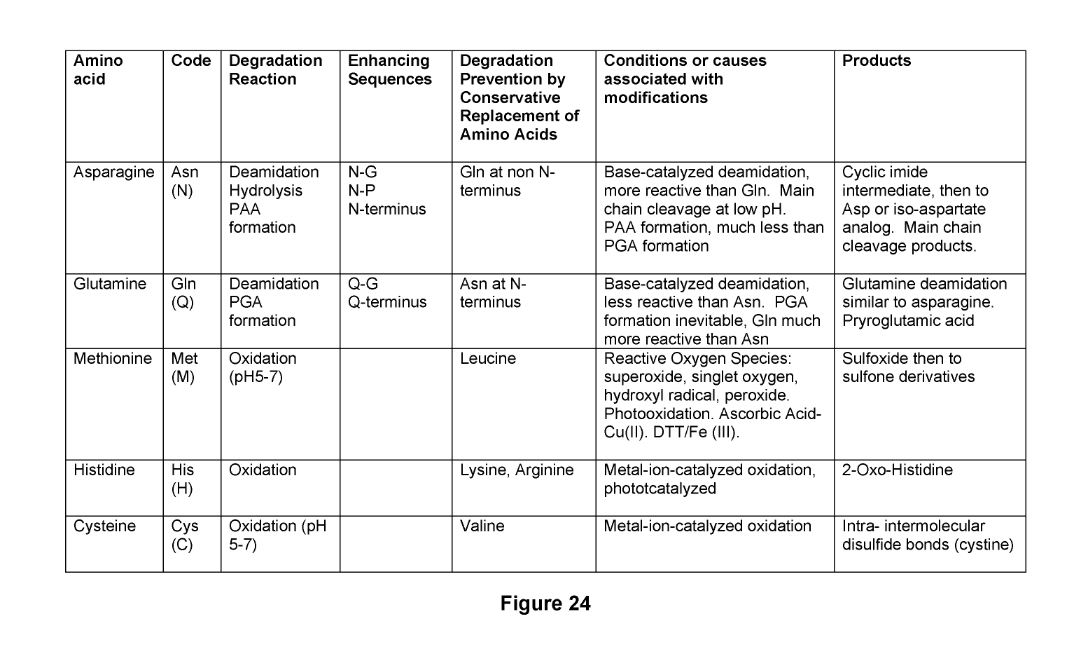

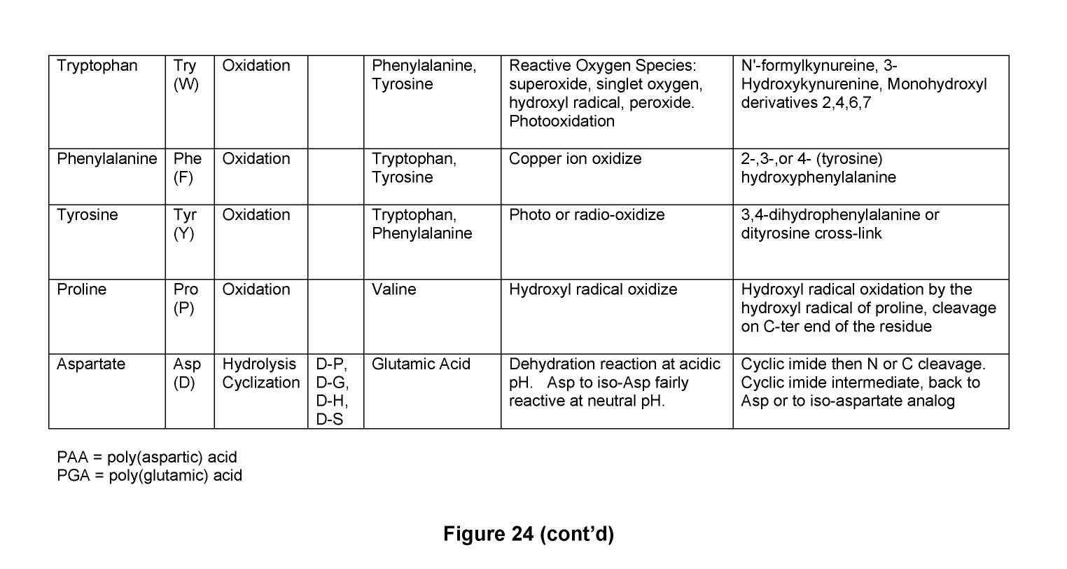

In the embodiments described here the inventors used the ProPred epitope prediction software (8) [http://www.imtech.res.in/raghava/propred/index.html], running experiments at levels 3, 6 and 10 (3 is the most stringent). This computes the binding strength to each HLA-DR allele of all peptide frames in the selected allergen sequence. The ProPred software contains 51 HLA-DR alleles. Potential promiscuous binders were selected from fungal protein allergens by identifying 9mer epitope sequences predicted by ProPred to bind more than one of the HLA-DR alleles listed in FIG. 25. The peptide epitopes identified using the ProPred software are shown in FIG. 23.

Some of the epitopes identified contain methionine or cysteine residues. When synthesising peptides for in vitro testing peptide derivatives were prepared in which Methionine residues were replaced with Leucine and Cysteine residues with Valine (M/L and C/V derivatives).

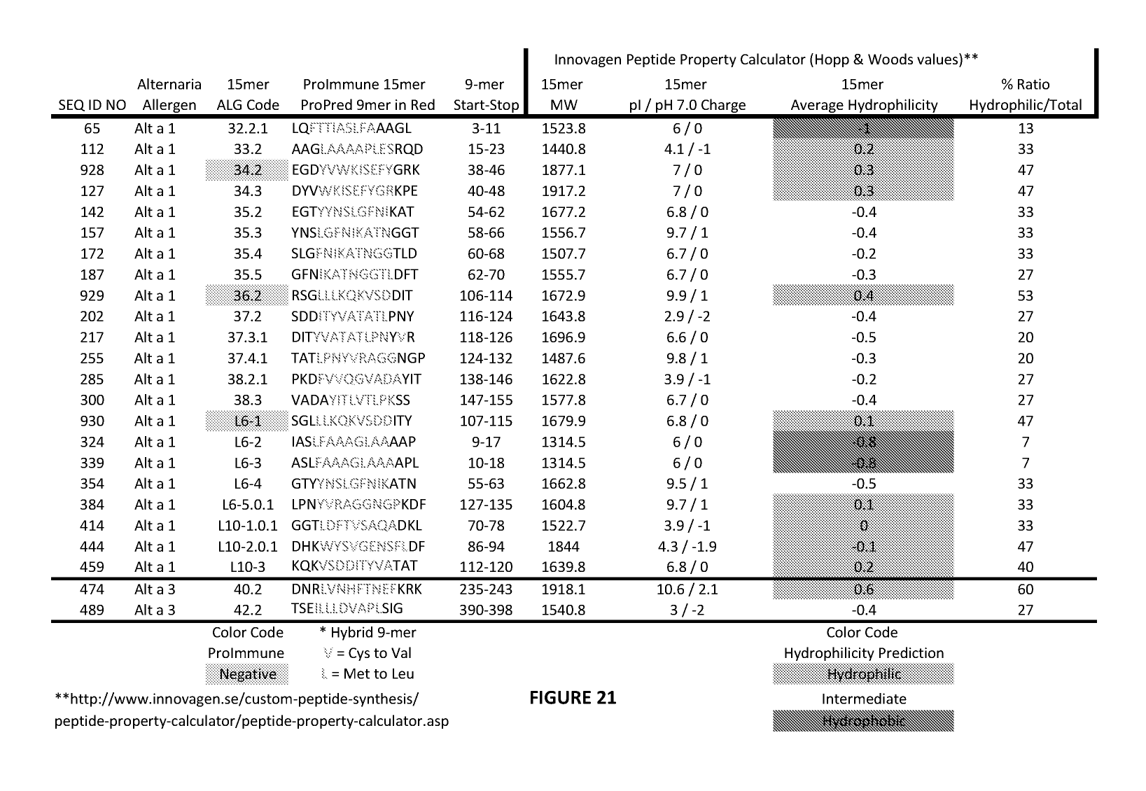

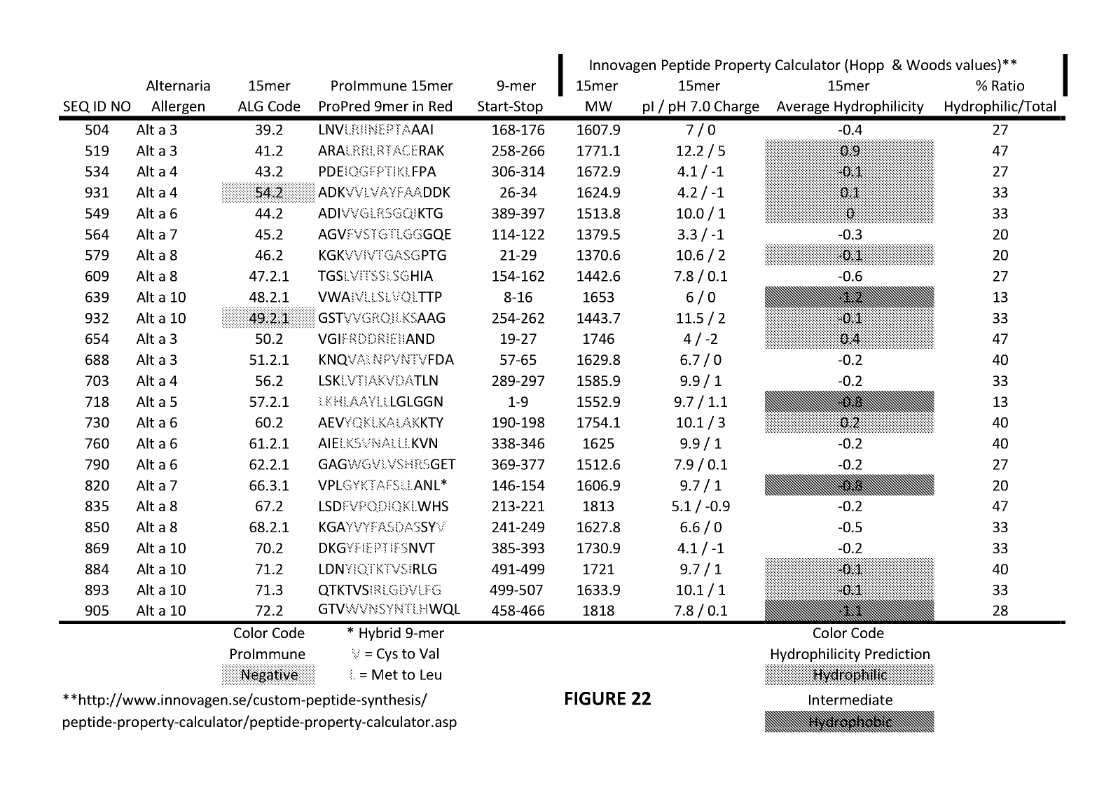

The promiscuous epitopes identified using the ProPred software were used to synthesise 15mer peptides comprising the core 9mer sequence. The additional flanking amino acids normally correspond to the amino acids flanking the 9mer sequence in the wild type fungal protein sequence from which the 9mer sequence is derived. For example, most 15mers were prepared by synthesising a 15 amino acid sequence of the wild type allergen in which the 9mer is centred in the synthesised sequence, being flanked by 3 amino acids at the N-terminal end, and 3 additional amino acids at the C-terminal end. In some cases the 9mer epitope sequence occurs close to the N- or C-terminal end of the wild type allergen polypeptide. In these cases, the 9mer was not centred in the synthesised 15mer. The 15mers are shown in FIGS. 21 and 22. In some 15mers, the synthesised peptide differs from the amino acid sequence of the polypeptide allergen in that Methionine residues were replaced with Leucine and Cysteine residues with Valine (M/L and C/V derivatives).

The 15mer peptides were tested for binding to class II HLA alleles using the ProImmune REVEAL® Class II HLA-Peptide Binding Assay (http://www.proimmune.com/ecommerce/page.php?page=reveal class2) (26). Potential T-cell epitopes predicted by software can be screened by the experimental determination of their binding to specific MHC molecules (Panigada et al. Infec. Immun. 2002. 70:79-85). A commercial fee-for-service to measure the binding of peptides to MHC molecules has been developed by ProImmune Ltd (Oxford UK) and has been used to predict epitopes to MHC Class I (Westrop et al. J. Immunol. Methods. 2009. 341:76-85) and MHC Class II DR (Muixi et al. J. Immunol. 2008. 181:795-807) alleles.

The ProImmune Class II REVEAL® rapid epitope discovery assay is a cell-free in vitro assay that determines binding of a candidate peptide to one or more HLA class II DRB1 alleles compared with a positive and an intermediate control peptide. The assay is a measure of the ability of each peptide to stabilise the MHC-peptide complex. Detection is based on the presence or absence of the native conformation of this MHC-peptide complex. For the standard REVEAL assay the peptides to be tested are first synthesized as 15mers (Module 1: PEPscreen peptide synthesis) followed by incubation with the appropriate MHC protein at two dilutions for 7 days at 10° C. followed by a single “on” point measurement (Module 2: REVEAL® MHC binding assay). Each peptide is given a score relative to the positive control peptide which is a known T cell epitope. Results for an intermediate control peptide are also shown. The intermediate control is a known T cell epitope, or in the case of certain class II alleles, a known epitope for the beta-chain in the allele alpha and beta pair. The score is reported quantitatively as a percentage of the signal generated by the test peptide relative to the positive control peptide. Proimmmune considers peptides that bind ≧15% relative to the control as being “passed”. To further confirm a peptide as an epitope, after the initial “on” point determination the reaction temperature is raised to 37° C. followed by stability measurements at 0 and 24 hrs from which a stability index is calculated by multiplying the estimated half-life by the module 2 REVEAL score (Module 4: Quick check stability assay). The higher the stability index the slower the off-rate and the more stable the epitope.

Of the alleles available for the assay, the inventors selected HLA-DRB1*0101, DRB1*1501, DRB1*0301, DRB1*0401, DRB1*0701, DRB1*1101 and DRB1*1301 for testing of SEQ ID NOs:65, 112, 928, 127, 142, 157, 172, 187, 929, 202, 217, 255, 285, 300, 930, 324, 339, 354, 384, 414, 444, 459, 474, 489 and HLA-DRB1*0101, DRB1*1501, DRB1*0301, DRB1*0401 and DRB1*0701 for testing of SEQ ID NOs:504, 519, 534, 931, 549, 564, 579, 609, 639, 932, 654, 688, 703, 718, 730, 760, 790, 820, 835, 850, 869, 884, 893, 905.

DRB1*1501, DRB1*0301, and DRB1*0701 are the three DRB1 alleles having the highest frequency of occurrence in the European/American population, and together have a total frequency of 42.55% in the population (see FIG. 25).

Thus, as set out in the following Examples the inventors carried out an in silico screen for epitopes amongst known and/or suspected fungal allergens from Alternaria alternata, and arranged in vitro analysis of synthetic peptides containing the identified epitopes to confirm their promiscuous binding character.

### EXAMPLES

### Example 1

### Identification of Predicted Promiscuous T-Cell Epitopes

Predicted T cell epitopes were identified from Alternaria alternata protein allergens Alt a 1, Alt a 3, Alt a 4, Alt a 5, Alt a 6, Alt a 7, Alt a 8, Alt a 10.

The amino acid sequences of the polypeptides were first subject to analysis using the ProPred software (http://www.imtech.res.in/raghava/propred/index.html, ref 8) in order to identify predicted HLA-DR binding peptide epitopes within each protein allergen. In total, ProPred includes 51 HLA-DR alleles against which peptide binding may be assessed. Peptide containing epitopes were selected on the basis of predicted binding to the following alleles: DRB1_{—}0101, DRB1_{—}0301, DRB1_{—}0401, DRB1_{—}0404, DRB1_{—}0701, DRB1_{—}0801, DRB1_{—}1101, DRB1_{—}1104, DRB1_{—}1301, DRB1_{—}1302, and DRB1_1501.

The alleles listed above represent 11 of the 12 most common HLA-DR alleles occurring in European Americans (1401 is not present in the ProPred database). The 11 alleles listed occur in 86.46% of the population (21). Therefore epitopes predicted to bind to 10 or 11 of these alleles can be considered extremely promiscuous HLA-DR binders.

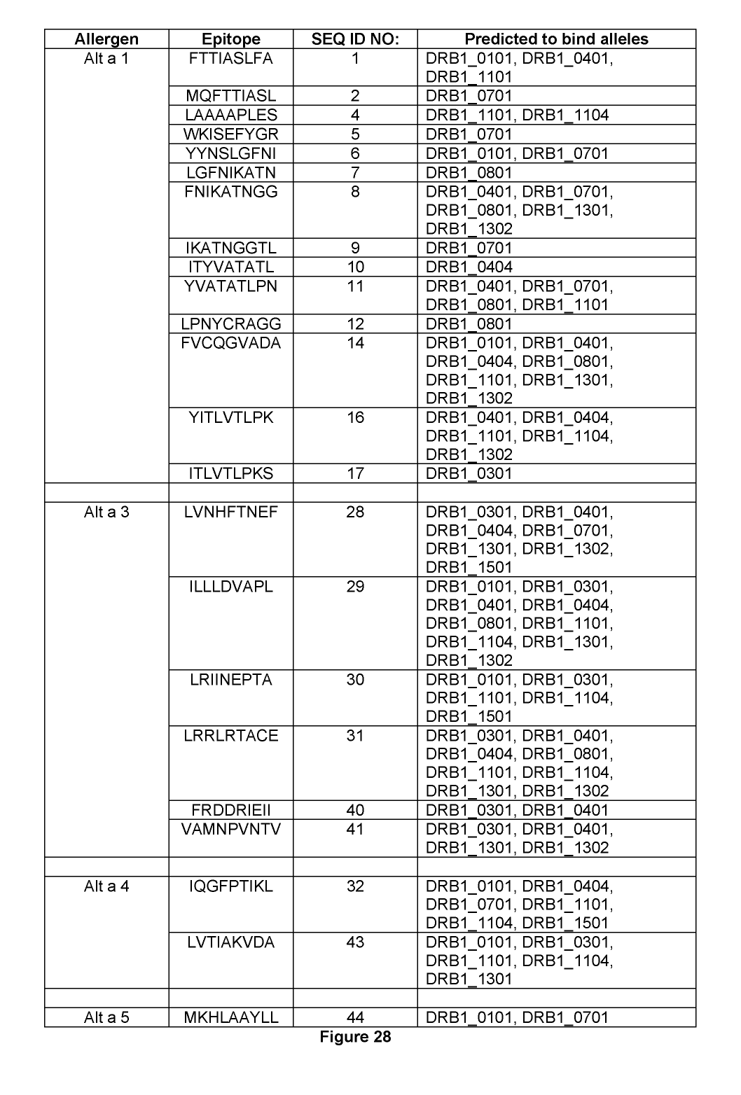

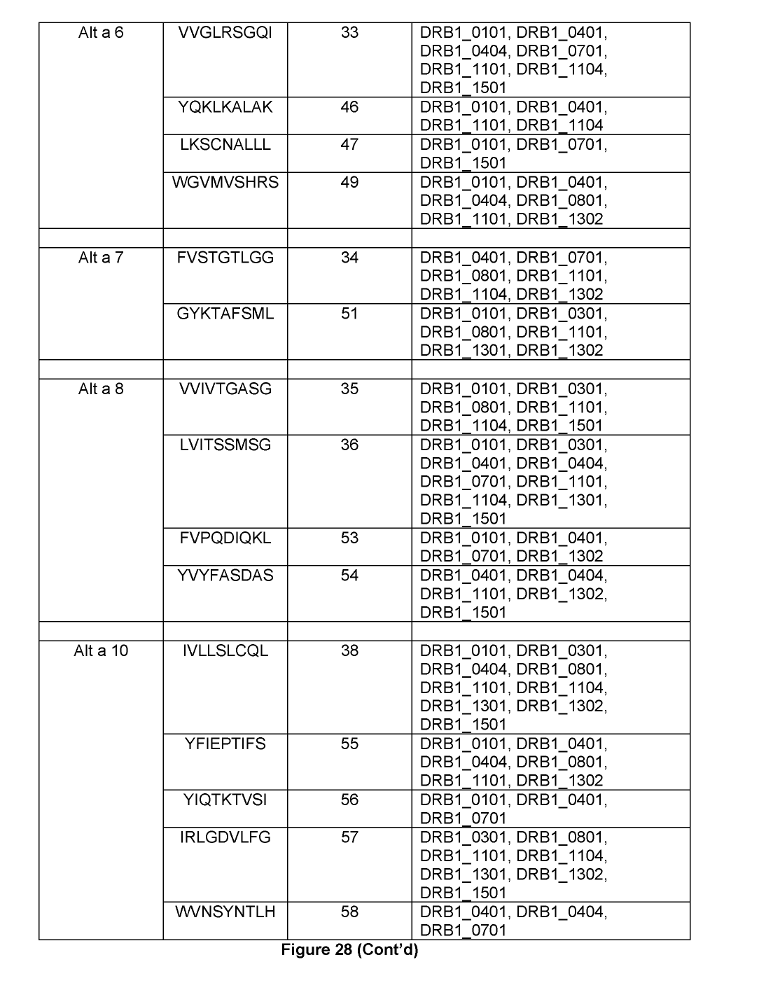

ProPred predictions for all allergens tested were obtained using threshold setting 3 (high stringency). For Alt a 1 additional predictions were obtained using threshold settings 6 and 10. FIG. 28 shows predicted 9mer sequences selected using threshold setting 3 and indicates the DRB alleles predicted to be bound by the 9mer.

Several of the 9mers predicted to be promiscuous epitopes using the ProPred software contained a Methionine residue (M). Methionine is an oxidation-sensitive residue, and is sometimes substituted with a leucine (L) residue, which is less oxidation sensitive. Accordingly, the inventors also devised 9mer derivatives having M/L substitution(s).

Several of the 9mers predicted to be promiscuous epitopes using the ProPred software contained a Cysteine residue (C). The presence of Cysteine residues can lead to low solubility which is undesirable for a candidate therapeutic. Therefore, the inventors also devised 9mer derivatives having ON substitution(s).

Accordingly, using the ProPred data, the inventors identified predicted promiscuous 9mer epitopes from each allergen tested.

### Example 2

### Peptide Design

Peptides containing predicted promiscuous 9mer epitopes identified in Example 1 were synthesised and assayed for T cell binding using the ProImmune REVEAL® assay.

Each synthesised peptide comprised a 15 amino acid (15mer) peptide. In most cases the 9mer epitope identified in Example 1 was centred in the 15mer, such that the 9mer is flanked by 3 amino acids at each of the N- and C-terminal ends. The 9mer epitope and flanking sequences were designed starting from the wild type amino acid sequence of the corresponding 9mer and corresponding flanking sequences in the parent allergen polypeptide such that the peptide has 100% sequence identity with a 15 amino acid sequence within the parent allergen polypeptide. In some cases, the 9mer epitope is located close to the N- or C-terminus of the parent allergen polypeptide amino acid sequence such that less than 3 flanking amino acids exist in the parent allergen polypeptide amino acid sequence. In these cases, e.g. SEQ ID NOs:65, 718 and 893 the 9mer is not centred in the 15mer. Finally, the 15mer sequence was reviewed for the presence of Methionine or Cysteine residues, and M/L and/or ON substitution made.

The peptides so designed and synthesised are listed in FIGS. 21 and 22 as SEQ ID NOs:65, 112, 928, 127, 142, 157, 172, 187, 929, 202, 217, 255, 285, 300, 930, 324, 339, 354, 384, 414, 444, 459, 474, 489 and SEQ ID NOs:504, 519, 534, 931, 549, 564, 579, 609, 639, 932, 654, 688, 703, 718, 730, 760, 790, 820, 835, 850, 869, 884, 893, 905, respectively

The hydrophilicity and net charge was calculated for each candidate peptide using the Innovagen Peptide Property Calculator (Innovagen, Sweden) and are shown in FIGS. 21 and 22. Average hydrophilicty of >−0.1 indicates high peptide solubility in physiological buffers.

The synthesised 15mer sequences were submitted to the ProImmune REVEAL® Class II HLA-Peptide Binding Assay (http://www.proimmune.com/ecommerce/page.php?paqe=reveal class2) (ref 26).

### Example 3

### In Vitro Analysis of Promiscuous Binding to HLA DR Alleles

The REVEAL® Rapid Epitope Discovery System consists of several modules:

Module 1: Custom Peptide Synthesis

The peptides are synthesized in 0.5-2 mg quantities with high average purity. Quality control by MALDI-TOF Mass Spectrometry is carried out on 100% of samples.

Module 2: REVEAL® MHC-Peptide Binding Assay

The high-throughput REVEAL® binding assay determines the ability of each candidate peptide to bind to one or more MHC class II alleles and stabilize the MHC-peptide complex. By comparing the binding to that of high and intermediate affinity T-cell epitopes, immunogenic peptides can be identified. Detection is based on the presence or absence of the native conformation of the MHC-peptide complex.

Each peptide is given a score relative to a positive control peptide, which is a known T-cell epitope, or in the case of certain class II alleles, a known epitope for the beta-chain in the allele alpha and beta pair. The score of the test peptide is given quantitatively as a percentage of the signal generated by the positive control peptide. Assay performance is confirmed by including an intermediate control peptide that is known to bind with weaker affinity to the allele under investigation.

The REVEAL® assay can identify peptides that bind a wide range of DR alleles.

Module 4: REVEAL® Rate Assays

REVEAL® rate assays assess the on- and off-rates for peptide binding to MHC class II molecules. By indicating how long individual epitopes are presented to T-cells they can help identify which candidate sequences can be presented long enough to be good T cell epitopes.

### Quick Check Stability Assay

This assay measures the amount of peptide bound to the allele at time zero and time 24 hours, at 37° C., and gives an indication of the stability of the HLA-peptide complex. Each peptide is given a ‘stability index’, allowing comparison of the relative stability of the different peptide epitopes. The REVEAL® MHC-peptide binding assay reflects the on-rate properties of a peptide more strongly than the stability of the assembled complex. However, when the outcomes of the stability assay and the binding assay are combined, the subsequent data provide more complete information as to whether the peptide could be presented long enough for it to be a good T-cell epitope. This index provides a method for comparing the results of different peptides across the allele being measured.

The synthesised 15mer sequences from Example 2 were submitted to the ProImmune REVEAL® Class II HLA-Peptide Binding Assay (http://www.proimmune.com/ecommerce/page.php?page=reveal class2) (ref 26).

Of the alleles available for the assay, the inventors selected HLA-DRB1*0101, DRB1*1501, DRB1*0301, DRB1*0401, DRB1*0701, DRB1*1101 and DRB1*1301 for testing of SEQ ID NOs:65, 112, 928, 127, 142, 157, 172, 187, 929, 202, 217, 255, 285, 300, 930, 324, 339, 354, 384, 414, 444, 459, 474, 489 and HLA-DRB1*0101, DRB1*1501, DRB1*0301, DRB1*0401 and DRB1*0701 for testing of SEQ ID NOs:504, 519, 534, 931, 549, 564, 579, 609, 639, 932, 654, 688, 703, 718, 730, 760, 790, 820, 835, 850, 869, 884, 893, 905.

DRB1*1501, DRB1*0301, and DRB1*0701 are the three DRB1 alleles having the highest frequency of occurrence in the European/American population, and together have a total frequency of 42.55% in the population (see FIG. 25).

Binding to MHC molecules was compared to that of two known T-cell epitopes: a positive control peptide and an intermediate control peptide with very strong and weaker binding properties, respectively.

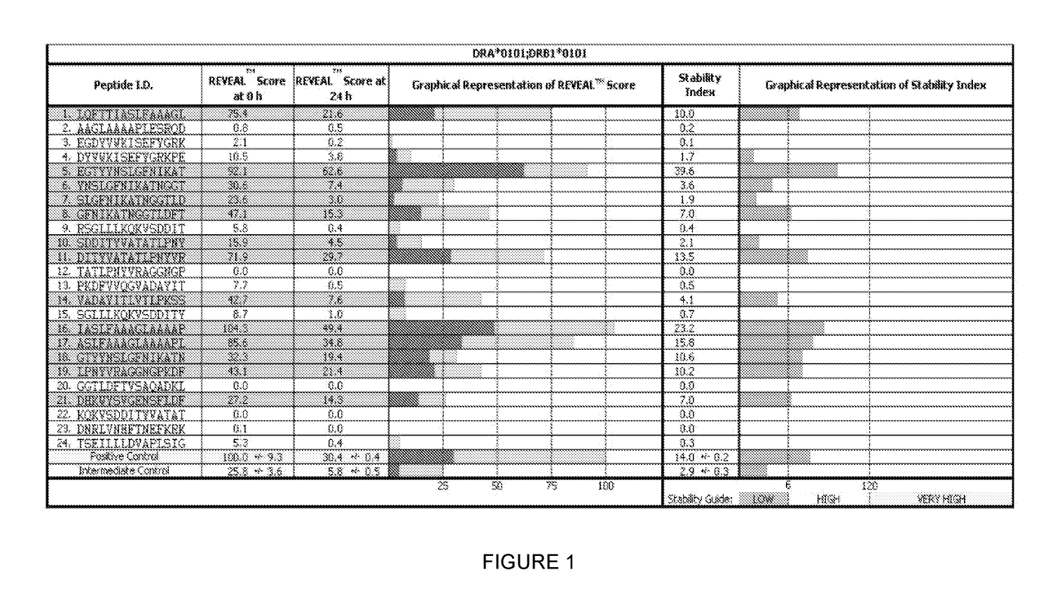

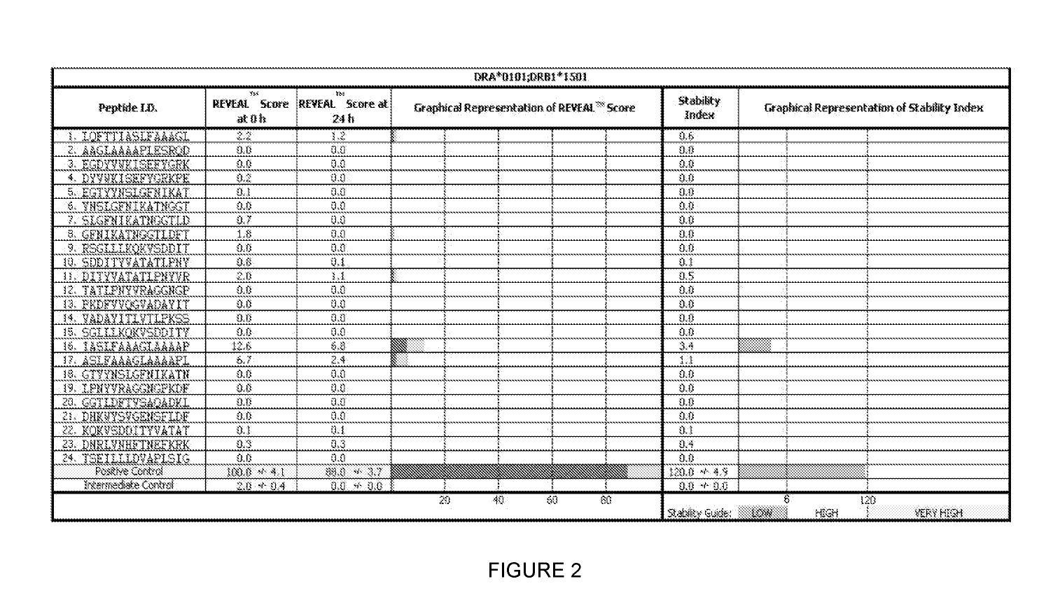

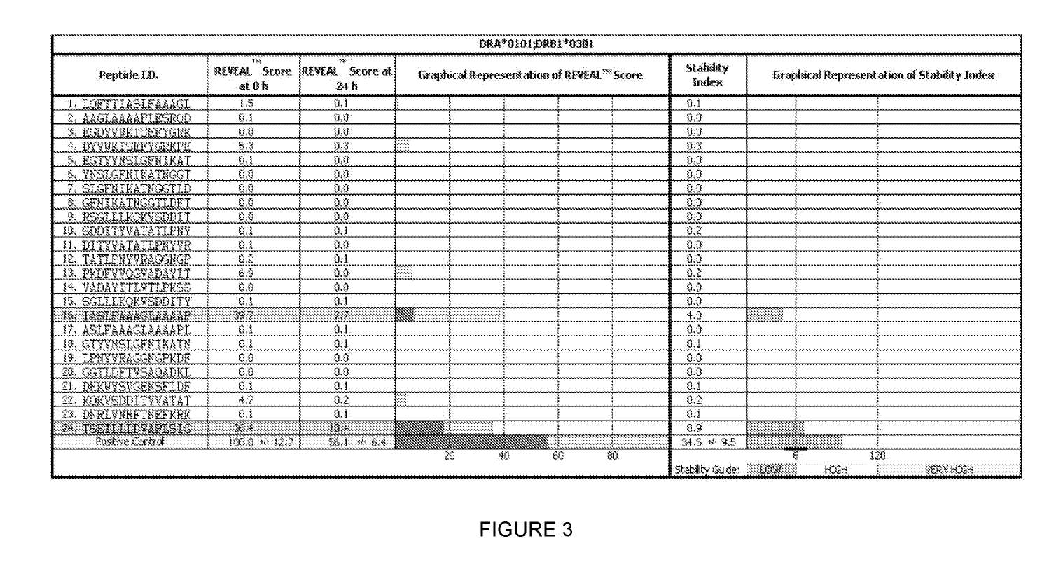

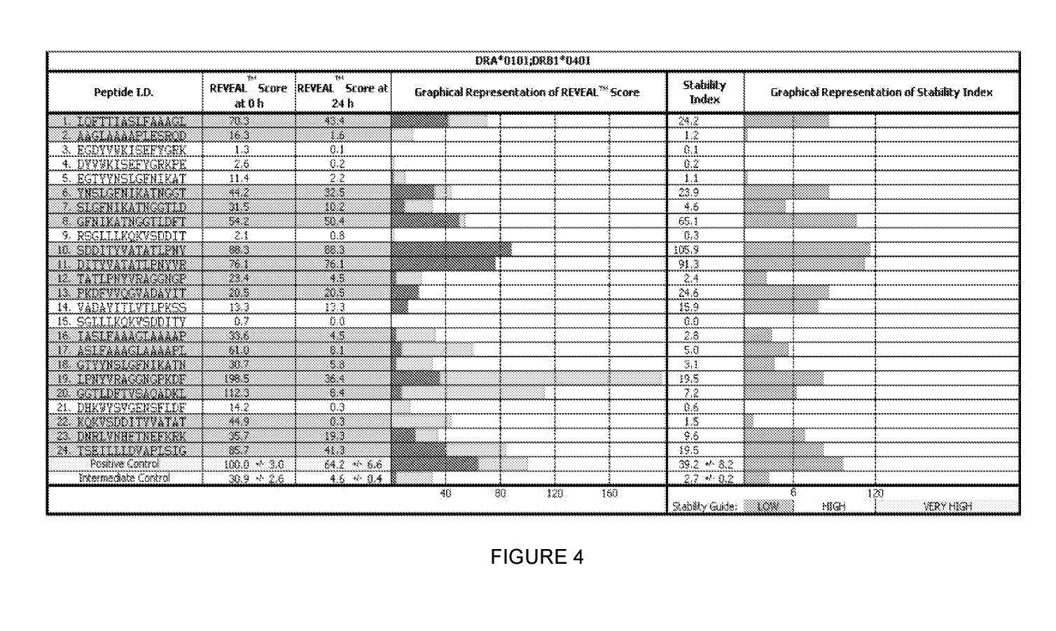

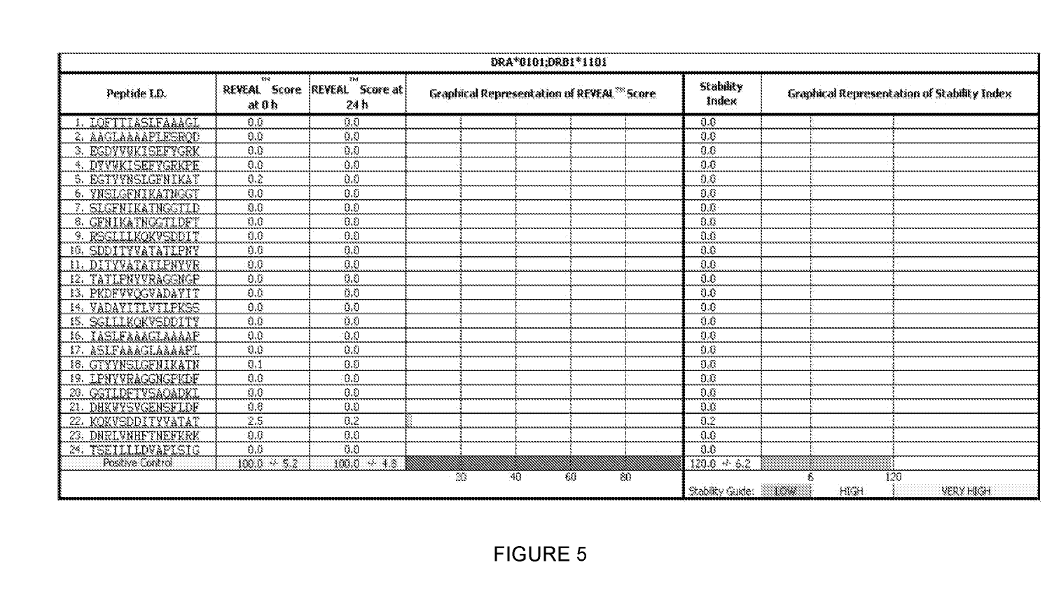

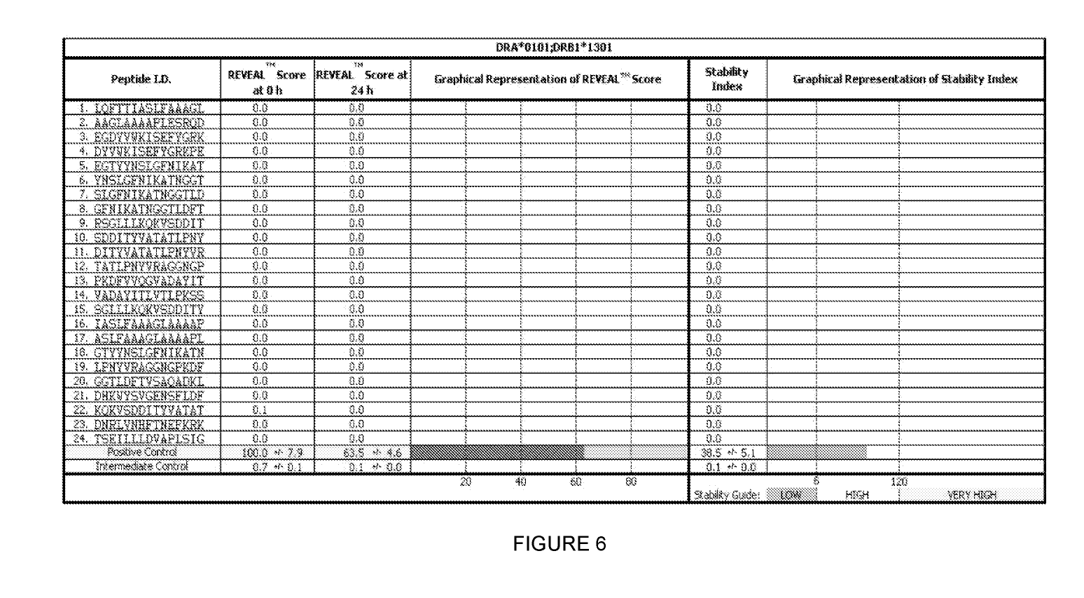

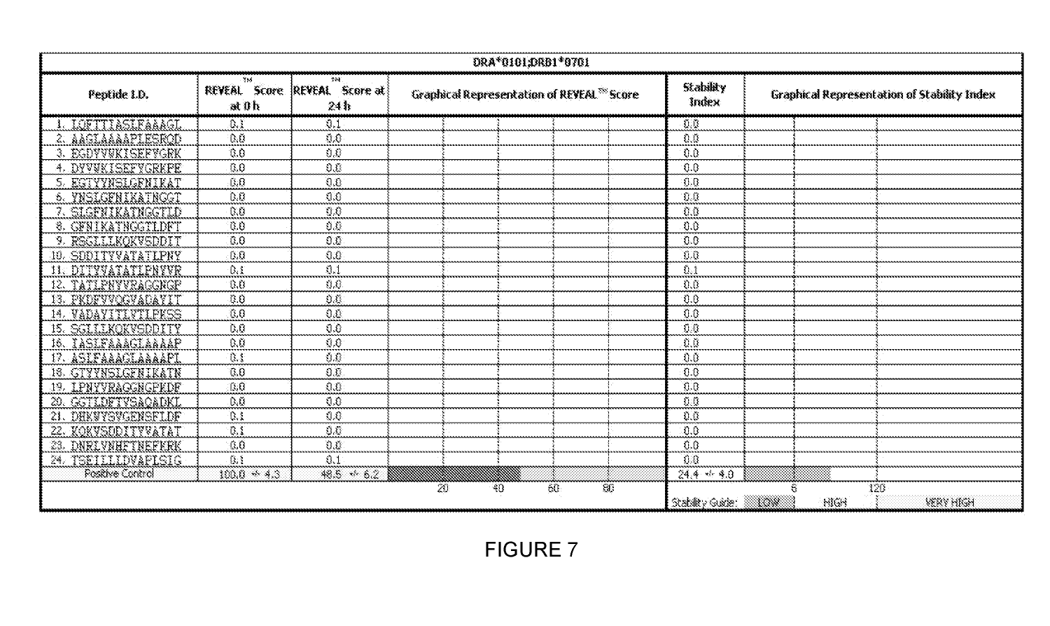

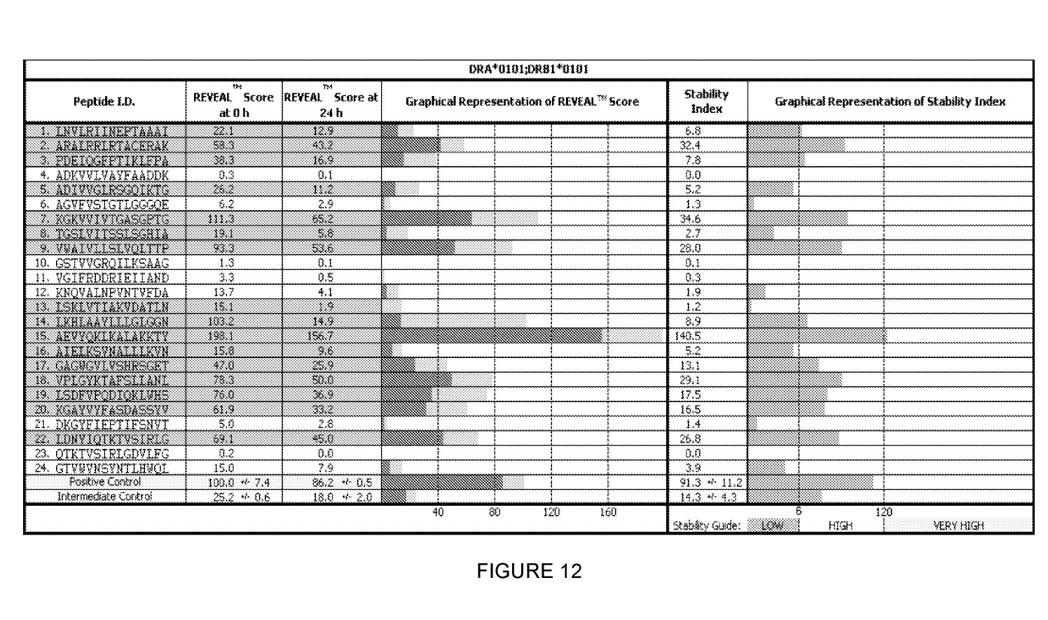

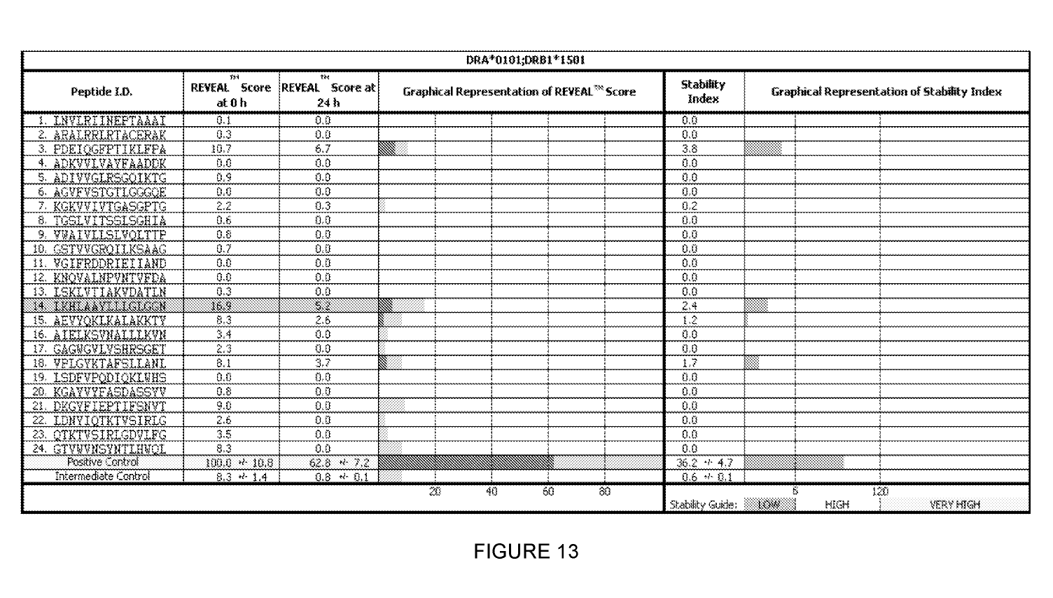

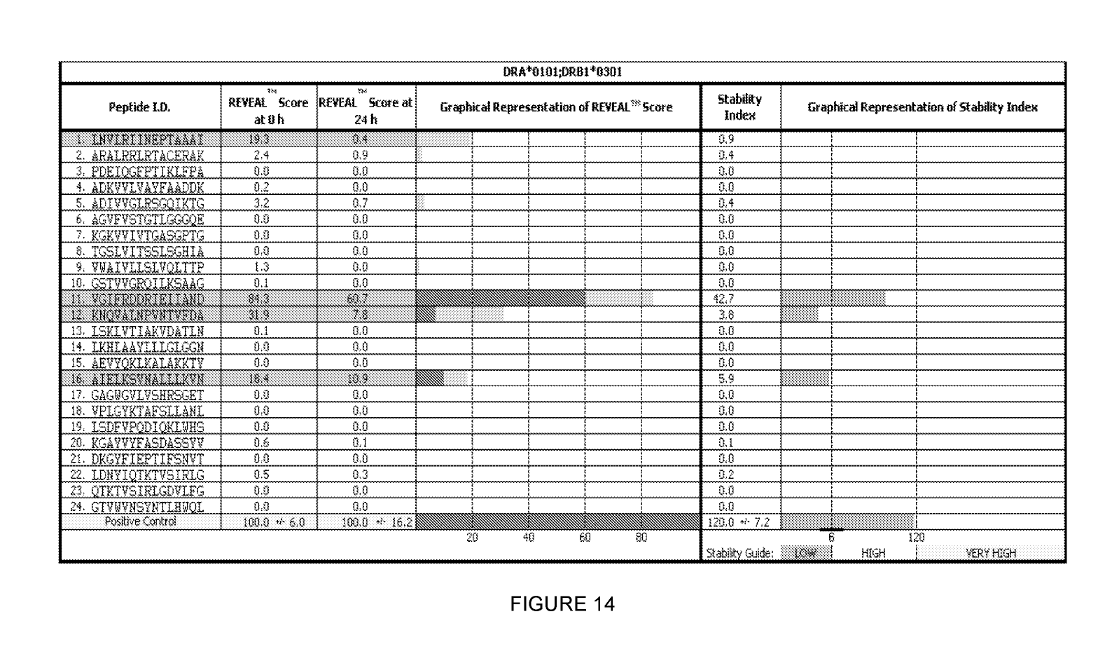

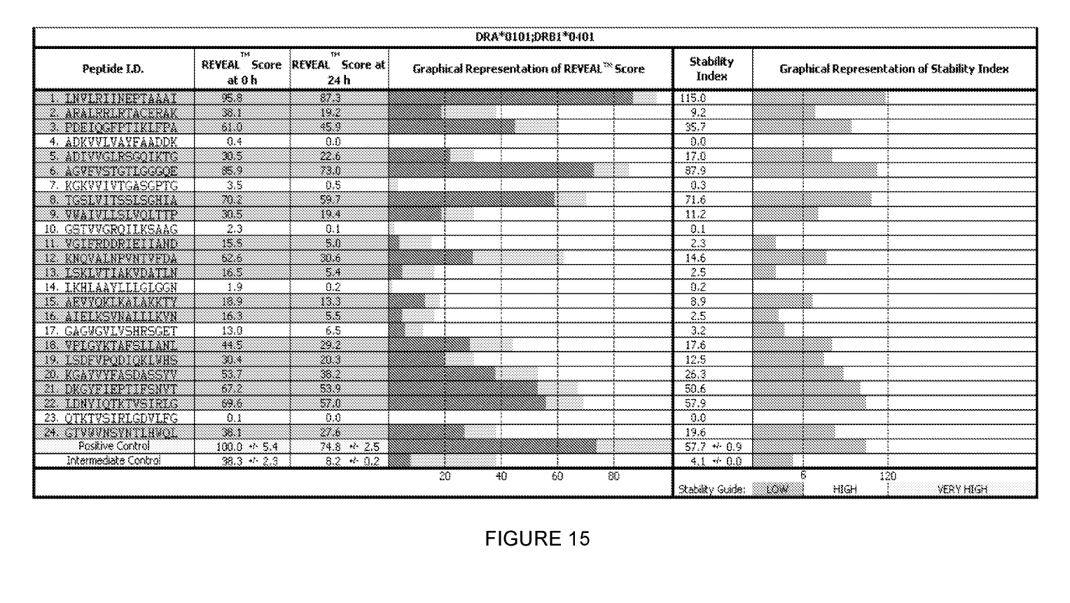

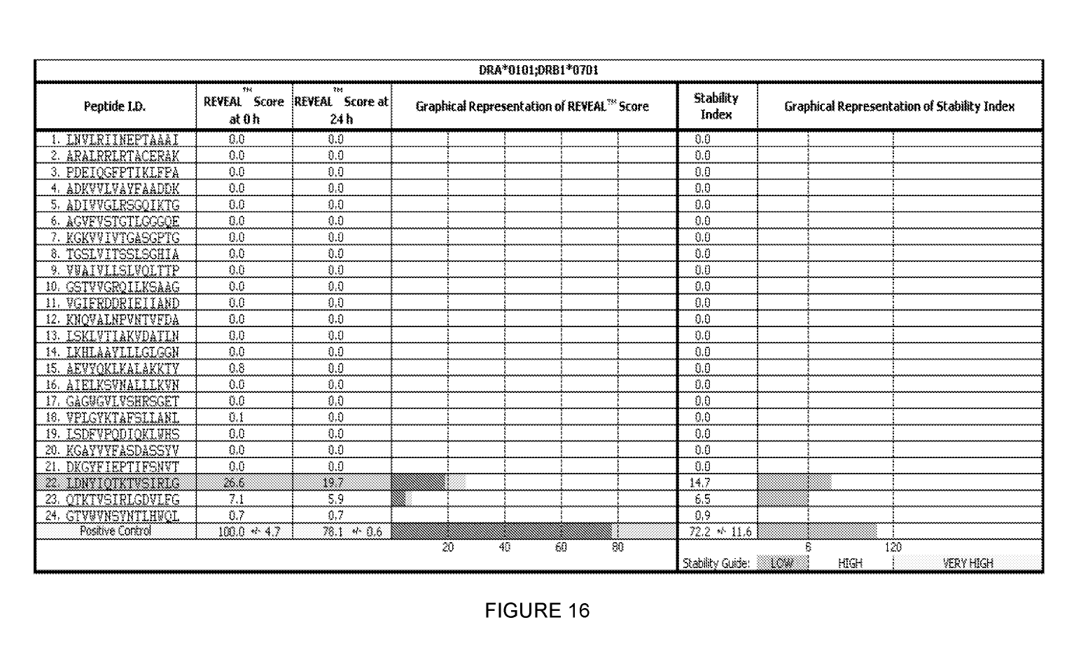

The REVEAL® binding score for each peptide-MHC complex is calculated at 0 and 24 h by comparison to the binding of the relevant positive control at 0 h and indicated numerically in FIGS. 1 to 7 and 12 to 16. These figures also show the REVEAL® score at the two time points where the score at 24 h (red cross-hatched bar) overlaps the score at 0 h (green bar) and indicates the proportion of assembled complex that has remained after the 24 h incubation.

Peptides considered to be good binders are those peptides with scores ≧15% of the positive control. These peptides are referred to as passed epitopes and are highlighted in blue. Strong T-cell epitopes tend to be identified as clear positive responses in this assay. Binding of the intermediate control is shown, where possible, so that a comparison can be made with another T-cell epitope with weaker binding characteristics.

Experimental standard error was obtained by triplicate positive and intermediate control binding experiments. The standard error for these controls is reported below and can be assumed to be representative of the degree of error that would be present for all samples.

### Module 4: Quick Check Stability Assay

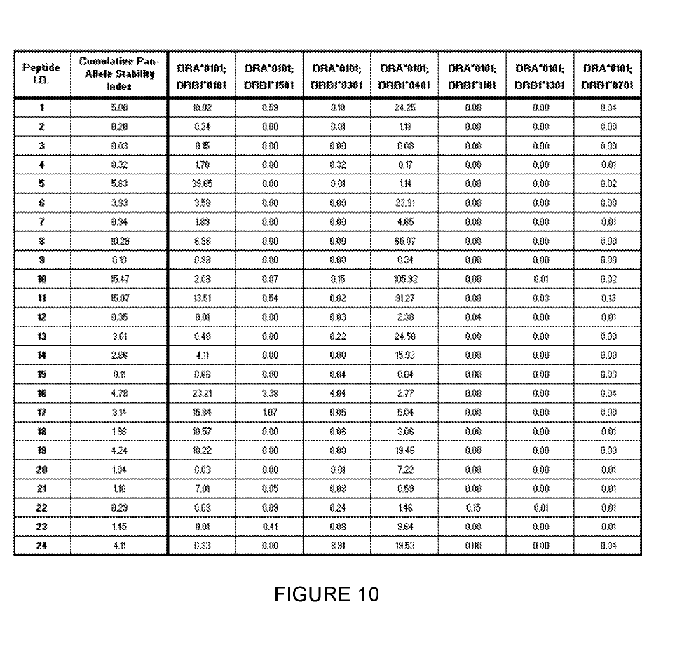

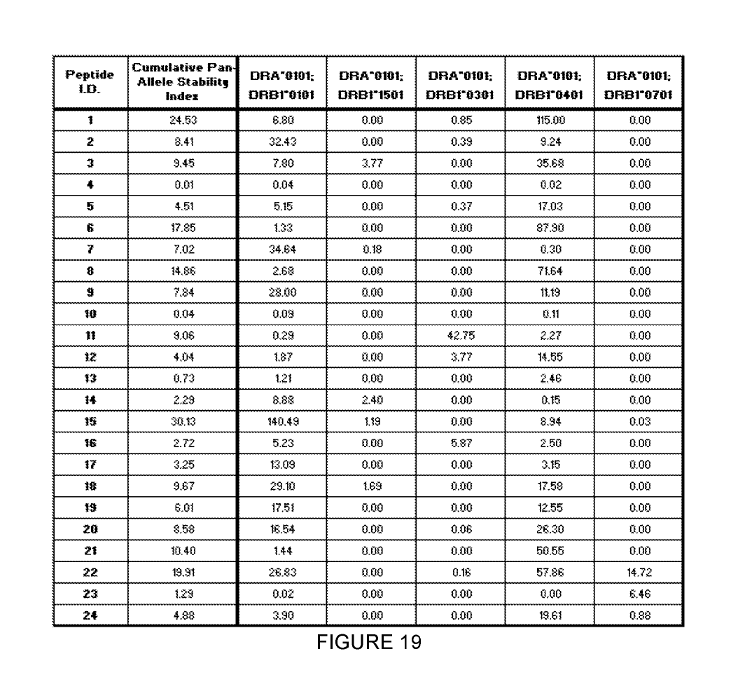

The stability of the MHC-peptide complexes from the REVEAL® assay was determined by taking measurements at 0 and 24 h during incubation at 37° C. The signals at these time points were used to estimate a half-life based on a one-phase dissociation equation. The stability index was then calculated by multiplying the half-life by the REVEAL® score from the binding assay to weight the value so that very weakly binding peptides would not be over represented. For samples with half-lives longer than 120 h, a maximum half-life of 120 h was used in the stability index calculation since a measurement period of 24 h is not long enough to estimate values above this level accurately. Further, the stability index was divided by 100 to bring the scale of values back into a more easily interpretable range. The stability indices are presented numerically in FIGS. 10 and 19 where a higher stability index is indicative of a more stable MHC-peptide complex and a better epitope. The graphical representation of stability index in these figures is presented on a logarithmic scale where the two gradations represent the three distinct levels of stability:
Low stability (red): the range in which a sample with a REVEAL® score of 100 would be stable for 6 h
High stability (yellow): the range in which a sample with a REVEAL® score of 100 would be stable for between 6 and 120 h
Very high stability (green): the range in which a sample with a REVEAL® score of 100 would be stable for 120 h

The stability indices of the test peptides was compared to those of two known T-cell epitopes from the binding assay (the positive and intermediate controls). Experimental standard error was obtained by analyzing triplicate controls and can be assumed to be representative of the degree of error that would be present for all samples.

### Pan-Allele Binding Analysis Based on REVEAL™ Score

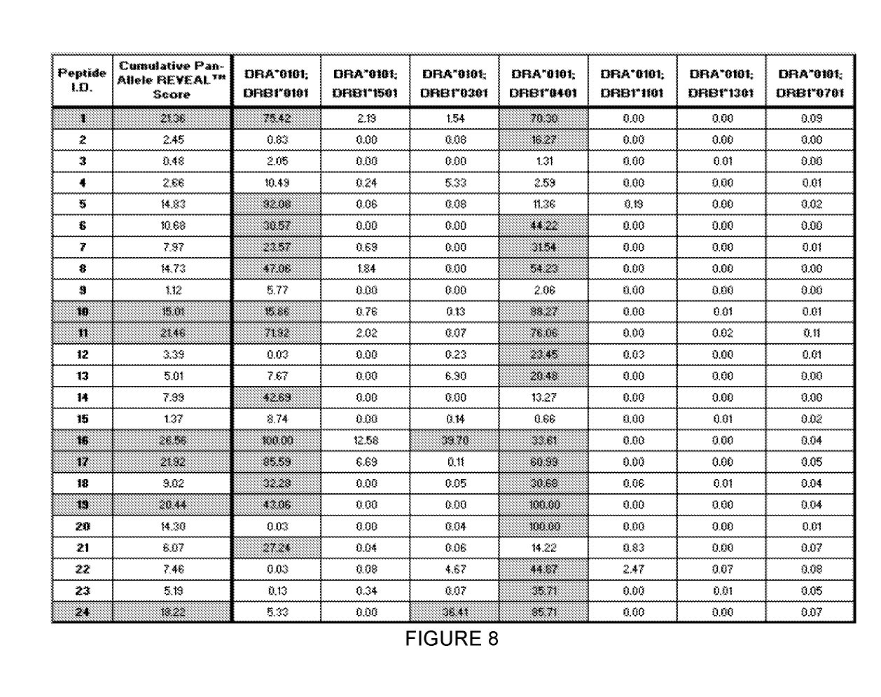

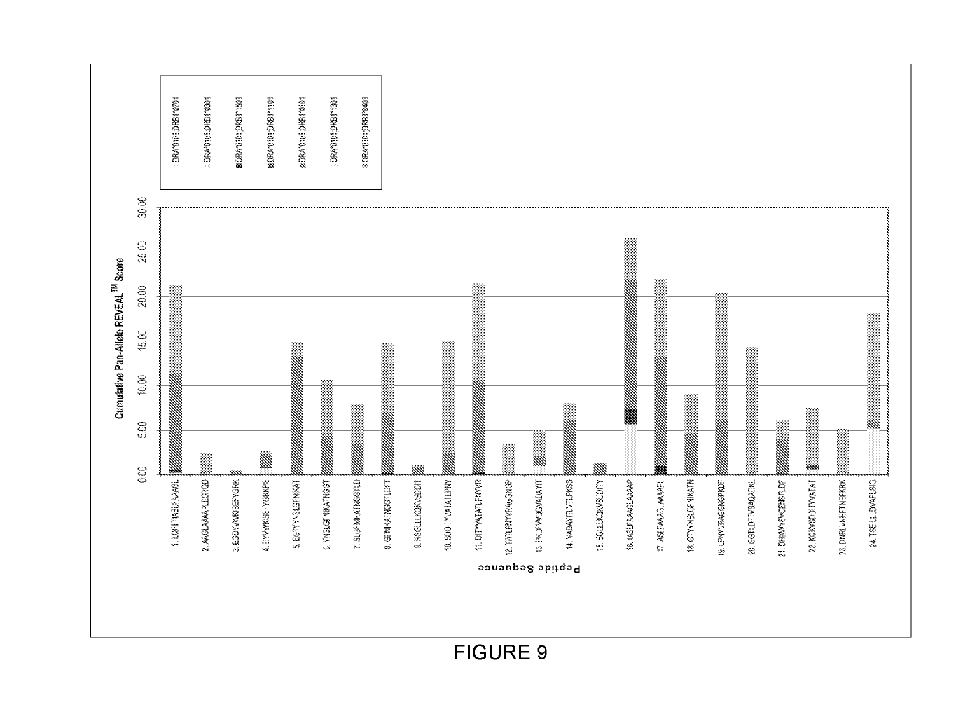

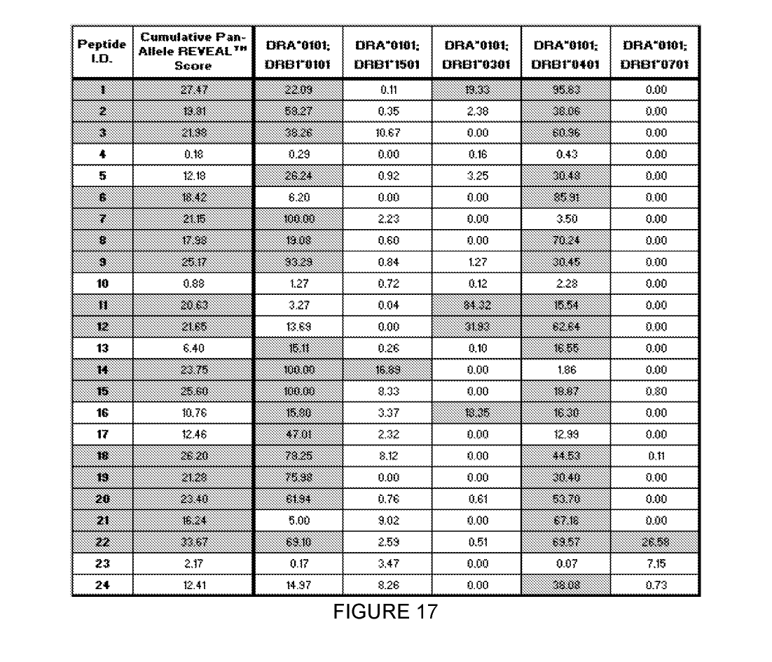

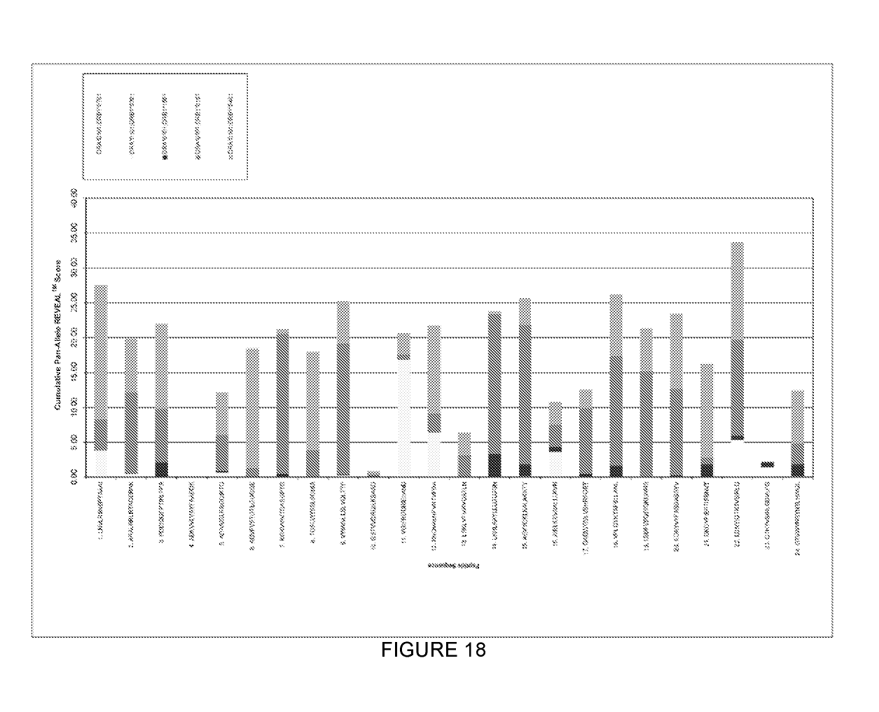

The Pan-allele binding analysis was carried out to show the cumulative binding data for each peptide across the multiple alleles studied in the REVEAL® binding assay. Those peptides whose REVEAL® scores are >100% of the positive control were standardized to a fixed value of 100% to prevent individual alleles from being over-represented in this comparison due to exceptionally high levels of binding. The standardized cumulative total of the REVEAL® scores for each peptide across all the alleles is then determined by the sum of each allele's standardized REVEAL® scores divided by the number of alleles in question. Peptides with standardized cumulative REVEAL® binding assay scores are highlighted in blue in the tabulated data (FIGS. 8 and 17) and can be referred to as pan-allele binding epitopes. The standardized cumulative REVEAL™ scores are also presented graphically in FIGS. 9 and 18, where the total bar height is made up of the individual contributions from each allele.

### Pan-Allele Stability Index Analysis

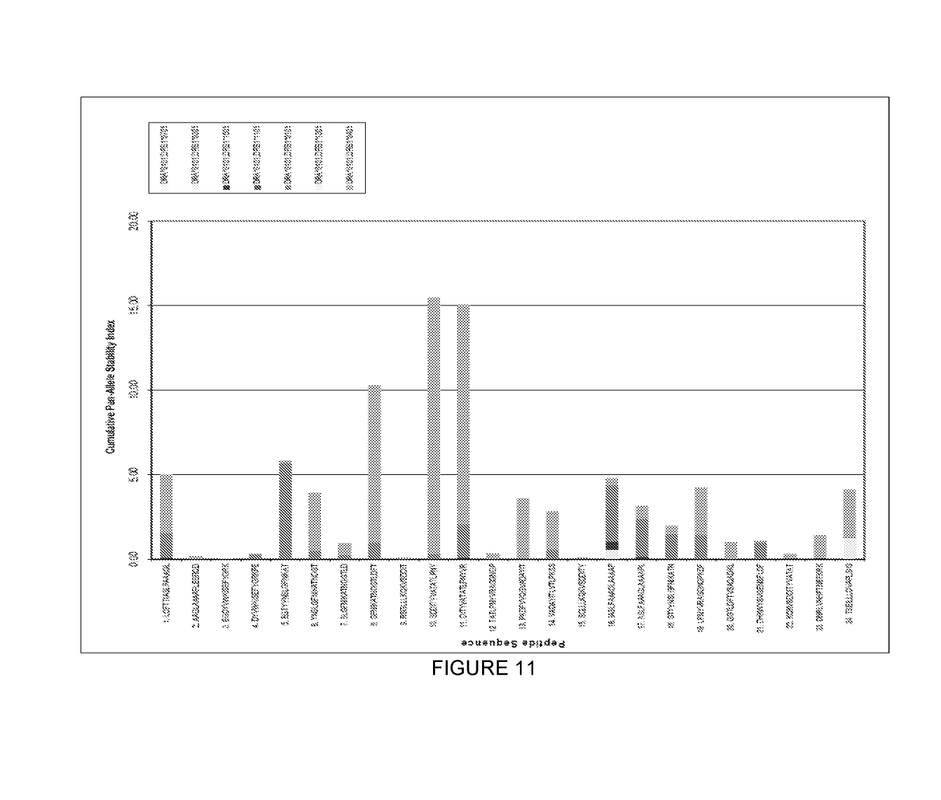

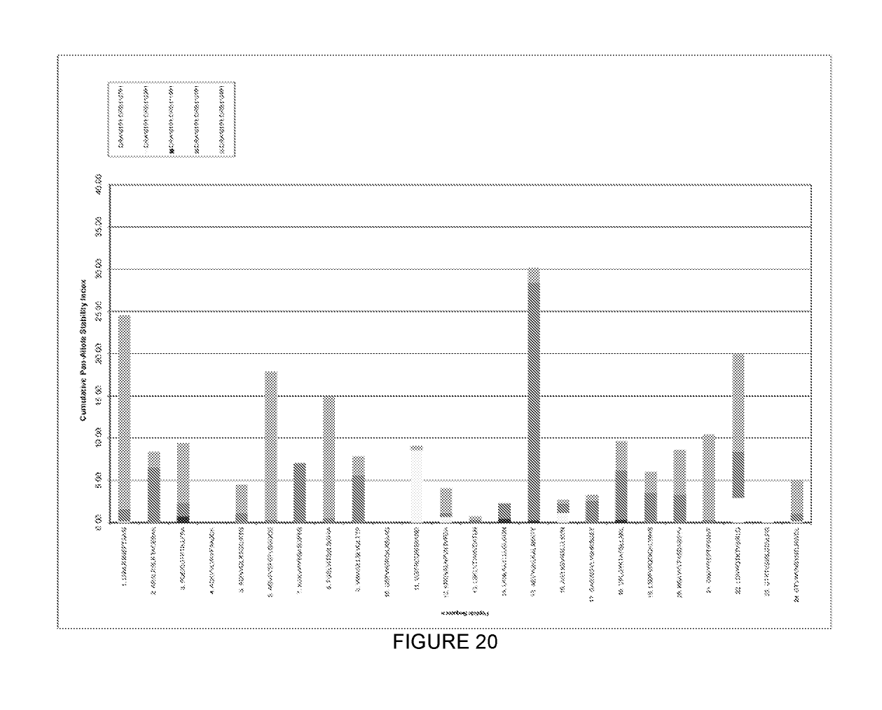

The Pan-Allele stability analysis was carried out to show the cumulative stability data for each peptide across the multiple alleles studied in the rate assays. The cumulative total of the stability index for each peptide across all the alleles is then determined by the sum of each allele's stability index divided by the number of alleles with which that peptide was tested. Each peptide's cumulative stability index is shown in the tabulated data (FIGS. 10 and 19). The standardized cumulative stability indices are also presented graphically in FIGS. 11 and 20 as the total bar height made up of the individual contributions from each allele. The positive and intermediate controls are left out of this analysis because different sets of controls are used for each allele.

### Example 4

### T-Cell Epitope Validation Assay

### Assay Description and Use

A T-cell epitope validation assay is provided to test the ability of a peptide or peptide derived from antigen presenting cell (APC) processed whole protein to be presented via a MHC molecule on an APC to form a significant affinity binding reaction with a T-cell receptor present on CD-4⁺ T-lymphocytes (along with other co-stimulatory/binding molecules). This binding reaction results in CD-4+ T-cell activation which can be measured in a number of ways.

One measure of response for activation is proliferation of the lymphocytes detected by new DNA synthesis. Background proliferation can vary considerably and therefore results are expressed as a stimulation index (SI) calculated as proliferation of cultures plus antigen (peptide/protein) divided by proliferation of cultures without antigen (peptide/protein). An SI above 2 or 3 is indicative of induced proliferation and that a T-cell epitope(s) is present.

A second method to measure activation is to measure gene expression via production of specific proteins such as cytokines. Often a single cytokine such as INF-γ or IL-4 can be used as a marker for activation. The enzyme-linked immunospot (ELISPOT) assay can provide sensitive detection of cytokine synthesis by individual cells of activated clonal populations. In this assay the number of peptide activated cells is compared to the number of background cells producing cytokine with no peptide addition. Results are reported in spot forming cells/10⁶ PMBCs.

These assays can be used to 1) define T-cell epitopes, 2) determine “exposure” of a population to antigens, 3) diagnose recognized diseases and conditions, and 4) determine the immune response to epitopes.

### Source of Cells

The immunological status of donor cells is an important variable in the assay. Typically cells from donors who are known to be sensitive/primed to the antigen (epitope) give the greatest responses in the assay. In studies investigating epitopes from allergens, donors who are classified as sensitive or allergic to the corresponding protein on the basis of skin tests or serum antibody levels will show significant responses to epitopes in the cellular assay (17). Donors who test negative for allergy to particular allergens will usually be unreactive in the stimulation assay with an SI less than 2. This type of negative control can be used to set a lower threshold for acceptance of proliferation or number of cytokine producing cells when using known sensitized donors to define an epitope. Pooled cells from many donors can be used for assay development to conserve patient samples.

Another variable is the MHC Class II (DR, DQ, and DP) type present on the donor antigen presenting cells. Epitopes in the form of peptides are MHC restricted and must have affinity for the particular Class II molecules for presentation to CD4+ lymphocytes. Donor cells may therefore be typed for Class II DR, DQ and DP. The ability of test peptides selected by ProPred and/or ProImmune REVEAL® analysis on the basis that they are Class II DR epitopes can then be compared against their ability to stimulate a response in the T-cell validation assay. This information can be used to validate the accuracy of the ProPred prediction and the results of ProImmune REVEAL® assay(s).

### Donor Cell Preparation and Assay

Cells for the assay can be prepared from anti-coagulated whole blood using density gradients. The fraction known as peripheral blood mononuclear cells (PBMC) is harvested, and consists of a variety of cell types including monocytes and different lymphocyte classes. Cells are grown from 2-7 days with and without the peptide/protein being tested. Positive proliferation controls may be used, typically using mitogens such as phytohemagglutinin (PHA) or antigens such as tetanus toxoid (TET).

After the incubation phase a proliferation detection method is used, such as [³H] thymidine incorporation for approx 6 hrs and then counts are measured or cytokine production from individual cells is detected using ELISPOT. This system is sufficient for stimulation of “sensitized/primed” cells. The T-cell epitope assay may also use an “unprimed/unsensitized” population of cells. To map epitopes in such cases (including antigens not normally in the patients environment), the assay can be modified to use specific isolated cell populations such as adherent monocytes (dendritic cells) and purified CD4+ lymphocytes (18). Similarly, cloned T cells and purified APC populations can be used to increase the sensitivity of detection.

PBMC fractions may be prepared as follows:
(i) Blood samples are withdrawn from patients with fungal infection or allergy, e.g. SAFS, and optionally from patients without fungal infection or allergy (to act as control samples). The allergy status of patients may be determined for example by skin prick tests with allergen extracts.
(ii) Whole anticoagulated blood is separated on a Ficoll gradient and the PBMC layer is withdrawn and frozen in HSA/DMSO and stored in liquid Nitrogen.
(iii) Cells are defrosted as required, counted, plated at 10⁶ cells/ml, 200 μl/well 2.0×10⁵ cells/well, 96 well round bottom plate, 37° C., 5% CO₂. Culture media: AIM V (BD), contains 0.25% HAS, 50 μg/ml Streptomycin, 10 μg/ml Gentamycin and L-glutamine.

For positive controls: TET stock is diluted and applied 24 hours after plating out cells at a concentration 0.5 μg/ml.

Control populations may be analysed by including PBMC from a group of patients who have allergy but do not have evidence of fungal sensitization and a group of “normal” volunteers without allergies or other diseases. Such experiments serve to define the T-cell reactivity of the populations to fungal epitopes for comparison. The “normal” volunteers also provide a negative control for the assay.

### Form of Epitopes

Stimulation of T-cells has been reported using whole proteins and defined peptides of varying lengths. It is assumed that whole proteins are internally processed and peptides are presented by APC's while epitopes added as peptides bind directly to empty (and possibly occupied) MHC molecules on the cell surface. Class II MHC molecule binding pockets will contact and hold a 9 amino acid epitope core. The MHC Class II pocket is open at both ends so varying lengths of peptide can over hang. Peptides up to a total length of 20-30 amino acids have been found to bind Class II molecules. Stimulation experiments typically use chemically synthesized peptides between 10-25 amino acids and obtain positive results. One strategy is to produce a “PepSet” or “Epitope Scan” consisting of a set of peptides between 15-25 amino acids in length which are offset by 3-10 amino acids and span the entire length of a protein.

### Example 5

### Cytokine Analysis

Cytokine analysis can greatly enhance the information content of the stimulation assay. Wth the discovery of the polarization of T-cells into distinct cytokine producing populations (Th0, Th1 and Th2) the assay has been used to study the effects of epitopes on T-cell differentiation (19).

Cell culture supernatants can be assayed for cytokines using a number of standard assay formats. The use of FACS analysis to evaluate the cells and molecules in the stimulation assay increases the flexibility of analytical analysis. A FACS method can differentiate cell types, measure proliferation, and measure intracellular cytokine levels (20). An ELISPOT assay allows analysis of cells at the single cell level for multiple cytokine production (28)

### Proliferation and Cytokine Detection Methods

### 1. FACS Based FastImmune™ Anti-BrdU Proliferation Assay:

Bromodeoxyuridine (BrdU) is a uridine derivative that can be incorporated specifically into DNA in place of thymidine. Anti-BrdU identifies BrdU (but not thymidine) in single-stranded DNA, free BrdU or BrdU coupled to a protein carrier. DNase-I cleaves each strand of DNA at random and permits the binding of Anti-BrdU antibody to its antigen (BrdU).

Cells can be pulse-labelled with BrdU, and those cells that are synthesising DNA will incorporate BrdU into the DNA. Anti-BrdU can then be used to identify cells that undergo DNA synthesis during exposure to BrdU.

Cells are bromodeoxyuridine (BrdU) labelled and stained at 7 days post application of antigen. BrdU is added to wells to give a final concentration of 0.01 mM, 5 hours before staining.

Cells are fixed, permeabilized and stained. Staining antibody BD anti-BrdU-FITC, supplied as a solution already containing DNase (Becton Dickinson Fastlmmune Catalog Number 340649). Solutions used are PBS 1% BSA, BD FACS Perm 2 solution and PBS 1% Paraformaldehyde.

Cells fixed and stained as above are stored at 4° C. overnight before running on a FACS machine the following day.

### 2. FACS Based Fastimmune™ CD4 Intracellular Cytokine Detection

FACS based Fastimmune™ intracellular cytokine detection kits (Becton Dickinson Catalog Nos. 337185, 337187, 337189) are used to detect CD4+, CD69+ and Interferon γ (INF-γ) status of the T-cell population.

### 3. Th1/Th2 Status Determination

A Cytometric Bead Array (CBA) Human Th1/Th2 Cytokine Kit (Becton Dickinson Catalog Number 550749) is used to detect the Th1/Th2 status of the T-cell population.

This assay kit enables one to detect production of IL-2, IL-4, IL-5, IL-10, THF, and IFN-γ in liquid samples and determine the Th1 or Th2 status of a T cell population.

### 4. ELISPOT Assay for Cytokine Detection

The ELISPOT assay uses two high affinity cytokine specific antibodies, directed against two different epitopes on the same cytokine molecule (28).

96 well PVDF membrane ELISPOT plates are coated with capture antibody in coating buffer and incubated O/N at 4° C. Plates are washed and then blocked with complete RPMI-1640 media at RT for 1 h. Cells, peptides, allergens and controls in RPMI-1640 media will be aliquoted to wells and incubated at 37° C., 5% CO₂ in a humidified incubator for 24-48 h. Cells and medium are decanted and the plates are washed 3× with wash buffer. Biotinylated detection antibody in assay diluent is added to the plates are incubated at RT for 2 h. Plates are then washed 4× and aviden-horseradish peroxidase reagent is added and the plates are incubated at RT for 45 min. Plates are washed 3× with wash buffer and 2× with PBS. AEC substrate is added for 10-60 min at RT and spot development is followed and the reaction is stopped with diH₂O. The plates are air dried and the spots are counted with an automated ELISPOT plate reader.

### Example 6

Twenty (20) patients sensitised to Alternaria alternata and exhibiting symptoms of allergic rhino sinusitis will be identified. Wth the appropriate consent, a blood sample will be collected from each patient. Patients will be evaluated at the start of the trial by monitoring blood levels of antibodies, respiratory testing, HLA typing and skin-prick tests.

Peptides identified from ProImmune REVEAL® analysis as promiscuous and strong HLA DR allele binders will be validated in vitro against patient blood samples using an IL-4 ELISPOT assay.

In particular, 10 patients will not receive Alternaria extract and 10 patients will be given Alternaria extract intranasally. After two weeks, blood samples will be taken and ELISPOT IL-4 cytokine assays performed. All samples will be challenged in vitro with the candidate peptide(s) and the effect of the peptide(s) on IL-4 cytokine production assessed by ELISPOT assay.

### Example 7

### Measurement of IL-4 Response Following Peptide Stimulation of Blood Samples from Human Patients

Forty three (43) 15mer peptides identified from ProImmune REVEAL® analysis as promiscuous and strong HLA DRB1 allele binders were validated by in vitro analysis of patient blood and control group blood samples using an IL-4 ELISPOT assay.

The 43 peptides are predicted to be positive binders selected from the group of 48 peptides listed in FIGS. 21 and 22. The five peptides excluded from the analysis were 34.2, 36.2, L6-1 (shaded in the “15mer ALG Code” column in FIGS. 21) and 54.2 and 49.2.1 (shaded in the “15mer ALG Code” column in FIG. 22).

Patients from the Allergy Unit, Hospital Clinic, University of Barcelona (Barcelona, Spain) were selected and divided into two groups:
1. Nineteen (19) test patients sensitised to Alternaria alternata:
   All 19 test patients had allergic rhinitis and conjunctivitis and a majority of test patients had asthma.
   All 19 test patients were skin prick test positive to Alternaria extract and positive for nasal challenge with Alternaria extract.
   17 of 19 of the test patients were tested for Basophil activation by rAlt a 1 and were found to be positive.
   Test patients were positive for IgE response to Alternaria extract.
2. Fifteen (15) control patients
   All control patients were negative for skin prick test to Alternaria extract and negative for specific nasal challenge with Alternaria extract.

Response to nasal challenge with Alternaria was measured by acoustic rinometry, where challenge was considered positive if the nasal challenge with diluent was negative and the volume between 2nd and 5th cm into the nose closed >25% after the Alternaria challenge (as described in Allergen-specific nasal provocation testing: review by the rhinoconjunctivitis committee of the Spanish Society of Allergy and Clinical Immunology. Dordal M T, Lluch-Bernal M, Sánchez M C, Rondón C, Navarro A, Montoro J, Matheu V, Ibáñez M D, Fernández-Parra B, Dávila I, Conde J, Antón E, Colás C, Valero A; SEAIC Rhinoconjunctivitis Committee. J Investig Allergol Clin Immunol. 2011; 21(1):1-12).

The basophil activation test (Basotest™ Glycotope Biotechnology GmbH, Heidelberg, Germany) in response to rAlt a 1 was considered positive if the percentage of Basophil activation was greater than 20% with a stimulation index (SI: test value/background value) higher than 2.

Wth the appropriate consent, blood samples were collected from each patient for analysis at the start and during the trial.

Patients were evaluated at the start of the trial by monitoring blood levels of antibodies, respiratory testing, HLA typing and skin-prick tests. In particular, serum total IgE and specific IgE response to Alternaria and Alt a1 was measured (ImmunoCAP Phadia and Microarray ISAC Phadia).

PBMC were isolated from heparinised whole blood by centrifugation in Ficoll-Paque, washed and put into serum free freezing media containing DMSO for storage in liquid N₂. All cell-culture media is serum-free. Thawed PBMC were used at 350,000 cells per well with or without peptide. The final concentration of each peptide is 10 μg/ml per assay well either individually or as a member of a pool. Incubation is at 37° C. and 5% CO₂ for 48 hours for the IL-4 assay, after which time plates are processed for the detection of IL-4 production.

PBMC samples from test and control patients were prepared from blood samples, challenged with candidate peptide and subjected to ELISPOT assay to determine IL-4 response to peptide stimulation.

For challenge, peptides were administered as follows:
Individual peptides (soluble). Individual water soluble peptides were dissolved as 10, 5 or 2.5 mg/ml stock solutions in sterile distilled water. Before use, each peptide was diluted in water to 2 mg/ml and 10 μl of this 2 mg/ml mix was added to 990 μl of media. Then 100 μl of this peptide/media mix was added to 100 μl of media containing 350,000 PBMC in an ELISPOT anti-IL-4 coated cell culture plate well for a final peptide concentration of 10 μg/ml. Identically diluted media only was added to the cells and used for the no peptide media control wells (media only).
Individual peptides (DMSO). Individual water insoluble peptides were dissolved in 100% DMSO to a stock concentration of 50 mg/ml. Before use each peptide was diluted in water to 2 mg/ml and then 10 μl of the 2 mg/ml mix was added to 990 μl of cell-culture media. Then 100 μl of this peptide/media mix was added to 100 μl of media containing 350,000 PBMC in an ELISPOT anti-IL-4 coated cell culture plate well for a final peptide concentration of 10 μg/ml. Identically diluted media only was added to the cells and used for the no peptide media control wells (media only).
A combination preparation containing Alt a 1 soluble peptides (33.2, 34.3, 35.4, 35.5, 37.3.1, 37.4.1, L6-5.01, L10-1.01, L10-2.01, L10-3), called Alt a 1 Sol. Each peptide was diluted from water stock to 2 mg/ml in sterile distilled water. Then 10 μl of each peptide required for the combined preparation was mixed and brought up to 1 ml in media. Then 100 μl of this peptide/media mix was added to 100 μl of media containing 350,000 PBMC in an ELISPOT anti-IL-4 coated cell culture plate well for a final concentration of each peptide of 10 μg/ml. Identically diluted media only was used for the no peptide control wells (media only).
A combination preparation containing Alt a 1 DMSO peptides (32.2.1, 38.2.1, 35.2, 35.3, L6-2, L6-3, L6-4, 38.3, 37.2), called Alt a 1 DMSO. Each peptide was diluted from DMSO stock to 2 mg/ml in sterile distilled water. Then 10 μl of each peptide required for the combined preparation was mixed and brought up to 1 ml in media. Then 100 μl of this peptide/media mix is added to 100 μl of media containing 350,000 PBMC in an ELISPOT anti-IL-4 coated cell culture plate well for a final concentration of each peptide of 10 μg/ml. Identically diluted media only was used for the no peptide control wells (media only).
A combination preparation containing non-Alt a 1 soluble peptides (40.2, 41.2, 43.2, 44.2, 46.2, 47.2.1, 56.2, 57.2.1, 60.2, 62.2.1, 67.2, 71.3), called Alt a Other Sol. Each peptide was diluted from water stock to 2 mg/ml in sterile distilled water. Then 10 μl of each peptide required for the combined preparation was mixed and brought up to 1 ml in media, Then 100 μl of this peptide/media mix was added to 100 μl of media containing 350,000 PBMC in an ELISPOT anti-IL-4 coated cell culture plate well for a final concentration of each peptide at 10 μg/ml. Identically diluted media only was used for the no peptide control wells (media only).
A combination preparation containing non-Alt a 1 DMSO peptides (39.2, 42.2, 45.2, 48.2.1, 50.2, 51.2.1, 61.2.1, 66.3.1, 68.2.1, 70.2, 71.2, 72.2), called Alt a Other DMSO. Each peptide was diluted from DMSO stock to 2 mg/ml in sterile distilled water. Then 10 ul of each peptide required for the combined preparation was mixed and brought up to 1 ml in media, Then 100 μl of this peptide/media mix was added to 100 μl of media containing 350,000 PBMC in an ELISPOT anti-IL-4 coated cell culture plate well for a final concentration of each peptide at 10 μg/ml. Identically diluted media only was used for the no peptide control wells (media only).

### Results

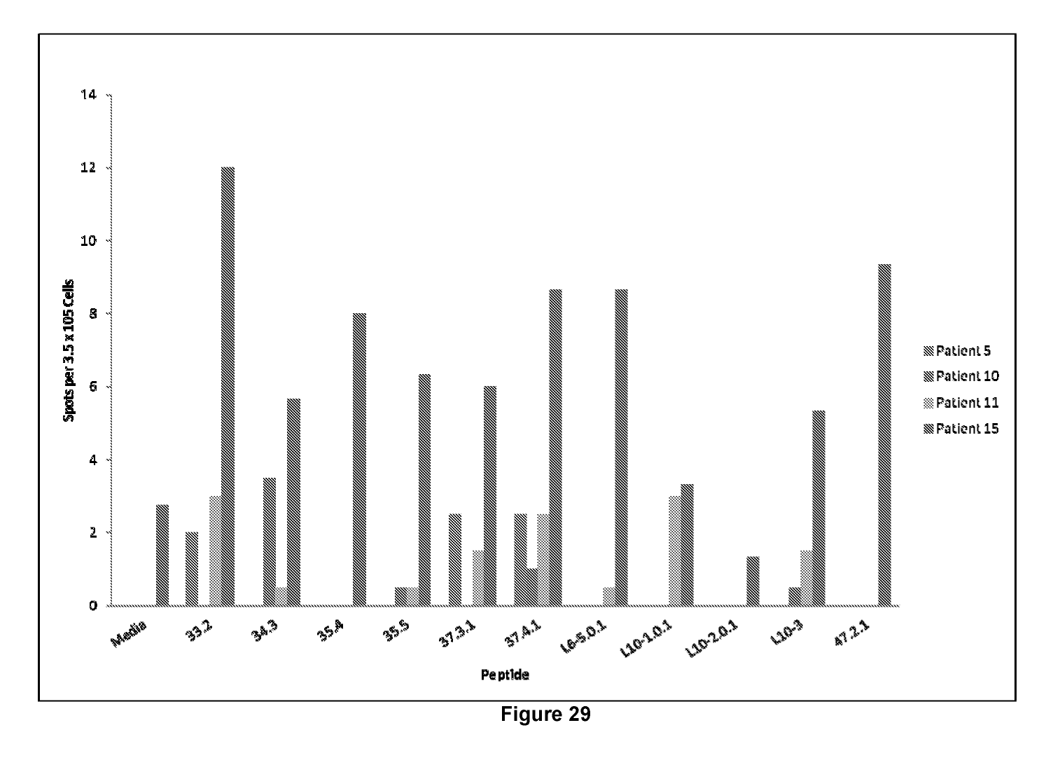

Results are summarised in FIGS. 29 to 32.

A large subset of the peptides tested stimulated IL-4 counts in test patient PBMC over no peptide background (media only) and control patient PBMC. Most of the positive peptides caused stimulation of multiple patients.

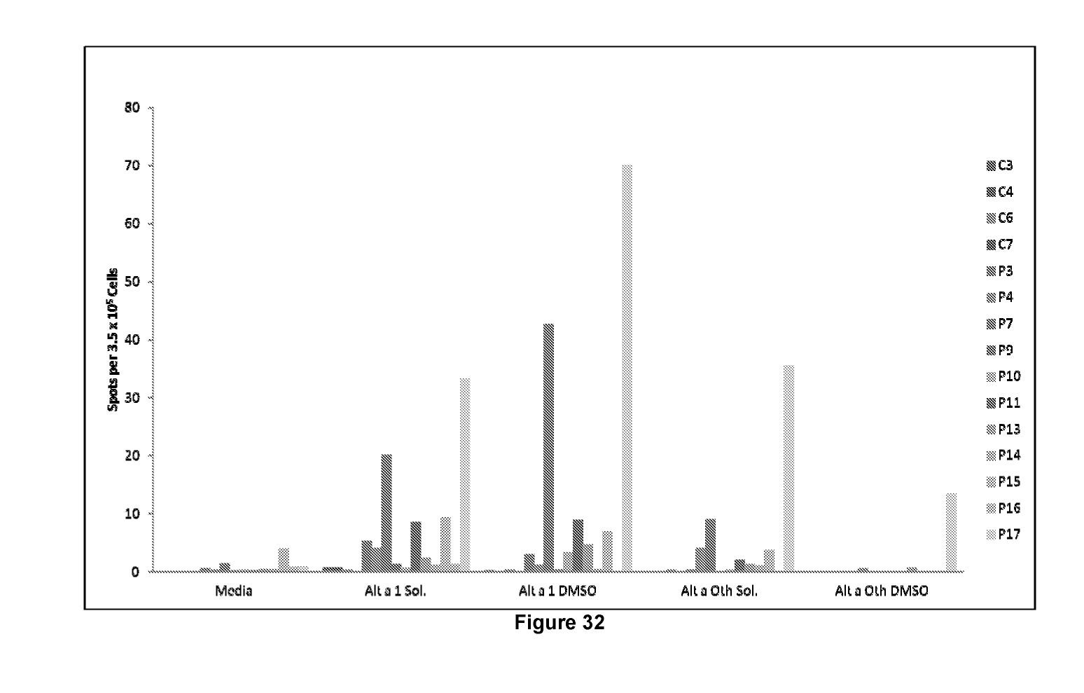

For the combined preparations, data was obtained from all of the control patients and test patients listed in the legend to FIG. 32 for Alt a 1 Sol, Alt a 1 DMSO, and Alt a 1 Other Sol. FIG. 32 indicates that the control patients were negative for IL-4 stimulation compared to media. For the Alt a Other DMSO preparation, only 5 patients were tested: P3, P4, P7, P13, P17.

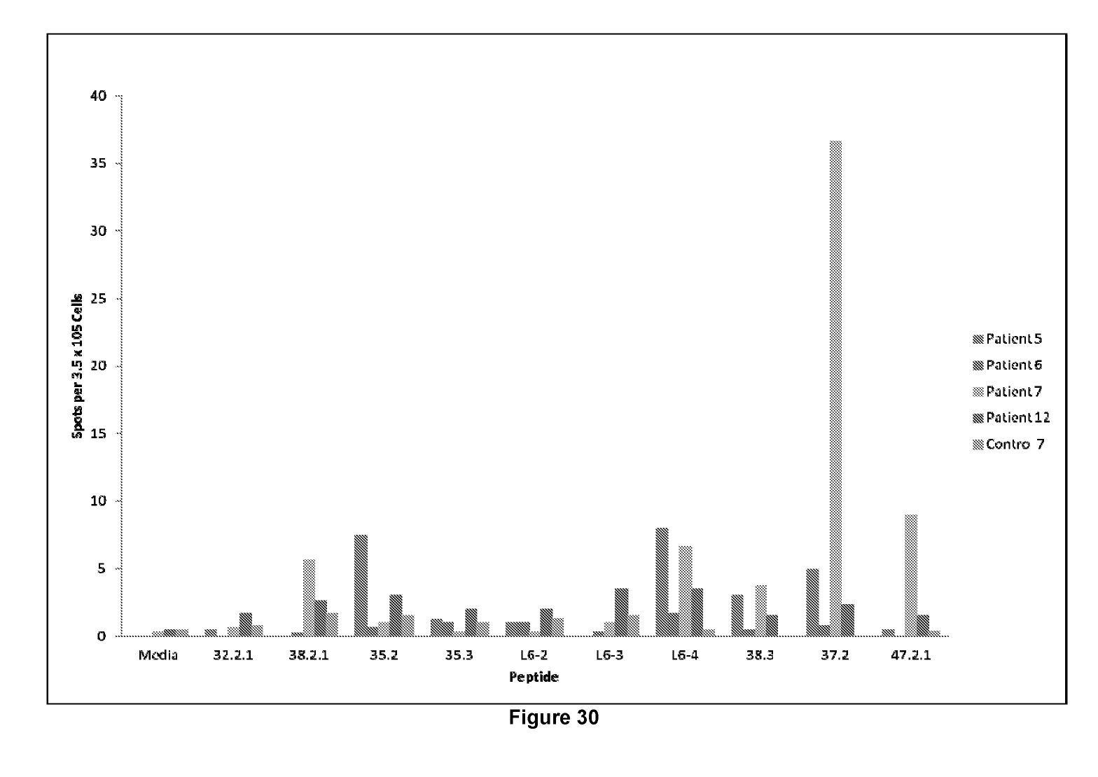

FIGS. 29 and 30 show results of IL-4 stimulation by individual; Alt a 1 peptides as well as the Alt a 8 peptide 47.2.1.

FIGS. 29 and 30 show positive stimulation of IL-4 induction in response to a range of individual Alt a 1 peptides compared to media only control.

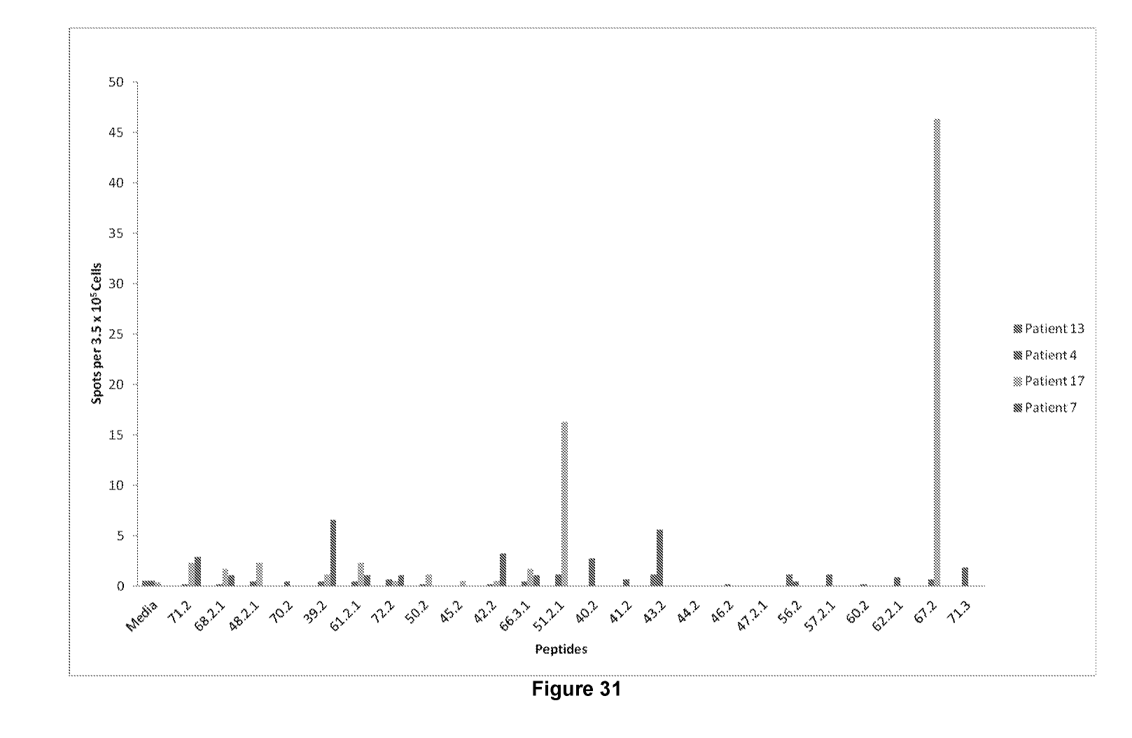

FIG. 31 shows positive stimulation of IL-4 induction in response to a range of individual Alt a 3, Alt a 4, Alt a 6, Alt a 7, Alt a 8, and Alt a 10 peptides compared to media only control.

FIG. 32 shows positive stimulation of IL-4 induction of combined peptide preparations compared to media only control and to control patients.

### Discussion

Induction of IL-4 cytokine in response to peptide stimulation is indicative of the peptide containing a T-cell epitope, and the results obtained in this study confirm the presence of T-cell epitopes in selected peptides tested.

Induction of an IL-4 response is indicative of an allergic response to a peptide independent of a diagnosis. Where a Th2 (IL-4+) population of CD4+ T-cells responds to selected peptides, those peptides can be used to induce tolerance and downregulate the Th2 allergic response to those allergens from where the peptides are derived.

Thus, the data obtained from this study validates selected peptides as useful in the prevention and/or treatment of allergic disease mediated by the corresponding protein allergen.

### REFERENCES

1. Denning et al. Eur. Respir. J. 2006. 27: 615-626.
2. Bowyer et al. Med. Mycol. 2006. 44 S23-S28.
3. Maguire et al. Clin. Immunol. 1999. 93: 222-231.
4. Haselden et al. J. Exp. Med. 1999. 189:1885-1894.
5. Bowyer and Denning. Med. Mycol. 2007. 45:17-26.
6. Schneider et al. Clin. Exp. Allergy. 2006. 36:1513-1524.
7. Shankar et al. Mol. Immunol. 2006. 43:1927-1932.
8. Singh and Raghava. Bioinformatics 2001. 17:1236-1237.
9. Bian and Hammer. Methods 2004. 34:468-475.
10. Mustafa and Shaban. Tuberculosis 2006. 86:115-124.
11. Janssen et al. J. Immunol. 2000. 164:580-588.
12. Verhoef et al. PLoS Med. 2005. 2:253-261.
13. Zuleger et al. Vaccine 2005. 23:3181-3186.
14. Caccamo et al. Clin. Exp. Immunol. 2003. 133:260-266.
15. Doytchinova and Flower. J. Immunol. 2005. 174:7085-7095.
16. Sturniolo et al. Nat. Biotechnol. 1999. 17:555-561.
17. Rawle et al. J. Immunol. 1984. 133:195-201.
18. Stickler et al. J. Immunother. 2000. 23:654-660.
19. Rogers and Croft. J. Immunol. 1999. 163:1205-1213.
20. Mehta and Maino. J. Immunol. Methods 1997. 208:49-59.
21. Klitz et al. Tissue Antigens 2003. 62:296-307.
22. Rudin et al. Allergy 2001. 56:1042-1048.
23. Marsland et al. Proc. Natl. Acad. Sci. U.S.A. 2004. 101:6116-6121.
24. Kheradmand et al. J. Immunol. 2002. 169:5904-5911.
25. De Palma et al, Allergy, 2000. 55: Suppl 61:56-59.
26. Schwabe N & A Turner, Genetic Engineering & Biotechnology News, 2008. Vol 28, No. 4
27. Larche, Chest, 2007. 132: 1007-1014
28. Smith et al., Clin. Diagn. Lab Immunol. 2001.8: 871-879
29. Kinnunen et al. J. Allergy Clin. Immunol. 2007. 119:965-972.
30. Godkin et al. J. Immunol. 2001. 166:6720-6727

1. A peptide having an amino acid length of from 8 to 50 amino acids that is selected from (a) a peptide consisting of or comprising the amino acid sequence of one of SEQ ID NOs:10, 196-210, or one of SEQ ID NOs:11, 211-233, or one of SEQ ID NOs:1-9, 12-195, 234-913, and (b) a peptide having a contiguous amino acid sequence having at least 70% sequence identity to the amino acid sequence of one of SEQ ID NOs:10, 196-210, or one of SEQ ID NOs:11, 211-233, or one of SEQ ID NOs:1-9, 12-195, 234-913.

2. (canceled)

3. The peptide according to claim 1 wherein the sequence identity is at least 80%, 85%, 90% or 95%.

4. The peptide according to claim 1 having a maximum length of 30 amino acids and a minimum length of 9 amino acids.

5. The peptide according to claim 1 having a maximum length of 20 amino acids and a minimum length of 11 amino acids.

6. The peptide according to claim 1 which is capable of stimulating an immune response.

7. A peptide comprising the amino acid sequence of one of SEQ ID NOs:10, 11, 1-9, 12-58, or a peptide having a contiguous amino acid sequence having at least 80% sequence identity to the amino acid sequence of one of SEQ ID NOs:10, 11, 1-9, 12-58, wherein the peptide has an amino acid length of from 8 to 50 amino acids.

8. (canceled)

9. A peptide consisting of the amino acid sequence set forth in one of:
(i) SEQ ID NO: 10 or SEQ ID NOs: 196-210;
(ii) SEQ ID NO: 11 or SEQ ID NOs: 211-233;
(iii) SEQ ID NO: 1 or SEQ ID NOs: 59-80;
(iv) SEQ ID NO: 2 or SEQ ID NOs: 81-92;
(v) SEQ ID NO: 3 or SEQ ID NOs: 93-105;
(vi) SEQ ID NO: 4 or SEQ ID NOs: 106-120;
(vii) SEQ ID NO: 5 or SEQ ID NOs: 121-135;
(viii) SEQ ID NO: 6 or SEQ ID NOs: 136-150;
(ix) SEQ ID NO: 7 or SEQ ID NOs: 151-165;
(x) SEQ ID NO: 8 or SEQ ID NOs: 166-180;
(xi) SEQ ID NO: 9 or SEQ ID NOs: 181-195;
(xii) SEQ ID NO: 12 or SEQ ID NOs: 234-248;
(xiii) SEQ ID NO: 13 or SEQ ID NOs: 249-263;
(xiv) SEQ ID NO: 14 or SEQ ID NOs: 264-278;
(xv) SEQ ID NO: 15 or SEQ ID NOs: 279-293;
(xvi) SEQ ID NO: 16 or SEQ ID NOs: 294-307;
(xvii) SEQ ID NO: 17 or SEQ ID NOs: 308-317;
(xviii) SEQ ID NO: 18 or SEQ ID NOs: 318-332;
(xix) SEQ ID NO: 19 or SEQ ID NOs: 333-347;
(xx) SEQ ID NO: 20 or SEQ ID NOs: 348-362;
(xxi) SEQ ID NO: 21 or SEQ ID NOs: 363-377;
(xxii) SEQ ID NO: 22 or SEQ ID NOs: 378-392;
(xxiii) SEQ ID NO: 23 or SEQ ID NOs: 393-407;
(xxiv) SEQ ID NO: 24 or SEQ ID NOs: 408-422;
(xxv) SEQ ID NO: 25 or SEQ ID NOs: 423-437;
(xxvi) SEQ ID NO: 26 or SEQ ID NOs: 438-452;
(xxvii) SEQ ID NO: 27 or SEQ ID NOs: 453-467;
(xxviii) SEQ ID NO: 28 or SEQ ID NOs: 468-482;
(xxix) SEQ ID NO: 29 or SEQ ID NOs: 483-497;
(xxx) SEQ ID NO: 30 or SEQ ID NOs: 498-512;
(xxxi) SEQ ID NO: 31 or SEQ ID NOs: 513-527;
(xxxii) SEQ ID NO: 32 or SEQ ID NOs: 528-542;
(xxxiii) SEQ ID NO: 33 or SEQ ID NOs: 543-557;
(xxxiv) SEQ ID NO: 34 or SEQ ID NOs: 558-572;
(xxxv) SEQ ID NO: 35 or SEQ ID NOs: 573-587;
(xxxvi) SEQ ID NO: 36 or SEQ ID NOs: 588-602;
(xxxvii) SEQ ID NO: 37 or SEQ ID NOs: 603-617;
(xxxviii) SEQ ID NO: 38 or SEQ ID NOs: 618-632;
(xxxix) SEQ ID NO: 39 or SEQ ID NOs: 633-647;
(xl) SEQ ID NO: 40 or SEQ ID NOs: 648-662;
(xli) SEQ ID NO: 41 or SEQ ID NOs: 667-681;
(xlii) SEQ ID NO: 42 or SEQ ID NOs: 682-696;
(xliii) SEQ ID NO: 43 or SEQ ID NOs: 697-711;
(xliv) SEQ ID NO: 44 or SEQ ID NOs: 712-717;
(xlv) SEQ ID NO: 45 or SEQ ID NOs: 718-723;
(xlvi) SEQ ID NO: 46 or SEQ ID NOs: 724-738;
(xlvii) SEQ ID NO: 47 or SEQ ID NOs: 739-753;
(xlviii) SEQ ID NO: 48 or SEQ ID NOs: 754-768;
(xlix) SEQ ID NO: 49 or SEQ ID NOs: 769-783;
(l) SEQ ID NO: 50 or SEQ ID NOs: 784-798;
(li) SEQ ID NO: 51 or SEQ ID NOs: 799-813;
(lii) SEQ ID NO: 52 or SEQ ID NOs: 814-828;
(liii) SEQ ID NO: 53 or SEQ ID NOs: 829-843;
(liv) SEQ ID NO: 54 or SEQ ID NOs: 844-862;
(lv) SEQ ID NO: 55 or SEQ ID NOs: 863-877;
(lvi) SEQ ID NO: 56 or SEQ ID NOs: 878-892;
(lvii) SEQ ID NO: 57 or SEQ ID NOs: 893-898; and
(lviii) SEQ ID NO: 58 or SEQ ID NOs: 899-913.

10. A pharmaceutical composition comprising the peptide according to claim 1 and a pharmaceutically acceptable carrier, adjuvant or diluent.

11. (canceled)

12. The pharmaceutical composition of claim 10 which is capable of stimulating an immune response.

13.-16. (canceled)

17. A method of treating or reducing symptoms of a disease in a patient in need of treatment thereof, the method comprising administering to the patient a therapeutically effective amount of the peptide according to claim 1.

18. A method for producing a pharmaceutical composition, comprising mixing the peptide of claim 1 with a pharmaceutically acceptable carrier, adjuvant or diluent.

19. A nucleic acid encoding the peptide of claim 1.

20. A cell having integrated in its genome a nucleic acid encoding the peptide of claim 1 operably linked to a transcription control nucleic acid sequence.

21. A nucleic acid expression vector comprising a nucleic acid encoding the peptide of claim 1 operably linked to a transcription control nucleic acid sequence.

22. A cell transfected with the nucleic acid expression vector of claim 21.

23. A method of identifying a peptide that is capable of stimulating an immune response, the method comprising:
testing a candidate peptide for an ability to induce an immune response, said candidate peptide comprising a contiguous amino acid sequence having at least 70% sequence identity to the amino acid sequence set forth in one of SEQ ID NOs:10, 196-210, or one of SEQ ID NOs:11, 211-233, or one of SEQ ID NOs:1-9, 12-195, 234-913, wherein the candidate peptide has an amino acid length of from 8 to 50 amino acids.

24. The method of claim 23 which comprises, prior to the step of testing, chemically modifying the candidate peptide to provide a structurally modified candidate peptide.

25. The method of claim 23 wherein testing comprises contacting the candidate peptide with a population of T cells in vitro and assaying T cell proliferation.

26. The method of claim 23 wherein testing comprises monitoring a population of T cells that has been contacted with the candidate peptide for production of IL-4 and/or IFNγ.

27. The method of claim 17, wherein the disease is an allergic disease.

28. The method of claim 17, wherein the disease is selected from fungal allergy, fungal asthma, fungal infection, SAFS, ABPA, and Aspergillosis

29. The method of claim 17, wherein the disease is an allergic disease caused by an Alternaria alternata protein allergen or by infection of tissue by Alternaria alternata.


SEQUENCE LISTING (1163 sequences)

SEQ ID NO: 1 - protein, 9 aa, Alternaria alternata
FTTIASLFA

SEQ ID NO: 2 - protein, 9 aa, Alternaria alternata
MQFTTIASL

SEQ ID NO: 3 - protein, 9 aa, Alternaria alternata
IASLFAAAG

SEQ ID NO: 4 - protein, 9 aa, Alternaria alternata
LAAAAPLES

SEQ ID NO: 5 - protein, 9 aa, Alternaria alternata
WKISEFYGR

SEQ ID NO: 6 - protein, 9 aa, Alternaria alternata
YYNSLGFNI

SEQ ID NO: 7 - protein, 9 aa, Alternaria alternata
LGFNIKATN

SEQ ID NO: 8 - protein, 9 aa, Alternaria alternata
FNIKATNGG

SEQ ID NO: 9 - protein, 9 aa, Alternaria alternata
IKATNGGTL

SEQ ID NO: 10 - protein, 9 aa, Alternaria alternata
ITYVATATL

SEQ ID NO: 11 - protein, 9 aa, Alternaria alternata
YVATATLPN

SEQ ID NO: 12 - protein, 9 aa, Alternaria alternata
LPNYCRAGG

SEQ ID NO: 13 - protein, 9 aa, Artificial sequence
  Synthetic sequence: Peptide derivative
LPNYVRAGG

SEQ ID NO: 14 - protein, 9 aa, Alternaria alternata
FVCQGVADA

SEQ ID NO: 15 - protein, 9 aa, Artificial sequence
  Synthetic sequence: Peptide derivative
FVVQGVADA

SEQ ID NO: 16 - protein, 9 aa, Alternaria alternata
YITLVTLPK

SEQ ID NO: 17 - protein, 9 aa, Alternaria alternata
ITLVTLPKS

SEQ ID NO: 18 - protein, 9 aa, Alternaria alternata
LFAAAGLAA

SEQ ID NO: 19 - protein, 9 aa, Alternaria alternata
FAAAGLAAA

SEQ ID NO: 20 - protein, 9 aa, Alternaria alternata
YNSLGFNIK

SEQ ID NO: 21 - protein, 9 aa, Alternaria alternata
YCRAGGNGP

SEQ ID NO: 22 - protein, 9 aa, Artificial sequence
  Synthetic sequence: Peptide derivative
YVRAGGNGP

SEQ ID NO: 23 - protein, 9 aa, Alternaria alternata
LDFTCSAQA

SEQ ID NO: 24 - protein, 9 aa, Artificial sequence
  Synthetic sequence: Peptide derivative
LDFTVSAQA

SEQ ID NO: 25 - protein, 9 aa, Alternaria alternata
WYSCGENSF

SEQ ID NO: 26 - protein, 9 aa, Artificial sequence
  Synthetic sequence: Peptide derivative
WYSVGENSF

SEQ ID NO: 27 - protein, 9 aa, Alternaria alternata
VSDDITYVA

SEQ ID NO: 28 - protein, 9 aa, Alternaria alternata
LVNHFTNEF

SEQ ID NO: 29 - protein, 9 aa, Alternaria alternata
ILLLDVAPL

SEQ ID NO: 30 - protein, 9 aa, Alternaria alternata
LRIINEPTA

SEQ ID NO: 31 - protein, 9 aa, Alternaria alternata
LRRLRTACE

SEQ ID NO: 32 - protein, 9 aa, Alternaria alternata
IQGFPTIKL

SEQ ID NO: 33 - protein, 9 aa, Alternaria alternata
VVGLRSGQI

SEQ ID NO: 34 - protein, 9 aa, Alternaria alternata
FVSTGTLGG

SEQ ID NO: 35 - protein, 9 aa, Alternaria alternata
VVIVTGASG

SEQ ID NO: 36 - protein, 9 aa, Alternaria alternata
LVITSSMSG

SEQ ID NO: 37 - protein, 9 aa, Artificial sequence
  Synthetic sequence: Peptide derivative
LVITSSLSG

SEQ ID NO: 38 - protein, 9 aa, Alternaria alternata
IVLLSLCQL

SEQ ID NO: 39 - protein, 9 aa, Artificial sequence
  Synthetic sequence: Peptide derivative
IVLLSLVQL

SEQ ID NO: 40 - protein, 9 aa, Alternaria alternata
FRDDRIEII

SEQ ID NO: 41 - protein, 9 aa, Alternaria alternata
VAMNPVNTV

SEQ ID NO: 42 - protein, 9 aa, Artificial sequence
  Synthetic sequence: Peptide derivative
VALNPVNTV

SEQ ID NO: 43 - protein, 9 aa, Alternaria alternata
LVTIAKVDA

SEQ ID NO: 44 - protein, 9 aa, Alternaria alternata
MKHLAAYLL

SEQ ID NO: 45 - protein, 9 aa, Artificial sequence
  Synthetic sequence: Peptide derivative
LKHLAAYLL

SEQ ID NO: 46 - protein, 9 aa, Alternaria alternata
YQKLKALAK

SEQ ID NO: 47 - protein, 9 aa, Alternaria alternata
LKSCNALLL

SEQ ID NO: 48 - protein, 9 aa, Artificial sequence
  Synthetic sequence: Peptide derivative
LKSVNALLL

SEQ ID NO: 49 - protein, 9 aa, Alternaria alternata
WGVMVSHRS

SEQ ID NO: 50 - protein, 9 aa, Artificial sequence
  Synthetic sequence: Peptide derivative
WGVLVSHRS

SEQ ID NO: 51 - protein, 9 aa, Alternaria alternata
GYKTAFSML

SEQ ID NO: 52 - protein, 9 aa, Artificial sequence
  Synthetic sequence: Peptide derivative
GYKTAFSLL

SEQ ID NO: 53 - protein, 9 aa, Alternaria alternata
FVPQDIQKL

SEQ ID NO: 54 - protein, 9 aa, Alternaria alternata
YVYFASDAS

SEQ ID NO: 55 - protein, 9 aa, Alternaria alternata
YFIEPTIFS

SEQ ID NO: 56 - protein, 9 aa, Alternaria alternata
YIQTKTVSI

SEQ ID NO: 57 - protein, 9 aa, Alternaria alternata
IRLGDVLFG

SEQ ID NO: 58 - protein, 9 aa, Alternaria alternata
WVNSYNTLH

SEQ ID NO: 59 - protein, 11 aa, Artificial sequence
  Synthetic sequence: Peptide derivative
LQFTTIASLFA

SEQ ID NO: 60 - protein, 10 aa, Alternaria alternata
QFTTIASLFA

SEQ ID NO: 61 - protein, 13 aa, Alternaria alternata
FTTIASLFAAAGL

SEQ ID NO: 62 - protein, 12 aa, Alternaria alternata
FTTIASLFAAAG

SEQ ID NO: 63 - protein, 11 aa, Alternaria alternata
FTTIASLFAAA

SEQ ID NO: 64 - protein, 10 aa, Alternaria alternata
FTTIASLFAA

SEQ ID NO: 65 - protein, 15 aa, Artificial sequence
  Synthetic sequence: Peptide derivative
LQFTTIASLFAAAGL

SEQ ID NO: 66 - protein, 14 aa, Alternaria alternata
QFTTIASLFAAAGL

SEQ ID NO: 67 - protein, 13 aa, Alternaria alternata
QFTTIASLFAAAG

SEQ ID NO: 68 - protein, 12 aa, Alternaria alternata
QFTTIASLFAAA

SEQ ID NO: 69 - protein, 11 aa, Alternaria alternata
QFTTIASLFAA

SEQ ID NO: 70 - protein, 14 aa, Artificial sequence
  Synthetic sequence: Peptide derivative
LQFTTIASLFAAAG

SEQ ID NO: 71 - protein, 13 aa, Alternaria alternata
QFTTIASLFAAAG

SEQ ID NO: 72 - protein, 13 aa, Artificial sequence
  Synthetic sequence: Peptide derivative
LQFTTIASLFAAA

SEQ ID NO: 73 - protein, 12 aa, Alternaria alternata
QFTTIASLFAAA

SEQ ID NO: 74 - protein, 12 aa, Artificial sequence
  Synthetic sequence: Peptide derivative
LQFTTIASLFAA

SEQ ID NO: 75 - protein, 11 aa, Alternaria alternata
QFTTIASLFAA

SEQ ID NO: 76 - protein, 11 aa, Alternaria alternata
MQFTTIASLFA

SEQ ID NO: 77 - protein, 15 aa, Alternaria alternata
MQFTTIASLFAAAGL

SEQ ID NO: 78 - protein, 14 aa, Alternaria alternata
MQFTTIASLFAAAG

SEQ ID NO: 79 - protein, 13 aa, Alternaria alternata
MQFTTIASLFAAA

SEQ ID NO: 80 - protein, 12 aa, Alternaria alternata
MQFTTIASLFAA

SEQ ID NO: 81 - protein, 15 aa, Alternaria alternata
MQFTTIASLFAAAGL

SEQ ID NO: 82 - protein, 14 aa, Alternaria alternata
MQFTTIASLFAAAG

SEQ ID NO: 83 - protein, 13 aa, Alternaria alternata
MQFTTIASLFAAA

SEQ ID NO: 84 - protein, 12 aa, Alternaria alternata
MQFTTIASLFAA

SEQ ID NO: 85 - protein, 11 aa, Alternaria alternata
MQFTTIASLFA

SEQ ID NO: 86 - protein, 10 aa, Alternaria alternata
MQFTTIASLF

SEQ ID NO: 87 - protein, 15 aa, Artificial sequence
  Synthetic sequence: Peptide derivative
LQFTTIASLFAAAGL

SEQ ID NO: 88 - protein, 14 aa, Artificial sequence
  Synthetic sequence: Peptide derivative
LQFTTIASLFAAAG

SEQ ID NO: 89 - protein, 13 aa, Artificial sequence
  Synthetic sequence: Peptide derivative
LQFTTIASLFAAA

SEQ ID NO: 90 - protein, 12 aa, Artificial sequence
  Synthetic sequence: Peptide derivative
LQFTTIASLFAA

SEQ ID NO: 91 - protein, 11 aa, Artificial sequence
  Synthetic sequence: Peptide derivative
LQFTTIASLFA

SEQ ID NO: 92 - protein, 10 aa, Artificial sequence
  Synthetic sequence: Peptide derivative
LQFTTIASLF

SEQ ID NO: 93 - protein, 15 aa, Artificial sequence
  Synthetic sequence: Peptide derivative
LQFTTIASLFAAAGL

SEQ ID NO: 94 - protein, 14 aa, Artificial sequence
  Synthetic sequence: Peptide derivative
LQFTTIASLFAAAG

SEQ ID NO: 95 - protein, 13 aa, Alternaria alternata
QFTTIASLFAAAG

SEQ ID NO: 96 - protein, 12 aa, Alternaria alternata
FTTIASLFAAAG

SEQ ID NO: 97 - protein, 11 aa, Alternaria alternata
TTIASLFAAAG

SEQ ID NO: 98 - protein, 10 aa, Alternaria alternata
TIASLFAAAG

SEQ ID NO: 99 - protein, 14 aa, Alternaria alternata
QFTTIASLFAAAGL

SEQ ID NO: 100 - protein, 13 aa, Alternaria alternata
FTTIASLFAAAGL

SEQ ID NO: 101 - protein, 12 aa, Alternaria alternata
TTIASLFAAAGL

SEQ ID NO: 102 - protein, 11 aa, Alternaria alternata
TIASLFAAAGL

SEQ ID NO: 103 - protein, 10 aa, Alternaria alternata
IASLFAAAGL

SEQ ID NO: 104 - protein, 15 aa, Alternaria alternata
MQFTTIASLFAAAGL

SEQ ID NO: 105 - protein, 14 aa, Alternaria alternata
MQFTTIASLFAAAG

SEQ ID NO: 106 - protein, 12 aa, Alternaria alternata
AAGLAAAAPLES

SEQ ID NO: 107 - protein, 11 aa, Alternaria alternata
AGLAAAAPLES

SEQ ID NO: 108 - protein, 10 aa, Alternaria alternata
GLAAAAPLES

SEQ ID NO: 109 - protein, 12 aa, Alternaria alternata
LAAAAPLESRQD

SEQ ID NO: 110 - protein, 11 aa, Alternaria alternata
LAAAAPLESRQ

SEQ ID NO: 111 - protein, 10 aa, Alternaria alternata
LAAAAPLESR

SEQ ID NO: 112 - protein, 15 aa, Alternaria alternata
AAGLAAAAPLESRQD

SEQ ID NO: 113 - protein, 14 aa, Alternaria alternata
AGLAAAAPLESRQD

SEQ ID NO: 114 - protein, 13 aa, Alternaria alternata
GLAAAAPLESRQD

SEQ ID NO: 115 - protein, 14 aa, Alternaria alternata
AAGLAAAAPLESRQ

SEQ ID NO: 116 - protein, 13 aa, Alternaria alternata
AGLAAAAPLESRQ

SEQ ID NO: 117 - protein, 12 aa, Alternaria alternata
GLAAAAPLESRQ

SEQ ID NO: 118 - protein, 13 aa, Alternaria alternata
AAGLAAAAPLESR

SEQ ID NO: 119 - protein, 12 aa, Alternaria alternata
AGLAAAAPLESR

SEQ ID NO: 120 - protein, 11 aa, Alternaria alternata
GLAAAAPLESR

SEQ ID NO: 121 - protein, 12 aa, Alternaria alternata
DYVWKISEFYGR

SEQ ID NO: 122 - protein, 11 aa, Alternaria alternata
YVWKISEFYGR

SEQ ID NO: 123 - protein, 10 aa, Alternaria alternata
VWKISEFYGR

SEQ ID NO: 124 - protein, 12 aa, Alternaria alternata
WKISEFYGRKPE

SEQ ID NO: 125 - protein, 11 aa, Alternaria alternata
WKISEFYGRKP

SEQ ID NO: 126 - protein, 10 aa, Alternaria alternata
WKISEFYGRK

SEQ ID NO: 127 - protein, 15 aa, Alternaria alternata
DYVWKISEFYGRKPE

SEQ ID NO: 128 - protein, 14 aa, Alternaria alternata
DYVWKISEFYGRKP

SEQ ID NO: 129 - protein, 13 aa, Alternaria alternata
DYVWKISEFYGRK

SEQ ID NO: 130 - protein, 14 aa, Alternaria alternata
YVWKISEFYGRKPE

SEQ ID NO: 131 - protein, 13 aa, Alternaria alternata
YVWKISEFYGRKP

SEQ ID NO: 132 - protein, 12 aa, Alternaria alternata
YVWKISEFYGRK

SEQ ID NO: 133 - protein, 13 aa, Alternaria alternata
VWKISEFYGRKPE

SEQ ID NO: 134 - protein, 12 aa, Alternaria alternata
VWKISEFYGRKP

SEQ ID NO: 135 - protein, 11 aa, Alternaria alternata
VWKISEFYGRK

SEQ ID NO: 136 - protein, 12 aa, Alternaria alternata
EGTYYNSLGFNI

SEQ ID NO: 137 - protein, 11 aa, Alternaria alternata
GTYYNSLGFNI

SEQ ID NO: 138 - protein, 10 aa, Alternaria alternata
TYYNSLGFNI

SEQ ID NO: 139 - protein, 12 aa, Alternaria alternata
YYNSLGFNIKAT

SEQ ID NO: 140 - protein, 11 aa, Alternaria alternata
YYNSLGFNIKA

SEQ ID NO: 141 - protein, 10 aa, Alternaria alternata
YYNSLGFNIK

SEQ ID NO: 142 - protein, 15 aa, Alternaria alternata
EGTYYNSLGFNIKAT

SEQ ID NO: 143 - protein, 14 aa, Alternaria alternata
EGTYYNSLGFNIKA

SEQ ID NO: 144 - protein, 13 aa, Alternaria alternata
EGTYYNSLGFNIK

SEQ ID NO: 145 - protein, 14 aa, Alternaria alternata
GTYYNSLGFNIKAT

SEQ ID NO: 146 - protein, 13 aa, Alternaria alternata
GTYYNSLGFNIKA

SEQ ID NO: 147 - protein, 12 aa, Alternaria alternata
GTYYNSLGFNIK

SEQ ID NO: 148 - protein, 13 aa, Alternaria alternata
TYYNSLGFNIKAT

SEQ ID NO: 149 - protein, 12 aa, Alternaria alternata
TYYNSLGFNIKA

SEQ ID NO: 150 - protein, 11 aa, Alternaria alternata
TYYNSLGFNIK

SEQ ID NO: 151 - protein, 12 aa, Alternaria alternata
YNSLGFNIKATN

SEQ ID NO: 152 - protein, 11 aa, Alternaria alternata
NSLGFNIKATN

SEQ ID NO: 153 - protein, 10 aa, Alternaria alternata
SLGFNIKATN

SEQ ID NO: 154 - protein, 12 aa, Alternaria alternata
LGFNIKATNGGT

SEQ ID NO: 155 - protein, 11 aa, Alternaria alternata
LGFNIKATNGG

SEQ ID NO: 156 - protein, 10 aa, Alternaria alternata
LGFNIKATNG

SEQ ID NO: 157 - protein, 15 aa, Alternaria alternata
YNSLGFNIKATNGGT

SEQ ID NO: 158 - protein, 14 aa, Alternaria alternata
YNSLGFNIKATNGG

SEQ ID NO: 159 - protein, 13 aa, Alternaria alternata
YNSLGFNIKATNG

SEQ ID NO: 160 - protein, 14 aa, Alternaria alternata
NSLGFNIKATNGGT

SEQ ID NO: 161 - protein, 13 aa, Alternaria alternata
NSLGFNIKATNGG

SEQ ID NO: 162 - protein, 12 aa, Alternaria alternata
NSLGFNIKATNG

SEQ ID NO: 163 - protein, 13 aa, Alternaria alternata
SLGFNIKATNGGT

SEQ ID NO: 164 - protein, 12 aa, Alternaria alternata
SLGFNIKATNGG

SEQ ID NO: 165 - protein, 11 aa, Alternaria alternata
SLGFNIKATNG

SEQ ID NO: 166 - protein, 12 aa, Alternaria alternata
SLGFNIKATNGG

SEQ ID NO: 167 - protein, 11 aa, Alternaria alternata
LGFNIKATNGG

SEQ ID NO: 168 - protein, 10 aa, Alternaria alternata
GFNIKATNGG

SEQ ID NO: 169 - protein, 12 aa, Alternaria alternata
FNIKATNGGTLD

SEQ ID NO: 170 - protein, 11 aa, Alternaria alternata
FNIKATNGGTL

SEQ ID NO: 171 - protein, 10 aa, Alternaria alternata
FNIKATNGGT

SEQ ID NO: 172 - protein, 15 aa, Alternaria alternata
SLGFNIKATNGGTLD

SEQ ID NO: 173 - protein, 14 aa, Alternaria alternata
SLGFNIKATNGGTL

SEQ ID NO: 174 - protein, 13 aa, Alternaria alternata
SLGFNIKATNGGT

SEQ ID NO: 175 - protein, 14 aa, Alternaria alternata
LGFNIKATNGGTLD

SEQ ID NO: 176 - protein, 13 aa, Alternaria alternata
LGFNIKATNGGTL

SEQ ID NO: 177 - protein, 12 aa, Alternaria alternata
LGFNIKATNGGT

SEQ ID NO: 178 - protein, 13 aa, Alternaria alternata
GFNIKATNGGTLD

SEQ ID NO: 179 - protein, 12 aa, Alternaria alternata
GFNIKATNGGTL

SEQ ID NO: 180 - protein, 11 aa, Alternaria alternata
GFNIKATNGGT

SEQ ID NO: 181 - protein, 12 aa, Alternaria alternata
GFNIKATNGGTL

SEQ ID NO: 182 - protein, 11 aa, Alternaria alternata
FNIKATNGGTL

SEQ ID NO: 183 - protein, 10 aa, Alternaria alternata
NIKATNGGTL

SEQ ID NO: 184 - protein, 12 aa, Alternaria alternata
IKATNGGTLDFT

SEQ ID NO: 185 - protein, 11 aa, Alternaria alternata
IKATNGGTLDF

SEQ ID NO: 186 - protein, 10 aa, Alternaria alternata
IKATNGGTLD

SEQ ID NO: 187 - protein, 15 aa, Alternaria alternata
GFNIKATNGGTLDFT

SEQ ID NO: 188 - protein, 14 aa, Alternaria alternata
GFNIKATNGGTLDF

SEQ ID NO: 189 - protein, 13 aa, Alternaria alternata
GFNIKATNGGTLD

SEQ ID NO: 190 - protein, 14 aa, Alternaria alternata
FNIKATNGGTLDFT

SEQ ID NO: 191 - protein, 13 aa, Alternaria alternata
FNIKATNGGTLDF

SEQ ID NO: 192 - protein, 12 aa, Alternaria alternata
FNIKATNGGTLD

SEQ ID NO: 193 - protein, 13 aa, Alternaria alternata
NIKATNGGTLDFT

SEQ ID NO: 194 - protein, 12 aa, Alternaria alternata
NIKATNGGTLDF

SEQ ID NO: 195 - protein, 11 aa, Alternaria alternata
NIKATNGGTLD

SEQ ID NO: 196 - protein, 12 aa, Alternaria alternata
SDDITYVATATL

SEQ ID NO: 197 - protein, 11 aa, Alternaria alternata
DDITYVATATL

SEQ ID NO: 198 - protein, 10 aa, Alternaria alternata
DITYVATATL

SEQ ID NO: 199 - protein, 12 aa, Alternaria alternata
ITYVATATLPNY

SEQ ID NO: 200 - protein, 11 aa, Alternaria alternata
ITYVATATLPN

SEQ ID NO: 201 - protein, 10 aa, Alternaria alternata
ITYVATATLP

SEQ ID NO: 202 - protein, 15 aa, Alternaria alternata
SDDITYVATATLPNY

SEQ ID NO: 203 - protein, 14 aa, Alternaria alternata
SDDITYVATATLPN

SEQ ID NO: 204 - protein, 13 aa, Alternaria alternata
SDDITYVATATLP

SEQ ID NO: 205 - protein, 14 aa, Alternaria alternata
DDITYVATATLPNY

SEQ ID NO: 206 - protein, 13 aa, Alternaria alternata
DDITYVATATLPN

SEQ ID NO: 207 - protein, 12 aa, Alternaria alternata
DDITYVATATLP

SEQ ID NO: 208 - protein, 13 aa, Alternaria alternata
DITYVATATLPNY

SEQ ID NO: 209 - protein, 12 aa, Alternaria alternata
DITYVATATLPN

SEQ ID NO: 210 - protein, 11 aa, Alternaria alternata
DITYVATATLP

SEQ ID NO: 211 - protein, 12 aa, Alternaria alternata
DITYVATATLPN

SEQ ID NO: 212 - protein, 11 aa, Alternaria alternata
ITYVATATLPN

SEQ ID NO: 213 - protein, 10 aa, Alternaria alternata
TYVATATLPN

SEQ ID NO: 214 - protein, 12 aa, Artificial sequence
  Synthetic sequence: Peptide derivative
YVATATLPNYVR

SEQ ID NO: 215 - protein, 11 aa, Artificial sequence
  Synthetic sequence: Peptide derivative
YVATATLPNYV

SEQ ID NO: 216 - protein, 10 aa, Alternaria alternata
YVATATLPNY

SEQ ID NO: 217 - protein, 15 aa, Artificial sequence
  Synthetic sequence: Peptide derivative
DITYVATATLPNYVR

SEQ ID NO: 218 - protein, 14 aa, Artificial sequence
  Synthetic sequence: Peptide derivative
DITYVATATLPNYV

SEQ ID NO: 219 - protein, 13 aa, Alternaria alternata
DITYVATATLPNY

SEQ ID NO: 220 - protein, 14 aa, Artificial sequence
  Synthetic sequence: Peptide derivative
ITYVATATLPNYVR

SEQ ID NO: 221 - protein, 13 aa, Artificial sequence
  Synthetic sequence: Peptide derivative
ITYVATATLPNYV

SEQ ID NO: 222 - protein, 12 aa, Alternaria alternata
ITYVATATLPNY

SEQ ID NO: 223 - protein, 13 aa, Artificial sequence
  Synthetic sequence: Peptide derivative
TYVATATLPNYVR

SEQ ID NO: 224 - protein, 12 aa, Artificial sequence
  Synthetic sequence: Peptide derivative
TYVATATLPNYV

SEQ ID NO: 225 - protein, 11 aa, Alternaria alternata
TYVATATLPNY

SEQ ID NO: 226 - protein, 12 aa, Alternaria alternata
YVATATLPNYCR

SEQ ID NO: 227 - protein, 11 aa, Alternaria alternata
YVATATLPNYC

SEQ ID NO: 228 - protein, 15 aa, Alternaria alternata
DITYVATATLPNYCR

SEQ ID NO: 229 - protein, 14 aa, Alternaria alternata
DITYVATATLPNYC

SEQ ID NO: 230 - protein, 14 aa, Alternaria alternata
ITYVATATLPNYCR

SEQ ID NO: 231 - protein, 13 aa, Alternaria alternata
ITYVATATLPNYC

SEQ ID NO: 232 - protein, 13 aa, Alternaria alternata
TYVATATLPNYCR

SEQ ID NO: 233 - protein, 12 aa, Alternaria alternata
TYVATATLPNYC

SEQ ID NO: 234 - protein, 12 aa, Alternaria alternata
TATLPNYCRAGG

SEQ ID NO: 235 - protein, 11 aa, Alternaria alternata
ATLPNYCRAGG

SEQ ID NO: 236 - protein, 10 aa, Alternaria alternata
TLPNYCRAGG

SEQ ID NO: 237 - protein, 12 aa, Alternaria alternata
LPNYCRAGGNGP

SEQ ID NO: 238 - protein, 11 aa, Alternaria alternata
LPNYCRAGGNG

SEQ ID NO: 239 - protein, 10 aa, Alternaria alternata
LPNYCRAGGN

SEQ ID NO: 240 - protein, 15 aa, Alternaria alternata
TATLPNYCRAGGNGP

SEQ ID NO: 241 - protein, 14 aa, Alternaria alternata
TATLPNYCRAGGNG

SEQ ID NO: 242 - protein, 13 aa, Alternaria alternata
TATLPNYCRAGGN

SEQ ID NO: 243 - protein, 14 aa, Alternaria alternata
ATLPNYCRAGGNGP

SEQ ID NO: 244 - protein, 13 aa, Alternaria alternata
ATLPNYCRAGGNG

SEQ ID NO: 245 - protein, 12 aa, Alternaria alternata
ATLPNYCRAGGN

SEQ ID NO: 246 - protein, 13 aa, Alternaria alternata
TLPNYCRAGGNGP

SEQ ID NO: 247 - protein, 12 aa, Alternaria alternata
TLPNYCRAGGNG

SEQ ID NO: 248 - protein, 11 aa, Alternaria alternata
TLPNYCRAGGN

SEQ ID NO: 249 - protein, 12 aa, Artificial sequence
  Synthetic sequence: Peptide derivative
TATLPNYVRAGG

SEQ ID NO: 250 - protein, 11 aa, Artificial sequence
  Synthetic sequence: Peptide derivative
ATLPNYVRAGG

SEQ ID NO: 251 - protein, 10 aa, Artificial sequence
  Synthetic sequence: Peptide derivative
TLPNYVRAGG

SEQ ID NO: 252 - protein, 12 aa, Artificial sequence
  Synthetic sequence: Peptide derivative
LPNYVRAGGNGP

SEQ ID NO: 253 - protein, 11 aa, Artificial sequence
  Synthetic sequence: Peptide derivative
LPNYVRAGGNG

SEQ ID NO: 254 - protein, 10 aa, Artificial sequence
  Synthetic sequence: Peptide derivative
LPNYVRAGGN

SEQ ID NO: 255 - protein, 15 aa, Artificial sequence
  Synthetic sequence: Peptide derivative
TATLPNYVRAGGNGP

SEQ ID NO: 256 - protein, 14 aa, Artificial sequence
  Synthetic sequence: Peptide derivative
TATLPNYVRAGGNG

SEQ ID NO: 257 - protein, 13 aa, Artificial sequence
  Synthetic sequence: Peptide derivative
TATLPNYVRAGGN

SEQ ID NO: 258 - protein, 14 aa, Artificial sequence
  Synthetic sequence: Peptide derivative
ATLPNYVRAGGNGP

SEQ ID NO: 259 - protein, 13 aa, Artificial sequence
  Synthetic sequence: Peptide derivative
ATLPNYVRAGGNG

SEQ ID NO: 260 - protein, 12 aa, Artificial sequence
  Synthetic sequence: Peptide derivative
ATLPNYVRAGGN

SEQ ID NO: 261 - protein, 13 aa, Artificial sequence
  Synthetic sequence: Peptide derivative
TLPNYVRAGGNGP

SEQ ID NO: 262 - protein, 12 aa, Artificial sequence
  Synthetic sequence: Peptide derivative
TLPNYVRAGGNG

SEQ ID NO: 263 - protein, 11 aa, Artificial sequence
  Synthetic sequence: Peptide derivative
TLPNYVRAGGN

SEQ ID NO: 264 - protein, 12 aa, Alternaria alternata
PKDFVCQGVADA

SEQ ID NO: 265 - protein, 11 aa, Alternaria alternata
KDFVCQGVADA

SEQ ID NO: 266 - protein, 10 aa, Alternaria alternata
DFVCQGVADA

SEQ ID NO: 267 - protein, 12 aa, Alternaria alternata
FVCQGVADAYIT

SEQ ID NO: 268 - protein, 11 aa, Alternaria alternata
FVCQGVADAYI

SEQ ID NO: 269 - protein, 10 aa, Alternaria alternata
FVCQGVADAY

SEQ ID NO: 270 - protein, 15 aa, Alternaria alternata
PKDFVCQGVADAYIT

SEQ ID NO: 271 - protein, 14 aa, Alternaria alternata
PKDFVCQGVADAYI

SEQ ID NO: 272 - protein, 13 aa, Alternaria alternata
PKDFVCQGVADAY

SEQ ID NO: 273 - protein, 14 aa, Alternaria alternata
KDFVCQGVADAYIT

SEQ ID NO: 274 - protein, 13 aa, Alternaria alternata
KDFVCQGVADAYI

SEQ ID NO: 275 - protein, 12 aa, Alternaria alternata
KDFVCQGVADAY

SEQ ID NO: 276 - protein, 13 aa, Alternaria alternata
DFVCQGVADAYIT

SEQ ID NO: 277 - protein, 12 aa, Alternaria alternata
DFVCQGVADAYI

SEQ ID NO: 278 - protein, 11 aa, Alternaria alternata
DFVCQGVADAY

SEQ ID NO: 279 - protein, 12 aa, Artificial sequence
  Synthetic sequence: Peptide derivative
PKDFVVQGVADA

SEQ ID NO: 280 - protein, 11 aa, Artificial sequence
  Synthetic sequence: Peptide derivative
KDFVVQGVADA

SEQ ID NO: 281 - protein, 10 aa, Artificial sequence
  Synthetic sequence: Peptide derivative
DFVVQGVADA

SEQ ID NO: 282 - protein, 12 aa, Artificial sequence
  Synthetic sequence: Peptide derivative
FVVQGVADAYIT

SEQ ID NO: 283 - protein, 11 aa, Artificial sequence
  Synthetic sequence: Peptide derivative
FVVQGVADAYI

SEQ ID NO: 284 - protein, 10 aa, Artificial sequence
  Synthetic sequence: Peptide derivative
FVVQGVADAY

SEQ ID NO: 285 - protein, 15 aa, Artificial sequence
  Synthetic sequence: Peptide derivative
PKDFVVQGVADAYIT

SEQ ID NO: 286 - protein, 14 aa, Artificial sequence
  Synthetic sequence: Peptide derivative
PKDFVVQGVADAYI

SEQ ID NO: 287 - protein, 13 aa, Artificial sequence
  Synthetic sequence: Peptide derivative
PKDFVVQGVADAY

SEQ ID NO: 288 - protein, 14 aa, Artificial sequence
  Synthetic sequence: Peptide derivative
KDFVVQGVADAYIT

SEQ ID NO: 289 - protein, 13 aa, Artificial sequence
  Synthetic sequence: Peptide derivative
KDFVVQGVADAYI

SEQ ID NO: 290 - protein, 12 aa, Artificial sequence
  Synthetic sequence: Peptide derivative
KDFVVQGVADAY

SEQ ID NO: 291 - protein, 13 aa, Artificial sequence
  Synthetic sequence: Peptide derivative
DFVVQGVADAYIT

SEQ ID NO: 292 - protein, 12 aa, Artificial sequence
  Synthetic sequence: Peptide derivative
DFVVQGVADAYI

SEQ ID NO: 293 - protein, 11 aa, Artificial sequence
  Synthetic sequence: Peptide derivative
DFVVQGVADAY

SEQ ID NO: 294 - protein, 13 aa, Alternaria alternata
VADAYITLVTLPK

SEQ ID NO: 295 - protein, 12 aa, Alternaria alternata
ADAYITLVTLPK

SEQ ID NO: 296 - protein, 11 aa, Alternaria alternata
DAYITLVTLPK

SEQ ID NO: 297 - protein, 10 aa, Alternaria alternata
AYITLVTLPK

SEQ ID NO: 298 - protein, 11 aa, Alternaria alternata
YITLVTLPKSS

SEQ ID NO: 299 - protein, 10 aa, Alternaria alternata
YITLVTLPKS

SEQ ID NO: 300 - protein, 15 aa, Alternaria alternata
VADAYITLVTLPKSS

SEQ ID NO: 301 - protein, 14 aa, Alternaria alternata
VADAYITLVTLPKS

SEQ ID NO: 302 - protein, 14 aa, Alternaria alternata
ADAYITLVTLPKSS

SEQ ID NO: 303 - protein, 13 aa, Alternaria alternata
ADAYITLVTLPKS

SEQ ID NO: 304 - protein, 13 aa, Alternaria alternata
DAYITLVTLPKSS

SEQ ID NO: 305 - protein, 12 aa, Alternaria alternata
DAYITLVTLPKS

SEQ ID NO: 306 - protein, 12 aa, Alternaria alternata
AYITLVTLPKSS

SEQ ID NO: 307 - protein, 11 aa, Alternaria alternata
AYITLVTLPKS

SEQ ID NO: 308 - protein, 14 aa, Alternaria alternata
VADAYITLVTLPKS

SEQ ID NO: 309 - protein, 13 aa, Alternaria alternata
ADAYITLVTLPKS

SEQ ID NO: 310 - protein, 12 aa, Alternaria alternata
DAYITLVTLPKS

SEQ ID NO: 311 - protein, 11 aa, Alternaria alternata
AYITLVTLPKS

SEQ ID NO: 312 - protein, 10 aa, Alternaria alternata
YITLVTLPKS

SEQ ID NO: 313 - protein, 15 aa, Alternaria alternata
VADAYITLVTLPKSS

SEQ ID NO: 314 - protein, 14 aa, Alternaria alternata
ADAYITLVTLPKSS

SEQ ID NO: 315 - protein, 13 aa, Alternaria alternata
DAYITLVTLPKSS

SEQ ID NO: 316 - protein, 12 aa, Alternaria alternata
AYITLVTLPKSS

SEQ ID NO: 317 - protein, 11 aa, Alternaria alternata
YITLVTLPKSS

SEQ ID NO: 318 - protein, 12 aa, Alternaria alternata
IASLFAAAGLAA

SEQ ID NO: 319 - protein, 11 aa, Alternaria alternata
ASLFAAAGLAA

SEQ ID NO: 320 - protein, 10 aa, Alternaria alternata
SLFAAAGLAA

SEQ ID NO: 321 - protein, 12 aa, Alternaria alternata
LFAAAGLAAAAP

SEQ ID NO: 322 - protein, 11 aa, Alternaria alternata
LFAAAGLAAAA

SEQ ID NO: 323 - protein, 10 aa, Alternaria alternata
LFAAAGLAAA

SEQ ID NO: 324 - protein, 15 aa, Alternaria alternata
IASLFAAAGLAAAAP

SEQ ID NO: 325 - protein, 14 aa, Alternaria alternata
IASLFAAAGLAAAA

SEQ ID NO: 326 - protein, 13 aa, Alternaria alternata
IASLFAAAGLAAA

SEQ ID NO: 327 - protein, 14 aa, Alternaria alternata
ASLFAAAGLAAAAP

SEQ ID NO: 328 - protein, 13 aa, Alternaria alternata
ASLFAAAGLAAAA

SEQ ID NO: 329 - protein, 12 aa, Alternaria alternata
ASLFAAAGLAAA

SEQ ID NO: 330 - protein, 13 aa, Alternaria alternata
SLFAAAGLAAAAP

SEQ ID NO: 331 - protein, 12 aa, Alternaria alternata
SLFAAAGLAAAA

SEQ ID NO: 332 - protein, 11 aa, Alternaria alternata
SLFAAAGLAAA

SEQ ID NO: 333 - protein, 12 aa, Alternaria alternata
ASLFAAAGLAAA

SEQ ID NO: 334 - protein, 11 aa, Alternaria alternata
SLFAAAGLAAA

SEQ ID NO: 335 - protein, 10 aa, Alternaria alternata
LFAAAGLAAA

SEQ ID NO: 336 - protein, 12 aa, Alternaria alternata
FAAAGLAAAAPL

SEQ ID NO: 337 - protein, 11 aa, Alternaria alternata
FAAAGLAAAAP

SEQ ID NO: 338 - protein, 10 aa, Alternaria alternata
FAAAGLAAAA

SEQ ID NO: 339 - protein, 15 aa, Alternaria alternata
ASLFAAAGLAAAAPL

SEQ ID NO: 340 - protein, 14 aa, Alternaria alternata
ASLFAAAGLAAAAP

SEQ ID NO: 341 - protein, 13 aa, Alternaria alternata
ASLFAAAGLAAAA

SEQ ID NO: 342 - protein, 14 aa, Alternaria alternata
SLFAAAGLAAAAPL

SEQ ID NO: 343 - protein, 13 aa, Alternaria alternata
SLFAAAGLAAAAP

SEQ ID NO: 344 - protein, 12 aa, Alternaria alternata
SLFAAAGLAAAA

SEQ ID NO: 345 - protein, 13 aa, Alternaria alternata
LFAAAGLAAAAPL

SEQ ID NO: 346 - protein, 12 aa, Alternaria alternata
LFAAAGLAAAAP

SEQ ID NO: 347 - protein, 11 aa, Alternaria alternata
LFAAAGLAAAA

SEQ ID NO: 348 - protein, 12 aa, Alternaria alternata
GTYYNSLGFNIK

SEQ ID NO: 349 - protein, 11 aa, Alternaria alternata
TYYNSLGFNIK

SEQ ID NO: 350 - protein, 10 aa, Alternaria alternata
YYNSLGFNIK

SEQ ID NO: 351 - protein, 12 aa, Alternaria alternata
YNSLGFNIKATN

SEQ ID NO: 352 - protein, 11 aa, Alternaria alternata
YNSLGFNIKAT

SEQ ID NO: 353 - protein, 10 aa, Alternaria alternata
YNSLGFNIKA

SEQ ID NO: 354 - protein, 15 aa, Alternaria alternata
GTYYNSLGFNIKATN

SEQ ID NO: 355 - protein, 14 aa, Alternaria alternata
GTYYNSLGFNIKAT

SEQ ID NO: 356 - protein, 13 aa, Alternaria alternata
GTYYNSLGFNIKA

SEQ ID NO: 357 - protein, 14 aa, Alternaria alternata
TYYNSLGFNIKATN

SEQ ID NO: 358 - protein, 13 aa, Alternaria alternata
TYYNSLGFNIKAT

SEQ ID NO: 359 - protein, 12 aa, Alternaria alternata
TYYNSLGFNIKA

SEQ ID NO: 360 - protein, 13 aa, Alternaria alternata
YYNSLGFNIKATN

SEQ ID NO: 361 - protein, 12 aa, Alternaria alternata
YYNSLGFNIKAT

SEQ ID NO: 362 - protein, 11 aa, Alternaria alternata
YYNSLGFNIKA

SEQ ID NO: 363 - protein, 12 aa, Alternaria alternata
LPNYCRAGGNGP

SEQ ID NO: 364 - protein, 11 aa, Alternaria alternata
PNYCRAGGNGP

SEQ ID NO: 365 - protein, 10 aa, Alternaria alternata
NYCRAGGNGP

SEQ ID NO: 366 - protein, 12 aa, Alternaria alternata
YCRAGGNGPKDF

SEQ ID NO: 367 - protein, 11 aa, Alternaria alternata
YCRAGGNGPKD

SEQ ID NO: 368 - protein, 10 aa, Alternaria alternata
YCRAGGNGPK

SEQ ID NO: 369 - protein, 15 aa, Alternaria alternata
LPNYCRAGGNGPKDF

SEQ ID NO: 370 - protein, 14 aa, Alternaria alternata
LPNYCRAGGNGPKD

SEQ ID NO: 371 - protein, 13 aa, Alternaria alternata
LPNYCRAGGNGPK

SEQ ID NO: 372 - protein, 14 aa, Alternaria alternata
PNYCRAGGNGPKDF

SEQ ID NO: 373 - protein, 13 aa, Alternaria alternata
PNYCRAGGNGPKD

SEQ ID NO: 374 - protein, 12 aa, Alternaria alternata
PNYCRAGGNGPK

SEQ ID NO: 375 - protein, 13 aa, Alternaria alternata
NYCRAGGNGPKDF

SEQ ID NO: 376 - protein, 12 aa, Alternaria alternata
NYCRAGGNGPKD

SEQ ID NO: 377 - protein, 11 aa, Alternaria alternata
NYCRAGGNGPK

SEQ ID NO: 378 - protein, 12 aa, Artificial sequence
  Synthetic sequence: Peptide derivative
LPNYVRAGGNGP

SEQ ID NO: 379 - protein, 11 aa, Artificial sequence
  Synthetic sequence: Peptide derivative
PNYVRAGGNGP

SEQ ID NO: 380 - protein, 10 aa, Artificial sequence
  Synthetic sequence: Peptide derivative
NYVRAGGNGP

SEQ ID NO: 381 - protein, 12 aa, Artificial sequence
  Synthetic sequence: Peptide derivative
YVRAGGNGPKDF

SEQ ID NO: 382 - protein, 11 aa, Artificial sequence
  Synthetic sequence: Peptide derivative
YVRAGGNGPKD

SEQ ID NO: 383 - protein, 10 aa, Artificial sequence
  Synthetic sequence: Peptide derivative
YVRAGGNGPK

SEQ ID NO: 384 - protein, 15 aa, Artificial sequence
  Synthetic sequence: Peptide derivative
LPNYVRAGGNGPKDF

SEQ ID NO: 385 - protein, 14 aa, Artificial sequence
  Synthetic sequence: Peptide derivative
LPNYVRAGGNGPKD

SEQ ID NO: 386 - protein, 13 aa, Artificial sequence
  Synthetic sequence: Peptide derivative
LPNYVRAGGNGPK

SEQ ID NO: 387 - protein, 14 aa, Artificial sequence
  Synthetic sequence: Peptide derivative
PNYVRAGGNGPKDF

SEQ ID NO: 388 - protein, 13 aa, Artificial sequence
  Synthetic sequence: Peptide derivative
PNYVRAGGNGPKD

SEQ ID NO: 389 - protein, 12 aa, Artificial sequence
  Synthetic sequence: Peptide derivative
PNYVRAGGNGPK

SEQ ID NO: 390 - protein, 13 aa, Artificial sequence
  Synthetic sequence: Peptide derivative
NYVRAGGNGPKDF

SEQ ID NO: 391 - protein, 12 aa, Artificial sequence
  Synthetic sequence: Peptide derivative
NYVRAGGNGPKD

SEQ ID NO: 392 - protein, 11 aa, Artificial sequence
  Synthetic sequence: Peptide derivative
NYVRAGGNGPK

SEQ ID NO: 393 - protein, 12 aa, Alternaria alternata
GGTLDFTCSAQA

SEQ ID NO: 394 - protein, 12 aa, Alternaria alternata
GTLDFTVCSAQA

SEQ ID NO: 395 - protein, 10 aa, Alternaria alternata
TLDFTCSAQA

SEQ ID NO: 396 - protein, 12 aa, Alternaria alternata
LDFTCSAQADKL

SEQ ID NO: 397 - protein, 11 aa, Alternaria alternata
LDFTCSAQADK

SEQ ID NO: 398 - protein, 10 aa, Alternaria alternata
LDFTCSAQAD

SEQ ID NO: 399 - protein, 15 aa, Alternaria alternata
GGTLDFTCSAQADKL

SEQ ID NO: 400 - protein, 14 aa, Alternaria alternata
GGTLDFTCSAQADK

SEQ ID NO: 401 - protein, 13 aa, Alternaria alternata
GGTLDFTCSAQAD

SEQ ID NO: 402 - protein, 14 aa, Alternaria alternata
GTLDFTCSAQADKL

SEQ ID NO: 403 - protein, 13 aa, Alternaria alternata
GTLDFTCSAQADK

SEQ ID NO: 404 - protein, 12 aa, Alternaria alternata
GTLDFTCSAQAD

SEQ ID NO: 405 - protein, 13 aa, Alternaria alternata
TLDFTCSAQADKL

SEQ ID NO: 406 - protein, 12 aa, Alternaria alternata
TLDFTCSAQADK

SEQ ID NO: 407 - protein, 11 aa, Alternaria alternata
TLDFTCSAQAD

SEQ ID NO: 408 - protein, 12 aa, Artificial sequence
  Synthetic sequence: Peptide derivative
GGTLDFTVSAQA

SEQ ID NO: 409 - protein, 11 aa, Artificial sequence
  Synthetic sequence: Peptide derivative
GTLDFTVSAQA

SEQ ID NO: 410 - protein, 10 aa, Artificial sequence
  Synthetic sequence: Peptide derivative
TLDFTVSAQA

SEQ ID NO: 411 - protein, 12 aa, Artificial sequence
  Synthetic sequence: Peptide derivative
LDFTVSAQADKL

SEQ ID NO: 412 - protein, 11 aa, Artificial sequence
  Synthetic sequence: Peptide derivative
LDFTVSAQADK

SEQ ID NO: 413 - protein, 10 aa, Artificial sequence
  Synthetic sequence: Peptide derivative
LDFTVSAQAD

SEQ ID NO: 414 - protein, 15 aa, Artificial sequence
  Synthetic sequence: Peptide derivative
GGTLDFTVSAQADKL

SEQ ID NO: 415 - protein, 14 aa, Artificial sequence
  Synthetic sequence: Peptide derivative
GGTLDFTVSAQADK

SEQ ID NO: 416 - protein, 13 aa, Artificial sequence
  Synthetic sequence: Peptide derivative
GGTLDFTVSAQAD

SEQ ID NO: 417 - protein, 14 aa, Artificial sequence
  Synthetic sequence: Peptide derivative
GTLDFTVSAQADKL

SEQ ID NO: 418 - protein, 13 aa, Artificial sequence
  Synthetic sequence: Peptide derivative
GTLDFTVSAQADK

SEQ ID NO: 419 - protein, 12 aa, Artificial sequence
  Synthetic sequence: Peptide derivative
GTLDFTVSAQAD

SEQ ID NO: 420 - protein, 13 aa, Artificial sequence
  Synthetic sequence: Peptide derivative
TLDFTVSAQADKL

SEQ ID NO: 421 - protein, 12 aa, Artificial sequence
  Synthetic sequence: Peptide derivative
TLDFTVSAQADK

SEQ ID NO: 422 - protein, 11 aa, Artificial sequence
  Synthetic sequence: Peptide derivative
TLDFTVSAQAD

SEQ ID NO: 423 - protein, 12 aa, Alternaria alternata
DHKWYSCGENSF

SEQ ID NO: 424 - protein, 11 aa, Alternaria alternata
HKWYSCGENSF

SEQ ID NO: 425 - protein, 10 aa, Alternaria alternata
KWYSCGENSF

SEQ ID NO: 426 - protein, 12 aa, Alternaria alternata
WYSCGENSFLDF

SEQ ID NO: 427 - protein, 11 aa, Alternaria alternata
WYSCGENSFLD

SEQ ID NO: 428 - protein, 10 aa, Alternaria alternata
WYSCGENSFL

SEQ ID NO: 429 - protein, 15 aa, Alternaria alternata
DHKWYSCGENSFLDF

SEQ ID NO: 430 - protein, 14 aa, Alternaria alternata
DHKWYSCGENSFLD

SEQ ID NO: 431 - protein, 13 aa, Alternaria alternata
DHKWYSCGENSFL

SEQ ID NO: 432 - protein, 14 aa, Alternaria alternata
HKWYSCGENSFLDF

SEQ ID NO: 433 - protein, 13 aa, Alternaria alternata
HKWYSCGENSFLD

SEQ ID NO: 434 - protein, 12 aa, Alternaria alternata
HKWYSCGENSFL

SEQ ID NO: 435 - protein, 13 aa, Alternaria alternata
KWYSCGENSFLDF

SEQ ID NO: 436 - protein, 12 aa, Alternaria alternata
KWYSCGENSFLD

SEQ ID NO: 437 - protein, 11 aa, Alternaria alternata
KWYSCGENSFL

SEQ ID NO: 438 - protein, 12 aa, Artificial sequence
  Synthetic sequence: Peptide derivative
DHKWYSVGENSF

SEQ ID NO: 439 - protein, 11 aa, Artificial sequence
  Synthetic sequence: Peptide derivative
HKWYSVGENSF

SEQ ID NO: 440 - protein, 10 aa, Artificial sequence
  Synthetic sequence: Peptide derivative
KWYSVGENSF

SEQ ID NO: 441 - protein, 12 aa, Artificial sequence
  Synthetic sequence: Peptide derivative
WYSVGENSFLDF

SEQ ID NO: 442 - protein, 11 aa, Artificial sequence
  Synthetic sequence: Peptide derivative
WYSVGENSFLD

SEQ ID NO: 443 - protein, 10 aa, Artificial sequence
  Synthetic sequence: Peptide derivative
WYSVGENSFL

SEQ ID NO: 444 - protein, 15 aa, Artificial sequence
  Synthetic sequence: Peptide derivative
DHKWYSVGENSFLDF

SEQ ID NO: 445 - protein, 14 aa, Artificial sequence
  Synthetic sequence: Peptide derivative
DHKWYSVGENSFLD

SEQ ID NO: 446 - protein, 13 aa, Artificial sequence
  Synthetic sequence: Peptide derivative
DHKWYSVGENSFL

SEQ ID NO: 447 - protein, 14 aa, Artificial sequence
  Synthetic sequence: Peptide derivative
HKWYSVGENSFLDF

SEQ ID NO: 448 - protein, 13 aa, Artificial sequence
  Synthetic sequence: Peptide derivative
HKWYSVGENSFLD

SEQ ID NO: 449 - protein, 12 aa, Artificial sequence
  Synthetic sequence: Peptide derivative
HKWYSVGENSFL

SEQ ID NO: 450 - protein, 13 aa, Artificial sequence
  Synthetic sequence: Peptide derivative
KWYSVGENSFLDF

SEQ ID NO: 451 - protein, 12 aa, Artificial sequence
  Synthetic sequence: Peptide derivative
KWYSVGENSFLD

SEQ ID NO: 452 - protein, 11 aa, Artificial sequence
  Synthetic sequence: Peptide derivative
KWYSVGENSFL

SEQ ID NO: 453 - protein, 12 aa, Alternaria alternata
KQKVSDDITYVA

SEQ ID NO: 454 - protein, 11 aa, Alternaria alternata
QKVSDDITYVA

SEQ ID NO: 455 - protein, 10 aa, Alternaria alternata
KVSDDITYVA

SEQ ID NO: 456 - protein, 12 aa, Alternaria alternata
VSDDITYVATAT

SEQ ID NO: 457 - protein, 11 aa, Alternaria alternata
VSDDITYVATA

SEQ ID NO: 458 - protein, 10 aa, Alternaria alternata
VSDDITYVAT

SEQ ID NO: 459 - protein, 15 aa, Alternaria alternata
KQKVSDDITYVATAT

SEQ ID NO: 460 - protein, 14 aa, Alternaria alternata
KQKVSDDITYVATA

SEQ ID NO: 461 - protein, 13 aa, Alternaria alternata
KQKVSDDITYVAT

SEQ ID NO: 462 - protein, 14 aa, Alternaria alternata
QKVSDDITYVATAT

SEQ ID NO: 463 - protein, 13 aa, Alternaria alternata
QKVSDDITYVATA

SEQ ID NO: 464 - protein, 12 aa, Alternaria alternata
QKVSDDITYVAT

SEQ ID NO: 465 - protein, 13 aa, Alternaria alternata
KVSDDITYVATAT

SEQ ID NO: 466 - protein, 12 aa, Alternaria alternata
KVSDDITYVATA

SEQ ID NO: 467 - protein, 11 aa, Alternaria alternata
KVSDDITYVAT

SEQ ID NO: 468 - protein, 12 aa, Alternaria alternata
DNRLVNHFTNEF

SEQ ID NO: 469 - protein, 11 aa, Alternaria alternata
NRLVNHFTNEF

SEQ ID NO: 470 - protein, 10 aa, Alternaria alternata
RLVNHFTNEF

SEQ ID NO: 471 - protein, 12 aa, Alternaria alternata
LVNHFTNEFKRK

SEQ ID NO: 472 - protein, 11 aa, Alternaria alternata
LVNHFTNEFKR

SEQ ID NO: 473 - protein, 10 aa, Alternaria alternata
LVNHFTNEFK

SEQ ID NO: 474 - protein, 15 aa, Alternaria alternata
DNRLVNHFTNEFKRK

SEQ ID NO: 475 - protein, 14 aa, Alternaria alternata
DNRLVNHFTNEFKR

SEQ ID NO: 476 - protein, 13 aa, Alternaria alternata
DNRLVNHFTNEFK

SEQ ID NO: 477 - protein, 14 aa, Alternaria alternata
NRLVNHFTNEFKRK

SEQ ID NO: 478 - protein, 13 aa, Alternaria alternata
NRLVNHFTNEFKR

SEQ ID NO: 479 - protein, 12 aa, Alternaria alternata
NRLVNHFTNEFK

SEQ ID NO: 480 - protein, 13 aa, Alternaria alternata
RLVNHFTNEFKRK

SEQ ID NO: 481 - protein, 12 aa, Alternaria alternata
RLVNHFTNEFKR

SEQ ID NO: 482 - protein, 11 aa, Alternaria alternata
RLVNHFTNEFK

SEQ ID NO: 483 - protein, 12 aa, Alternaria alternata
TSEILLLDVAPL

SEQ ID NO: 484 - protein, 11 aa, Alternaria alternata
SEILLLDVAPL

SEQ ID NO: 485 - protein, 10 aa, Alternaria alternata
EILLLDVAPL

SEQ ID NO: 486 - protein, 12 aa, Alternaria alternata
ILLLDVAPLSIG

SEQ ID NO: 487 - protein, 11 aa, Alternaria alternata
ILLLDVAPLSI

SEQ ID NO: 488 - protein, 10 aa, Alternaria alternata
ILLLDVAPLS

SEQ ID NO: 489 - protein, 15 aa, Alternaria alternata
TSEILLLDVAPLSIG

SEQ ID NO: 490 - protein, 14 aa, Alternaria alternata
TSEILLLDVAPLSI

SEQ ID NO: 491 - protein, 13 aa, Alternaria alternata
TSEILLLDVAPLS

SEQ ID NO: 492 - protein, 14 aa, Alternaria alternata
SEILLLDVAPLSIG

SEQ ID NO: 493 - protein, 13 aa, Alternaria alternata
SEILLLDVAPLSI

SEQ ID NO: 494 - protein, 12 aa, Alternaria alternata
SEILLLDVAPLS

SEQ ID NO: 495 - protein, 13 aa, Alternaria alternata
EILLLDVAPLSIG

SEQ ID NO: 496 - protein, 12 aa, Alternaria alternata
EILLLDVAPLSI

SEQ ID NO: 497 - protein, 11 aa, Alternaria alternata
EILLLDVAPLS

SEQ ID NO: 498 - protein, 12 aa, Alternaria alternata
LNVLRIINEPTA

SEQ ID NO: 499 - protein, 11 aa, Alternaria alternata
NVLRIINEPTA

SEQ ID NO: 500 - protein, 10 aa, Alternaria alternata
VLRIINEPTA

SEQ ID NO: 501 - protein, 12 aa, Alternaria alternata
LRIINEPTAAAI

SEQ ID NO: 502 - protein, 11 aa, Alternaria alternata
LRIINEPTAAA

SEQ ID NO: 503 - protein, 10 aa, Alternaria alternata
LRIINEPTAA

SEQ ID NO: 504 - protein, 15 aa, Alternaria alternata
LNVLRIINEPTAAAI

SEQ ID NO: 505 - protein, 14 aa, Alternaria alternata
LNVLRIINEPTAAA

SEQ ID NO: 506 - protein, 13 aa, Alternaria alternata
LNVLRIINEPTAA

SEQ ID NO: 507 - protein, 14 aa, Alternaria alternata
NVLRIINEPTAAAI

SEQ ID NO: 508 - protein, 13 aa, Alternaria alternata
NVLRIINEPTAAA

SEQ ID NO: 509 - protein, 12 aa, Alternaria alternata
NVLRIINEPTAA

SEQ ID NO: 510 - protein, 13 aa, Alternaria alternata
VLRIINEPTAAAI

SEQ ID NO: 511 - protein, 12 aa, Alternaria alternata
VLRIINEPTAAA

SEQ ID NO: 512 - protein, 11 aa, Alternaria alternata
VLRIINEPTAA

SEQ ID NO: 513 - protein, 12 aa, Alternaria alternata
ARALRRLRTACE

SEQ ID NO: 514 - protein, 11 aa, Alternaria alternata
RALRRLRTACE

SEQ ID NO: 515 - protein, 10 aa, Alternaria alternata
ALRRLRTACE

SEQ ID NO: 516 - protein, 12 aa, Alternaria alternata
LRRLRTACERAK

SEQ ID NO: 517 - protein, 11 aa, Alternaria alternata
LRRLRTACERA

SEQ ID NO: 518 - protein, 10 aa, Alternaria alternata
LRRLRTACER

SEQ ID NO: 519 - protein, 15 aa, Alternaria alternata
ARALRRLRTACERAK

SEQ ID NO: 520 - protein, 14 aa, Alternaria alternata
ARALRRLRTACERA

SEQ ID NO: 521 - protein, 13 aa, Alternaria alternata
ARALRRLRTACER

SEQ ID NO: 522 - protein, 14 aa, Alternaria alternata
RALRRLRTACERAK

SEQ ID NO: 523 - protein, 13 aa, Alternaria alternata
RALRRLRTACERA

SEQ ID NO: 524 - protein, 12 aa, Alternaria alternata
RALRRLRTACER

SEQ ID NO: 525 - protein, 13 aa, Alternaria alternata
ALRRLRTACERAK

SEQ ID NO: 526 - protein, 12 aa, Alternaria alternata
ALRRLRTACERA

SEQ ID NO: 527 - protein, 11 aa, Alternaria alternata
ALRRLRTACER

SEQ ID NO: 528 - protein, 12 aa, Alternaria alternata
PDEIQGFPTIKL

SEQ ID NO: 529 - protein, 11 aa, Alternaria alternata
DEIQGFPTIKL

SEQ ID NO: 530 - protein, 10 aa, Alternaria alternata
EIQGFPTIKL

SEQ ID NO: 531 - protein, 12 aa, Alternaria alternata
IQGFPTIKLFPA

SEQ ID NO: 532 - protein, 11 aa, Alternaria alternata
IQGFPTIKLFP

SEQ ID NO: 533 - protein, 10 aa, Alternaria alternata
IQGFPTIKLF

SEQ ID NO: 534 - protein, 15 aa, Alternaria alternata
PDEIQGFPTIKLFPA

SEQ ID NO: 535 - protein, 14 aa, Alternaria alternata
PDEIQGFPTIKLFP

SEQ ID NO: 536 - protein, 13 aa, Alternaria alternata
PDEIQGFPTIKLF

SEQ ID NO: 537 - protein, 14 aa, Alternaria alternata
DEIQGFPTIKLFPA

SEQ ID NO: 538 - protein, 13 aa, Alternaria alternata
DEIQGFPTIKLFP

SEQ ID NO: 539 - protein, 12 aa, Alternaria alternata
DEIQGFPTIKLF

SEQ ID NO: 540 - protein, 13 aa, Alternaria alternata
EIQGFPTIKLFPA

SEQ ID NO: 541 - protein, 12 aa, Alternaria alternata
EIQGFPTIKLFP

SEQ ID NO: 542 - protein, 11 aa, Alternaria alternata
EIQGFPTIKLF

SEQ ID NO: 543 - protein, 12 aa, Alternaria alternata
ADIVVGLRSGQI

SEQ ID NO: 544 - protein, 11 aa, Alternaria alternata
DIVVGLRSGQI

SEQ ID NO: 545 - protein, 10 aa, Alternaria alternata
IVVGLRSGQI

SEQ ID NO: 546 - protein, 12 aa, Alternaria alternata
VVGLRSGQIKTG

SEQ ID NO: 547 - protein, 11 aa, Alternaria alternata
VVGLRSGQIKT

SEQ ID NO: 548 - protein, 10 aa, Alternaria alternata
VVGLRSGQIK

SEQ ID NO: 549 - protein, 15 aa, Alternaria alternata
ADIVVGLRSGQIKTG

SEQ ID NO: 550 - protein, 14 aa, Alternaria alternata
ADIVVGLRSGQIKT

SEQ ID NO: 551 - protein, 13 aa, Alternaria alternata
ADIVVGLRSGQIK

SEQ ID NO: 552 - protein, 14 aa, Alternaria alternata
DIVVGLRSGQIKTG

SEQ ID NO: 553 - protein, 13 aa, Alternaria alternata
DIVVGLRSGQIKT

SEQ ID NO: 554 - protein, 12 aa, Alternaria alternata
DIVVGLRSGQIK

SEQ ID NO: 555 - protein, 13 aa, Alternaria alternata
IVVGLRSGQIKTG

SEQ ID NO: 556 - protein, 12 aa, Alternaria alternata
IVVGLRSGQIKT

SEQ ID NO: 557 - protein, 11 aa, Alternaria alternata
IVVGLRSGQIK

SEQ ID NO: 558 - protein, 12 aa, Alternaria alternata
AGVFVSTGTLGG

SEQ ID NO: 559 - protein, 11 aa, Alternaria alternata
GVFVSTGTLGG

SEQ ID NO: 560 - protein, 10 aa, Alternaria alternata
VFVSTGTLGG

SEQ ID NO: 561 - protein, 12 aa, Alternaria alternata
FVSTGTLGGGQE

SEQ ID NO: 562 - protein, 11 aa, Alternaria alternata
FVSTGTLGGGQ

SEQ ID NO: 563 - protein, 10 aa, Alternaria alternata
FVSTGTLGGG

SEQ ID NO: 564 - protein, 15 aa, Alternaria alternata
AGVFVSTGTLGGGQE

SEQ ID NO: 565 - protein, 14 aa, Alternaria alternata
AGVFVSTGTLGGGQ

SEQ ID NO: 566 - protein, 13 aa, Alternaria alternata
AGVFVSTGTLGGG

SEQ ID NO: 567 - protein, 14 aa, Alternaria alternata
GVFVSTGTLGGGQE

SEQ ID NO: 568 - protein, 13 aa, Alternaria alternata
GVFVSTGTLGGGQ

SEQ ID NO: 569 - protein, 12 aa, Alternaria alternata
GVFVSTGTLGGG

SEQ ID NO: 570 - protein, 13 aa, Alternaria alternata
VFVSTGTLGGGQE

SEQ ID NO: 571 - protein, 12 aa, Alternaria alternata
VFVSTGTLGGGQ

SEQ ID NO: 572 - protein, 11 aa, Alternaria alternata
VFVSTGTLGGG

SEQ ID NO: 573 - protein, 12 aa, Alternaria alternata
KGKVVIVTGASG

SEQ ID NO: 574 - protein, 11 aa, Alternaria alternata
GKVVIVTGASG

SEQ ID NO: 575 - protein, 10 aa, Alternaria alternata
KVVIVTGASG

SEQ ID NO: 576 - protein, 12 aa, Alternaria alternata
VVIVTGASGPTG

SEQ ID NO: 577 - protein, 11 aa, Alternaria alternata
VVIVTGASGPT

SEQ ID NO: 578 - protein, 10 aa, Alternaria alternata
VVIVTGASGP

SEQ ID NO: 579 - protein, 15 aa, Alternaria alternata
KGKVVIVTGASGPTG

SEQ ID NO: 580 - protein, 14 aa, Alternaria alternata
KGKVVIVTGASGPT

SEQ ID NO: 581 - protein, 13 aa, Alternaria alternata
KGKVVIVTGASGP

SEQ ID NO: 582 - protein, 14 aa, Alternaria alternata
GKVVIVTGASGPTG

SEQ ID NO: 583 - protein, 13 aa, Alternaria alternata
GKVVIVTGASGPT

SEQ ID NO: 584 - protein, 12 aa, Alternaria alternata
GKVVIVTGASGP

SEQ ID NO: 585 - protein, 13 aa, Alternaria alternata
KVVIVTGASGPTG

SEQ ID NO: 586 - protein, 12 aa, Alternaria alternata
KVVIVTGASGPT

SEQ ID NO: 587 - protein, 11 aa, Alternaria alternata
KVVIVTGASGP

SEQ ID NO: 588 - protein, 12 aa, Alternaria alternata
TGSLVITSSMSG

SEQ ID NO: 589 - protein, 11 aa, Alternaria alternata
GSLVITSSMSG

SEQ ID NO: 590 - protein, 10 aa, Alternaria alternata
SLVITSSMSG

SEQ ID NO: 591 - protein, 12 aa, Alternaria alternata
LVITSSMSGHIA

SEQ ID NO: 592 - protein, 11 aa, Alternaria alternata
LVITSSMSGHI

SEQ ID NO: 593 - protein, 10 aa, Alternaria alternata
LVITSSMSGH

SEQ ID NO: 594 - protein, 15 aa, Alternaria alternata
TGSLVITSSMSGHIA

SEQ ID NO: 595 - protein, 14 aa, Alternaria alternata
TGSLVITSSMSGHI

SEQ ID NO: 596 - protein, 13 aa, Alternaria alternata
TGSLVITSSMSGH

SEQ ID NO: 597 - protein, 14 aa, Alternaria alternata
GSLVITSSMSGHIA

SEQ ID NO: 598 - protein, 13 aa, Alternaria alternata
GSLVITSSMSGHI

SEQ ID NO: 599 - protein, 12 aa, Alternaria alternata
GSLVITSSMSGH

SEQ ID NO: 600 - protein, 13 aa, Alternaria alternata
SLVITSSMSGHIA

SEQ ID NO: 601 - protein, 12 aa, Alternaria alternata
SLVITSSMSGHI

SEQ ID NO: 602 - protein, 11 aa, Alternaria alternata
SLVITSSMSGH

SEQ ID NO: 603 - protein, 12 aa, Artificial sequence
  Synthetic sequence: Peptide derivative
TGSLVITSSLSG

SEQ ID NO: 604 - protein, 11 aa, Artificial sequence
  Synthetic sequence: Peptide derivative
GSLVITSSLSG

SEQ ID NO: 605 - protein, 10 aa, Artificial sequence
  Synthetic sequence: Peptide derivative
SLVITSSLSG

SEQ ID NO: 606 - protein, 12 aa, Artificial sequence
  Synthetic sequence: Peptide derivative
LVITSSLSGHIA

SEQ ID NO: 607 - protein, 11 aa, Artificial sequence
  Synthetic sequence: Peptide derivative
LVITSSLSGHI

SEQ ID NO: 608 - protein, 10 aa, Artificial sequence
  Synthetic sequence: Peptide derivative
LVITSSLSGH

SEQ ID NO: 609 - protein, 15 aa, Artificial sequence
  Synthetic sequence: Peptide derivative
TGSLVITSSLSGHIA

SEQ ID NO: 610 - protein, 14 aa, Artificial sequence
  Synthetic sequence: Peptide derivative
TGSLVITSSLSGHI

SEQ ID NO: 611 - protein, 13 aa, Artificial sequence
  Synthetic sequence: Peptide derivative
TGSLVITSSLSGH

SEQ ID NO: 612 - protein, 14 aa, Artificial sequence
  Synthetic sequence: Peptide derivative
GSLVITSSLSGHIA

SEQ ID NO: 613 - protein, 13 aa, Artificial sequence
  Synthetic sequence: Peptide derivative
GSLVITSSLSGHI

SEQ ID NO: 614 - protein, 12 aa, Artificial sequence
  Synthetic sequence: Peptide derivative
GSLVITSSLSGH

SEQ ID NO: 615 - protein, 13 aa, Artificial sequence
  Synthetic sequence: Peptide derivative
SLVITSSLSGHIA

SEQ ID NO: 616 - protein, 12 aa, Artificial sequence
  Synthetic sequence: Peptide derivative
SLVITSSLSGHI

SEQ ID NO: 617 - protein, 11 aa, Artificial sequence
  Synthetic sequence: Peptide derivative
SLVITSSLSGH

SEQ ID NO: 618 - protein, 12 aa, Alternaria alternata
VWAIVLLSLCQL

SEQ ID NO: 619 - protein, 11 aa, Alternaria alternata
WAIVLLSLCQL

SEQ ID NO: 620 - protein, 10 aa, Alternaria alternata
AIVLLSLCQL

SEQ ID NO: 621 - protein, 12 aa, Alternaria alternata
IVLLSLCQLTTP

SEQ ID NO: 622 - protein, 11 aa, Alternaria alternata
IVLLSLCQLTT

SEQ ID NO: 623 - protein, 10 aa, Alternaria alternata
IVLLSLCQLT

SEQ ID NO: 624 - protein, 15 aa, Alternaria alternata
VWAIVLLSLCQLTTP

SEQ ID NO: 625 - protein, 14 aa, Alternaria alternata
VWAIVLLSLCQLTT

SEQ ID NO: 626 - protein, 13 aa, Alternaria alternata
VWAIVLLSLCQLT

SEQ ID NO: 627 - protein, 14 aa, Alternaria alternata
WAIVLLSLCQLTTP

SEQ ID NO: 628 - protein, 13 aa, Alternaria alternata
WAIVLLSLCQLTT

SEQ ID NO: 629 - protein, 12 aa, Alternaria alternata
WAIVLLSLCQLT

SEQ ID NO: 630 - protein, 13 aa, Alternaria alternata
AIVLLSLCQLTTP

SEQ ID NO: 631 - protein, 12 aa, Alternaria alternata
AIVLLSLCQLTT

SEQ ID NO: 632 - protein, 11 aa, Alternaria alternata
AIVLLSLCQLT

SEQ ID NO: 633 - protein, 12 aa, Artificial sequence
  Synthetic sequence: Peptide derivative
VWAIVLLSLVQL

SEQ ID NO: 634 - protein, 11 aa, Artificial sequence
  Synthetic sequence: Peptide derivative
WAIVLLSLVQL

SEQ ID NO: 635 - protein, 10 aa, Artificial sequence
  Synthetic sequence: Peptide derivative
AIVLLSLVQL

SEQ ID NO: 636 - protein, 12 aa, Artificial sequence
  Synthetic sequence: Peptide derivative
IVLLSLVQLTTP

SEQ ID NO: 637 - protein, 11 aa, Artificial sequence
  Synthetic sequence: Peptide derivative
IVLLSLVQLTT

SEQ ID NO: 638 - protein, 10 aa, Artificial sequence
  Synthetic sequence: Peptide derivative
IVLLSLVQLT

SEQ ID NO: 639 - protein, 15 aa, Artificial sequence
  Synthetic sequence: Peptide derivative
VWAIVLLSLVQLTTP

SEQ ID NO: 640 - protein, 14 aa, Artificial sequence
  Synthetic sequence: Peptide derivative
VWAIVLLSLVQLTT

SEQ ID NO: 641 - protein, 13 aa, Artificial sequence
  Synthetic sequence: Peptide derivative
VWAIVLLSLVQLT

SEQ ID NO: 642 - protein, 14 aa, Artificial sequence
  Synthetic sequence: Peptide derivative
WAIVLLSLVQLTTP

SEQ ID NO: 643 - protein, 13 aa, Artificial sequence
  Synthetic sequence: Peptide derivative
WAIVLLSLVQLTT

SEQ ID NO: 644 - protein, 12 aa, Artificial sequence
  Synthetic sequence: Peptide derivative
WAIVLLSLVQLT

SEQ ID NO: 645 - protein, 13 aa, Artificial sequence
  Synthetic sequence: Peptide derivative
AIVLLSLVQLTTP

SEQ ID NO: 646 - protein, 12 aa, Artificial sequence
  Synthetic sequence: Peptide derivative
AIVLLSLVQLTT

SEQ ID NO: 647 - protein, 11 aa, Artificial sequence
  Synthetic sequence: Peptide derivative
AIVLLSLVQLT

SEQ ID NO: 648 - protein, 12 aa, Alternaria alternata
VGIFRDDRIEII

SEQ ID NO: 649 - protein, 11 aa, Alternaria alternata
GIFRDDRIEII

SEQ ID NO: 650 - protein, 10 aa, Alternaria alternata
IFRDDRIEII

SEQ ID NO: 651 - protein, 12 aa, Alternaria alternata
FRDDRIEIIAND

SEQ ID NO: 652 - protein, 11 aa, Alternaria alternata
FRDDRIEIIAN

SEQ ID NO: 653 - protein, 10 aa, Alternaria alternata
FRDDRIEIIA

SEQ ID NO: 654 - protein, 15 aa, Alternaria alternata
VGIFRDDRIEIIAND

SEQ ID NO: 655 - protein, 14 aa, Alternaria alternata
VGIFRDDRIEIIAN

SEQ ID NO: 656 - protein, 13 aa, Alternaria alternata
VGIFRDDRIEIIA

SEQ ID NO: 657 - protein, 14 aa, Alternaria alternata
GIFRDDRIEIIAND

SEQ ID NO: 658 - protein, 13 aa, Alternaria alternata
GIFRDDRIEIIAN

SEQ ID NO: 659 - protein, 12 aa, Alternaria alternata
GIFRDDRIEIIA

SEQ ID NO: 660 - protein, 13 aa, Alternaria alternata
IFRDDRIEIIAND

SEQ ID NO: 661 - protein, 12 aa, Alternaria alternata
IFRDDRIEIIAN

SEQ ID NO: 662 - protein, 11 aa, Alternaria alternata
IFRDDRIEIIA

SEQ ID NO: 663-666 - skipped (4 entries)

SEQ ID NO: 667 - protein, 12 aa, Alternaria alternata
KNQVAMNPVNTV

SEQ ID NO: 668 - protein, 11 aa, Alternaria alternata
NQVAMNPVNTV

SEQ ID NO: 669 - protein, 10 aa, Alternaria alternata
QVAMNPVNTV

SEQ ID NO: 670 - protein, 12 aa, Alternaria alternata
VAMNPVNTVFDA

SEQ ID NO: 671 - protein, 11 aa, Alternaria alternata
VAMNPVNTVFD

SEQ ID NO: 672 - protein, 10 aa, Alternaria alternata
VAMNPVNTVF

SEQ ID NO: 673 - protein, 15 aa, Alternaria alternata
KNQVAMNPVNTVFDA

SEQ ID NO: 674 - protein, 14 aa, Alternaria alternata
KNQVAMNPVNTVFD

SEQ ID NO: 675 - protein, 13 aa, Alternaria alternata
KNQVAMNPVNTVF

SEQ ID NO: 676 - protein, 14 aa, Alternaria alternata
NQVAMNPVNTVFDA

SEQ ID NO: 677 - protein, 13 aa, Alternaria alternata
NQVAMNPVNTVFD

SEQ ID NO: 678 - protein, 12 aa, Alternaria alternata
NQVAMNPVNTVF

SEQ ID NO: 679 - protein, 13 aa, Alternaria alternata
QVAMNPVNTVFDA

SEQ ID NO: 680 - protein, 12 aa, Alternaria alternata
QVAMNPVNTVFD

SEQ ID NO: 681 - protein, 11 aa, Alternaria alternata
QVAMNPVNTVF

SEQ ID NO: 682 - protein, 12 aa, Artificial sequence
  Synthetic sequence: Peptide derivative
KNQVALNPVNTV

SEQ ID NO: 683 - protein, 11 aa, Artificial sequence
  Synthetic sequence: Peptide derivative
NQVALNPVNTV

SEQ ID NO: 684 - protein, 10 aa, Artificial sequence
  Synthetic sequence: Peptide derivative
QVALNPVNTV

SEQ ID NO: 685 - protein, 12 aa, Artificial sequence
  Synthetic sequence: Peptide derivative
VALNPVNTVFDA

SEQ ID NO: 686 - protein, 11 aa, Artificial sequence
  Synthetic sequence: Peptide derivative
VALNPVNTVFD

SEQ ID NO: 687 - protein, 10 aa, Artificial sequence
  Synthetic sequence: Peptide derivative
VALNPVNTVF

SEQ ID NO: 688 - protein, 15 aa, Artificial sequence
  Synthetic sequence: Peptide derivative
KNQVALNPVNTVFDA

SEQ ID NO: 689 - protein, 14 aa, Artificial sequence
  Synthetic sequence: Peptide derivative
KNQVALNPVNTVFD

SEQ ID NO: 690 - protein, 13 aa, Artificial sequence
  Synthetic sequence: Peptide derivative
KNQVALNPVNTVF

SEQ ID NO: 691 - protein, 14 aa, Artificial sequence
  Synthetic sequence: Peptide derivative
NQVALNPVNTVFDA

SEQ ID NO: 692 - protein, 13 aa, Artificial sequence
  Synthetic sequence: Peptide derivative
NQVALNPVNTVFD

SEQ ID NO: 693 - protein, 12 aa, Artificial sequence
  Synthetic sequence: Peptide derivative
NQVALNPVNTVF

SEQ ID NO: 694 - protein, 13 aa, Artificial sequence
  Synthetic sequence: Peptide derivative
QVALNPVNTVFDA

SEQ ID NO: 695 - protein, 12 aa, Artificial sequence
  Synthetic sequence: Peptide derivative
QVALNPVNTVFD

SEQ ID NO: 696 - protein, 11 aa, Artificial sequence
  Synthetic sequence: Peptide derivative
QVALNPVNTVF

SEQ ID NO: 697 - protein, 12 aa, Alternaria alternata
LSKLVTIAKVDA

SEQ ID NO: 698 - protein, 11 aa, Alternaria alternata
SKLVTIAKVDA

SEQ ID NO: 699 - protein, 10 aa, Alternaria alternata
KLVTIAKVDA

SEQ ID NO: 700 - protein, 12 aa, Alternaria alternata
LVTIAKVDATLN

SEQ ID NO: 701 - protein, 11 aa, Alternaria alternata
LVTIAKVDATL

SEQ ID NO: 702 - protein, 10 aa, Alternaria alternata
LVTIAKVDAT

SEQ ID NO: 703 - protein, 15 aa, Alternaria alternata
LSKLVTIAKVDATLN

SEQ ID NO: 704 - protein, 14 aa, Alternaria alternata
LSKLVTIAKVDATL

SEQ ID NO: 705 - protein, 13 aa, Alternaria alternata
LSKLVTIAKVDAT

SEQ ID NO: 706 - protein, 14 aa, Alternaria alternata
SKLVTIAKVDATLN

SEQ ID NO: 707 - protein, 13 aa, Alternaria alternata
SKLVTIAKVDATL

SEQ ID NO: 708 - protein, 12 aa, Alternaria alternata
SKLVTIAKVDAT

SEQ ID NO: 709 - protein, 13 aa, Alternaria alternata
KLVTIAKVDATLN

SEQ ID NO: 710 - protein, 12 aa, Alternaria alternata
KLVTIAKVDATL

SEQ ID NO: 711 - protein, 11 aa, Alternaria alternata
KLVTIAKVDAT

SEQ ID NO: 712 - protein, 15 aa, Alternaria alternata
MKHLAAYLLLGLGGN

SEQ ID NO: 713 - protein, 14 aa, Alternaria alternata
MKHLAAYLLLGLGG

SEQ ID NO: 714 - protein, 13 aa, Alternaria alternata
MKHLAAYLLLGLG

SEQ ID NO: 715 - protein, 12 aa, Alternaria alternata
MKHLAAYLLLGL

SEQ ID NO: 716 - protein, 11 aa, Alternaria alternata
MKHLAAYLLLG

SEQ ID NO: 717 - protein, 10 aa, Alternaria alternata
MKHLAAYLLL

SEQ ID NO: 718 - protein, 15 aa, Artificial sequence
  Synthetic sequence: Peptide derivative
LKHLAAYLLLGLGGN

SEQ ID NO: 719 - protein, 14 aa, Artificial sequence
  Synthetic sequence: Peptide derivative
LKHLAAYLLLGLGG

SEQ ID NO: 720 - protein, 13 aa, Artificial sequence
  Synthetic sequence: Peptide derivative
LKHLAAYLLLGLG

SEQ ID NO: 721 - protein, 12 aa, Artificial sequence
  Synthetic sequence: Peptide derivative
LKHLAAYLLLGL

SEQ ID NO: 722 - protein, 11 aa, Artificial sequence
  Synthetic sequence: Peptide derivative
LKHLAAYLLLG

SEQ ID NO: 723 - protein, 10 aa, Artificial sequence
  Synthetic sequence: Peptide derivative
LKHLAAYLLL

SEQ ID NO: 724 - protein, 12 aa, Alternaria alternata
AEVYQKLKALAK

SEQ ID NO: 725 - protein, 11 aa, Alternaria alternata
EVYQKLKALAK

SEQ ID NO: 726 - protein, 10 aa, Alternaria alternata
VYQKLKALAK

SEQ ID NO: 727 - protein, 12 aa, Alternaria alternata
YQKLKALAKKTY

SEQ ID NO: 728 - protein, 11 aa, Alternaria alternata
YQKLKALAKKT

SEQ ID NO: 729 - protein, 10 aa, Alternaria alternata
YQKLKALAKK

SEQ ID NO: 730 - protein, 15 aa, Alternaria alternata
AEVYQKLKALAKKTY

SEQ ID NO: 731 - protein, 14 aa, Alternaria alternata
AEVYQKLKALAKKT

SEQ ID NO: 732 - protein, 13 aa, Alternaria alternata
AEVYQKLKALAKK

SEQ ID NO: 733 - protein, 14 aa, Alternaria alternata
EVYQKLKALAKKTY

SEQ ID NO: 734 - protein, 13 aa, Alternaria alternata
EVYQKLKALAKKT

SEQ ID NO: 735 - protein, 12 aa, Alternaria alternata
EVYQKLKALAKK

SEQ ID NO: 736 - protein, 13 aa, Alternaria alternata
VYQKLKALAKKTY

SEQ ID NO: 737 - protein, 12 aa, Alternaria alternata
VYQKLKALAKKT

SEQ ID NO: 738 - protein, 11 aa, Alternaria alternata
VYQKLKALAKK

SEQ ID NO: 739 - protein, 12 aa, Alternaria alternata
AIELKSCNALLL

SEQ ID NO: 740 - protein, 11 aa, Alternaria alternata
IELKSCNALLL

SEQ ID NO: 741 - protein, 10 aa, Alternaria alternata
ELKSCNALLL

SEQ ID NO: 742 - protein, 12 aa, Alternaria alternata
LKSCNALLLKVN

SEQ ID NO: 743 - protein, 11 aa, Alternaria alternata
LKSCNALLLKV

SEQ ID NO: 744 - protein, 10 aa, Alternaria alternata
LKSCNALLLK

SEQ ID NO: 745 - protein, 15 aa, Alternaria alternata
AIELKSCNALLLKVN

SEQ ID NO: 746 - protein, 14 aa, Alternaria alternata
AIELKSCNALLLKV

SEQ ID NO: 747 - protein, 13 aa, Alternaria alternata
AIELKSCNALLLK

SEQ ID NO: 748 - protein, 14 aa, Alternaria alternata
IELKSCNALLLKVN

SEQ ID NO: 749 - protein, 13 aa, Alternaria alternata
IELKSCNALLLKV

SEQ ID NO: 750 - protein, 12 aa, Alternaria alternata
IELKSCNALLLK

SEQ ID NO: 751 - protein, 13 aa, Alternaria alternata
ELKSCNALLLKVN

SEQ ID NO: 752 - protein, 12 aa, Alternaria alternata
ELKSCNALLLKV

SEQ ID NO: 753 - protein, 11 aa, Alternaria alternata
ELKSCNALLLK

SEQ ID NO: 754 - protein, 12 aa, Artificial sequence
  Synthetic sequence: Peptide derivative
AIELKSVNALLL

SEQ ID NO: 755 - protein, 11 aa, Artificial sequence
  Synthetic sequence: Peptide derivative
IELKSVNALLL

SEQ ID NO: 756 - protein, 10 aa, Artificial sequence
  Synthetic sequence: Peptide derivative
ELKSVNALLL

SEQ ID NO: 757 - protein, 12 aa, Artificial sequence
  Synthetic sequence: Peptide derivative
LKSVNALLLKVN

SEQ ID NO: 758 - protein, 11 aa, Artificial sequence
  Synthetic sequence: Peptide derivative
LKSVNALLLKV

SEQ ID NO: 759 - protein, 10 aa, Artificial sequence
  Synthetic sequence: Peptide derivative
LKSVNALLLK

SEQ ID NO: 760 - protein, 15 aa, Artificial sequence
  Synthetic sequence: Peptide derivative
AIELKSVNALLLKVN

SEQ ID NO: 761 - protein, 14 aa, Artificial sequence
  Synthetic sequence: Peptide derivative
AIELKSVNALLLKV

SEQ ID NO: 762 - protein, 13 aa, Artificial sequence
  Synthetic sequence: Peptide derivative
AIELKSVNALLLK

SEQ ID NO: 763 - protein, 14 aa, Artificial sequence
  Synthetic sequence: Peptide derivative
IELKSVNALLLKVN

SEQ ID NO: 764 - protein, 13 aa, Artificial sequence
  Synthetic sequence: Peptide derivative
IELKSVNALLLKV

SEQ ID NO: 765 - protein, 12 aa, Artificial sequence
  Synthetic sequence: Peptide derivative
IELKSVNALLLK

SEQ ID NO: 766 - protein, 13 aa, Artificial sequence
  Synthetic sequence: Peptide derivative
ELKSVNALLLKVN

SEQ ID NO: 767 - protein, 12 aa, Artificial sequence
  Synthetic sequence: Peptide derivative
ELKSVNALLLKV

SEQ ID NO: 768 - protein, 11 aa, Artificial sequence
  Synthetic sequence: Peptide derivative
ELKSVNALLLK

SEQ ID NO: 769 - protein, 12 aa, Alternaria alternata
GAGWGVMVSHRS

SEQ ID NO: 770 - protein, 11 aa, Alternaria alternata
AGWGVMVSHRS

SEQ ID NO: 771 - protein, 10 aa, Alternaria alternata
GWGVMVSHRS

SEQ ID NO: 772 - protein, 12 aa, Alternaria alternata
WGVMVSHRSGET

SEQ ID NO: 773 - protein, 11 aa, Alternaria alternata
WGVMVSHRSGE

SEQ ID NO: 774 - protein, 10 aa, Alternaria alternata
WGVMVSHRSG

SEQ ID NO: 775 - protein, 15 aa, Alternaria alternata
GAGWGVMVSHRSGET

SEQ ID NO: 776 - protein, 14 aa, Alternaria alternata
GAGWGVMVSHRSGE

SEQ ID NO: 777 - protein, 13 aa, Alternaria alternata
GAGWGVMVSHRSG

SEQ ID NO: 778 - protein, 14 aa, Alternaria alternata
AGWGVMVSHRSGET

SEQ ID NO: 779 - protein, 13 aa, Alternaria alternata
AGWGVMVSHRSGE

SEQ ID NO: 780 - protein, 12 aa, Alternaria alternata
AGWGVMVSHRSG

SEQ ID NO: 781 - protein, 13 aa, Alternaria alternata
GWGVMVSHRSGET

SEQ ID NO: 782 - protein, 12 aa, Alternaria alternata
GWGVMVSHRSGE

SEQ ID NO: 783 - protein, 11 aa, Alternaria alternata
GWGVMVSHRSG

SEQ ID NO: 784 - protein, 12 aa, Artificial sequence
  Synthetic sequence: Peptide derivative
GAGWGVLVSHRS

SEQ ID NO: 785 - protein, 11 aa, Artificial sequence
  Synthetic sequence: Peptide derivative
AGWGVLVSHRS

SEQ ID NO: 786 - protein, 10 aa, Artificial sequence
  Synthetic sequence: Peptide derivative
GWGVLVSHRS

SEQ ID NO: 787 - protein, 12 aa, Artificial sequence
  Synthetic sequence: Peptide derivative
WGVLVSHRSGET

SEQ ID NO: 788 - protein, 11 aa, Artificial sequence
  Synthetic sequence: Peptide derivative
WGVLVSHRSGE

SEQ ID NO: 789 - protein, 10 aa, Artificial sequence
  Synthetic sequence: Peptide derivative
WGVLVSHRSG

SEQ ID NO: 790 - protein, 15 aa, Artificial sequence
  Synthetic sequence: Peptide derivative
GAGWGVLVSHRSGET

SEQ ID NO: 791 - protein, 14 aa, Artificial sequence
  Synthetic sequence: Peptide derivative
GAGWGVLVSHRSGE

SEQ ID NO: 792 - protein, 13 aa, Artificial sequence
  Synthetic sequence: Peptide derivative
GAGWGVLVSHRSG

SEQ ID NO: 793 - protein, 14 aa, Artificial sequence
  Synthetic sequence: Peptide derivative
AGWGVLVSHRSGET

SEQ ID NO: 794 - protein, 13 aa, Artificial sequence
  Synthetic sequence: Peptide derivative
AGWGVLVSHRSGE

SEQ ID NO: 795 - protein, 12 aa, Artificial sequence
  Synthetic sequence: Peptide derivative
AGWGVLVSHRSG

SEQ ID NO: 796 - protein, 13 aa, Artificial sequence
  Synthetic sequence: Peptide derivative
GWGVLVSHRSGET

SEQ ID NO: 797 - protein, 12 aa, Artificial sequence
  Synthetic sequence: Peptide derivative
GWGVLVSHRSGE

SEQ ID NO: 798 - protein, 11 aa, Artificial sequence
  Synthetic sequence: Peptide derivative
GWGVLVSHRSG

SEQ ID NO: 799 - protein, 12 aa, Alternaria alternata
VPLGYKTAFSML

SEQ ID NO: 800 - protein, 11 aa, Alternaria alternata
PLGYKTAFSML

SEQ ID NO: 801 - protein, 10 aa, Alternaria alternata
LGYKTAFSML

SEQ ID NO: 802 - protein, 12 aa, Alternaria alternata
GYKTAFSMLANL

SEQ ID NO: 803 - protein, 11 aa, Alternaria alternata
GYKTAFSMLAN

SEQ ID NO: 804 - protein, 10 aa, Alternaria alternata
GYKTAFSMLA

SEQ ID NO: 805 - protein, 15 aa, Alternaria alternata
VPLGYKTAFSMLANL

SEQ ID NO: 806 - protein, 14 aa, Alternaria alternata
VPLGYKTAFSMLAN

SEQ ID NO: 807 - protein, 13 aa, Alternaria alternata
VPLGYKTAFSMLA

SEQ ID NO: 808 - protein, 14 aa, Alternaria alternata
PLGYKTAFSMLANL

SEQ ID NO: 809 - protein, 13 aa, Alternaria alternata
PLGYKTAFSMLAN

SEQ ID NO: 810 - protein, 12 aa, Alternaria alternata
PLGYKTAFSMLA

SEQ ID NO: 811 - protein, 13 aa, Alternaria alternata
LGYKTAFSMLANL

SEQ ID NO: 812 - protein, 12 aa, Alternaria alternata
LGYKTAFSMLAN

SEQ ID NO: 813 - protein, 11 aa, Alternaria alternata
LGYKTAFSMLA

SEQ ID NO: 814 - protein, 12 aa, Artificial sequence
  Synthetic sequence: Peptide derivative
VPLGYKTAFSLL

SEQ ID NO: 815 - protein, 11 aa, Artificial sequence
  Synthetic sequence: Peptide derivative
PLGYKTAFSLL

SEQ ID NO: 816 - protein, 10 aa, Artificial sequence
  Synthetic sequence: Peptide derivative
LGYKTAFSLL

SEQ ID NO: 817 - protein, 12 aa, Artificial sequence
  Synthetic sequence: Peptide derivative
GYKTAFSLLANL

SEQ ID NO: 818 - protein, 11 aa, Artificial sequence
  Synthetic sequence: Peptide derivative
GYKTAFSLLAN

SEQ ID NO: 819 - protein, 10 aa, Artificial sequence
  Synthetic sequence: Peptide derivative
GYKTAFSLLA

SEQ ID NO: 820 - protein, 15 aa, Artificial sequence
  Synthetic sequence: Peptide derivative
VPLGYKTAFSLLANL

SEQ ID NO: 821 - protein, 14 aa, Artificial sequence
  Synthetic sequence: Peptide derivative
VPLGYKTAFSLLAN

SEQ ID NO: 822 - protein, 13 aa, Artificial sequence
  Synthetic sequence: Peptide derivative
VPLGYKTAFSLLA

SEQ ID NO: 823 - protein, 14 aa, Artificial sequence
  Synthetic sequence: Peptide derivative
PLGYKTAFSLLANL

SEQ ID NO: 824 - protein, 13 aa, Artificial sequence
  Synthetic sequence: Peptide derivative
PLGYKTAFSLLAN

SEQ ID NO: 825 - protein, 12 aa, Artificial sequence
  Synthetic sequence: Peptide derivative
PLGYKTAFSLLA

SEQ ID NO: 826 - protein, 13 aa, Artificial sequence
  Synthetic sequence: Peptide derivative
LGYKTAFSLLANL

SEQ ID NO: 827 - protein, 12 aa, Artificial sequence
  Synthetic sequence: Peptide derivative
LGYKTAFSLLAN

SEQ ID NO: 828 - protein, 11 aa, Artificial sequence
  Synthetic sequence: Peptide derivative
LGYKTAFSLLA

SEQ ID NO: 829 - protein, 12 aa, Alternaria alternata
LSDFVPQDIQKL

SEQ ID NO: 830 - protein, 11 aa, Alternaria alternata
SDFVPQDIQKL

SEQ ID NO: 831 - protein, 10 aa, Alternaria alternata
DFVPQDIQKL

SEQ ID NO: 832 - protein, 12 aa, Alternaria alternata
FVPQDIQKLWHS

SEQ ID NO: 833 - protein, 11 aa, Alternaria alternata
FVPQDIQKLWH

SEQ ID NO: 834 - protein, 10 aa, Alternaria alternata
FVPQDIQKLW

SEQ ID NO: 835 - protein, 15 aa, Alternaria alternata
LSDFVPQDIQKLWHS

SEQ ID NO: 836 - protein, 14 aa, Alternaria alternata
LSDFVPQDIQKLWH

SEQ ID NO: 837 - protein, 13 aa, Alternaria alternata
LSDFVPQDIQKLW

SEQ ID NO: 838 - protein, 14 aa, Alternaria alternata
SDFVPQDIQKLWHS

SEQ ID NO: 839 - protein, 13 aa, Alternaria alternata
SDFVPQDIQKLWH

SEQ ID NO: 840 - protein, 12 aa, Alternaria alternata
SDFVPQDIQKLW

SEQ ID NO: 841 - protein, 13 aa, Alternaria alternata
DFVPQDIQKLWHS

SEQ ID NO: 842 - protein, 12 aa, Alternaria alternata
DFVPQDIQKLWH

SEQ ID NO: 843 - protein, 11 aa, Alternaria alternata
DFVPQDIQKLW

SEQ ID NO: 844 - protein, 12 aa, Alternaria alternata
KGAYVYFASDAS

SEQ ID NO: 845 - protein, 11 aa, Alternaria alternata
GAYVYFASDAS

SEQ ID NO: 846 - protein, 10 aa, Alternaria alternata
AYVYFASDAS

SEQ ID NO: 847 - protein, 12 aa, Artificial sequence
  Synthetic sequence: Peptide derivative
YVYFASDASSYV

SEQ ID NO: 848 - protein, 11 aa, Alternaria alternata
YVYFASDASSY

SEQ ID NO: 849 - protein, 10 aa, Alternaria alternata
YVYFASDASS

SEQ ID NO: 850 - protein, 15 aa, Artificial sequence
  Synthetic sequence: Peptide derivative
KGAYVYFASDASSYV

SEQ ID NO: 851 - protein, 14 aa, Alternaria alternata
KGAYVYFASDASSY

SEQ ID NO: 852 - protein, 13 aa, Alternaria alternata
KGAYVYFASDASS

SEQ ID NO: 853 - protein, 14 aa, Artificial sequence
  Synthetic sequence: Peptide derivative
GAYVYFASDASSYV

SEQ ID NO: 854 - protein, 13 aa, Alternaria alternata
GAYVYFASDASSY

SEQ ID NO: 855 - protein, 12 aa, Alternaria alternata
GAYVYFASDASS

SEQ ID NO: 856 - protein, 13 aa, Artificial sequence
  Synthetic sequence: Peptide derivative
AYVYFASDASSYV

SEQ ID NO: 857 - protein, 12 aa, Alternaria alternata
AYVYFASDASSY

SEQ ID NO: 858 - protein, 11 aa, Alternaria alternata
AYVYFASDASS

SEQ ID NO: 859 - protein, 12 aa, Alternaria alternata
YVYFASDASSYC

SEQ ID NO: 860 - protein, 15 aa, Alternaria alternata
KGAYVYFASDASSYC

SEQ ID NO: 861 - protein, 14 aa, Alternaria alternata
GAYVYFASDASSYC

SEQ ID NO: 862 - protein, 13 aa, Alternaria alternata
AYVYFASDASSYC

SEQ ID NO: 863 - protein, 12 aa, Alternaria alternata
DKGYFIEPTIFS

SEQ ID NO: 864 - protein, 11 aa, Alternaria alternata
KGYFIEPTIFS

SEQ ID NO: 865 - protein, 10 aa, Alternaria alternata
GYFIEPTIFS

SEQ ID NO: 866 - protein, 12 aa, Alternaria alternata
YFIEPTIFSNVT

SEQ ID NO: 867 - protein, 11 aa, Alternaria alternata
YFIEPTIFSNV

SEQ ID NO: 868 - protein, 10 aa, Alternaria alternata
YFIEPTIFSN

SEQ ID NO: 869 - protein, 15 aa, Alternaria alternata
DKGYFIEPTIFSNVT

SEQ ID NO: 870 - protein, 14 aa, Alternaria alternata
DKGYFIEPTIFSNV

SEQ ID NO: 871 - protein, 13 aa, Alternaria alternata
DKGYFIEPTIFSN

SEQ ID NO: 872 - protein, 14 aa, Alternaria alternata
KGYFIEPTIFSNVT

SEQ ID NO: 873 - protein, 13 aa, Alternaria alternata
KGYFIEPTIFSNV

SEQ ID NO: 874 - protein, 12 aa, Alternaria alternata
KGYFIEPTIFSN

SEQ ID NO: 875 - protein, 13 aa, Alternaria alternata
GYFIEPTIFSNVT

SEQ ID NO: 876 - protein, 12 aa, Alternaria alternata
GYFIEPTIFSNV

SEQ ID NO: 877 - protein, 11 aa, Alternaria alternata
GYFIEPTIFSN

SEQ ID NO: 878 - protein, 12 aa, Alternaria alternata
LDNYIQTKTVSI

SEQ ID NO: 879 - protein, 11 aa, Alternaria alternata
DNYIQTKTVSI

SEQ ID NO: 880 - protein, 10 aa, Alternaria alternata
NYIQTKTVSI

SEQ ID NO: 881 - protein, 12 aa, Alternaria alternata
YIQTKTVSIRLG

SEQ ID NO: 882 - protein, 11 aa, Alternaria alternata
YIQTKTVSIRL

SEQ ID NO: 883 - protein, 10 aa, Alternaria alternata
YIQTKTVSIR

SEQ ID NO: 884 - protein, 15 aa, Alternaria alternata
LDNYIQTKTVSIRLG

SEQ ID NO: 885 - protein, 14 aa, Alternaria alternata
LDNYIQTKTVSIRL

SEQ ID NO: 886 - protein, 13 aa, Alternaria alternata
LDNYIQTKTVSIR

SEQ ID NO: 887 - protein, 14 aa, Alternaria alternata
DNYIQTKTVSIRLG

SEQ ID NO: 888 - protein, 13 aa, Alternaria alternata
DNYIQTKTVSIRL

SEQ ID NO: 889 - protein, 12 aa, Alternaria alternata
DNYIQTKTVSIR

SEQ ID NO: 890 - protein, 13 aa, Alternaria alternata
NYIQTKTVSIRLG

SEQ ID NO: 891 - protein, 12 aa, Alternaria alternata
NYIQTKTVSIRL

SEQ ID NO: 892 - protein, 11 aa, Alternaria alternata
NYIQTKTVSIR

SEQ ID NO: 893 - protein, 15 aa, Alternaria alternata
QTKTVSIRLGDVLFG

SEQ ID NO: 894 - protein, 14 aa, Alternaria alternata
TKTVSIRLGDVLFG

SEQ ID NO: 895 - protein, 13 aa, Alternaria alternata
KTVSIRLGDVLFG

SEQ ID NO: 896 - protein, 12 aa, Alternaria alternata
TVSIRLGDVLFG

SEQ ID NO: 897 - protein, 11 aa, Alternaria alternata
VSIRLGDVLFG

SEQ ID NO: 898 - protein, 10 aa, Alternaria alternata
SIRLGDVLFG

SEQ ID NO: 899 - protein, 12 aa, Alternaria alternata
GTVWVNSYNTLH

SEQ ID NO: 900 - protein, 11 aa, Alternaria alternata
TVWVNSYNTLH

SEQ ID NO: 901 - protein, 10 aa, Alternaria alternata
VWVNSYNTLH

SEQ ID NO: 902 - protein, 12 aa, Alternaria alternata
WVNSYNTLHWQL

SEQ ID NO: 903 - protein, 11 aa, Alternaria alternata
WVNSYNTLHWQ

SEQ ID NO: 904 - protein, 10 aa, Alternaria alternata
WVNSYNTLHW

SEQ ID NO: 905 - protein, 15 aa, Alternaria alternata
GTVWVNSYNTLHWQL

SEQ ID NO: 906 - protein, 14 aa, Alternaria alternata
GTVWVNSYNTLHWQ

SEQ ID NO: 907 - protein, 13 aa, Alternaria alternata
GTVWVNSYNTLHW

SEQ ID NO: 908 - protein, 14 aa, Alternaria alternata
TVWVNSYNTLHWQL

SEQ ID NO: 909 - protein, 13 aa, Alternaria alternata
TVWVNSYNTLHWQ

SEQ ID NO: 910 - protein, 12 aa, Alternaria alternata
TVWVNSYNTLHW

SEQ ID NO: 911 - protein, 13 aa, Alternaria alternata
VWVNSYNTLHWQL

SEQ ID NO: 912 - protein, 12 aa, Alternaria alternata
VWVNSYNTLHWQ

SEQ ID NO: 913 - protein, 11 aa, Alternaria alternata
VWVNSYNTLHW

SEQ ID NO: 914 - protein, 9 aa, Alternaria alternata
VATATLPNY

SEQ ID NO: 915 - protein, 9 aa, Alternaria alternata
VYQKLKALA

SEQ ID NO: 916 - protein, 9 aa, Alternaria alternata
KLKALAKKT

SEQ ID NO: 917 - protein, 9 aa, Alternaria alternata
LKALAKKTY

SEQ ID NO: 918 - protein, 9 aa, Alternaria alternata
FGAGWGVMV

SEQ ID NO: 919 - protein, 9 aa, Alternaria alternata
GVMVSHRSG

SEQ ID NO: 920 - protein, 9 aa, Alternaria alternata
VMVSHRSGE

SEQ ID NO: 921 - protein, 9 aa, Alternaria alternata
MVSHRSGET

SEQ ID NO: 922 - protein, 9 aa, Alternaria alternata
YVWKISEFY

SEQ ID NO: 923 - protein, 9 aa, Alternaria alternata
LLLKQKVSD

SEQ ID NO: 924 - protein, 9 aa, Alternaria alternata
LLKQKVSDD

SEQ ID NO: 925 - protein, 9 aa, Alternaria alternata
VVLVAYFAA

SEQ ID NO: 926 - protein, 9 aa, Alternaria alternata
VVGRQILKS

SEQ ID NO: 927 - protein, 9 aa, Alternaria alternata
VVGRQIMKS

SEQ ID NO: 928 - protein, 15 aa, Alternaria alternata
EGDYVWKISEFYGRK

SEQ ID NO: 929 - protein, 15 aa, Alternaria alternata
RSGLLLKQKVSDDIT

SEQ ID NO: 930 - protein, 15 aa, Alternaria alternata
SGLLLKQKVSDDITY

SEQ ID NO: 931 - protein, 15 aa, Alternaria alternata
ADKVVLVAYFAADDK

SEQ ID NO: 932 - protein, 15 aa, Artificial sequence
  Synthetic sequence: Peptide derivative
GSTVVGRQILKSAAG

SEQ ID NO: 933 - protein, 9 aa, Dermatophagoides pteronyssinus
LESVKYVQS

SEQ ID NO: 934 - protein, 9 aa, Dermatophagoides pteronyssinus
ESVKYVQSN

SEQ ID NO: 935 - protein, 9 aa, Dermatophagoides pteronyssinus
SVKYVQSNG

SEQ ID NO: 936 - protein, 9 aa, Dermatophagoides pteronyssinus
VKYVQSNGG

SEQ ID NO: 937 - protein, 9 aa, Dermatophagoides pteronyssinus
KYVQSNGGA

SEQ ID NO: 938 - protein, 9 aa, Dermatophagoides pteronyssinus
YVQSNGGAI

SEQ ID NO: 939 - protein, 9 aa, Dermatophagoides pteronyssinus
VQSNGGAIN

SEQ ID NO: 940 - protein, 9 aa, Dermatophagoides pteronyssinus
QSNGGAINH

SEQ ID NO: 941 - protein, 9 aa, Dermatophagoides pteronyssinus
LDEFKNRFL

SEQ ID NO: 942 - protein, 9 aa, Dermatophagoides pteronyssinus
DEFKNRFLM

SEQ ID NO: 943 - protein, 9 aa, Dermatophagoides pteronyssinus
EFKNRFLMS

SEQ ID NO: 944 - protein, 9 aa, Dermatophagoides pteronyssinus
FKNRFLMSA

SEQ ID NO: 945 - protein, 9 aa, Dermatophagoides pteronyssinus
KNRFLMSAE

SEQ ID NO: 946 - protein, 9 aa, Dermatophagoides pteronyssinus
NRFLMSAEA

SEQ ID NO: 947 - protein, 9 aa, Dermatophagoides pteronyssinus
RFLMSAEAF

SEQ ID NO: 948 - protein, 9 aa, Dermatophagoides pteronyssinus
FLMSAEAFE

SEQ ID NO: 949 - protein, 9 aa, Dermatophagoides pteronyssinus
LRQMRTVTP

SEQ ID NO: 950 - protein, 9 aa, Dermatophagoides pteronyssinus
RQMRTVTPI

SEQ ID NO: 951 - protein, 9 aa, Dermatophagoides pteronyssinus
QMRTVTPIR

SEQ ID NO: 952 - protein, 9 aa, Dermatophagoides pteronyssinus
MRTVTPIRM

SEQ ID NO: 953 - protein, 9 aa, Dermatophagoides pteronyssinus
RTVTPIRMQ

SEQ ID NO: 954 - protein, 9 aa, Dermatophagoides pteronyssinus
TVTPIRMQG

SEQ ID NO: 955 - protein, 9 aa, Dermatophagoides pteronyssinus
VTPIRMQGG

SEQ ID NO: 956 - protein, 9 aa, Dermatophagoides pteronyssinus
TPIRMQGGC

SEQ ID NO: 957 - protein, 9 aa, Dermatophagoides pteronyssinus
PIRMQGGCG

SEQ ID NO: 958 - protein, 9 aa, Dermatophagoides pteronyssinus
IRMQGGCGS

SEQ ID NO: 959 - protein, 9 aa, Dermatophagoides pteronyssinus
RMQGGCGSC

SEQ ID NO: 960 - protein, 9 aa, Dermatophagoides pteronyssinus
AYLAYRNQS

SEQ ID NO: 961 - protein, 9 aa, Dermatophagoides pteronyssinus
YLAYRNQSL

SEQ ID NO: 962 - protein, 9 aa, Dermatophagoides pteronyssinus
LAYRNQSLD

SEQ ID NO: 963 - protein, 9 aa, Dermatophagoides pteronyssinus
AYRNQSLDL

SEQ ID NO: 964 - protein, 9 aa, Dermatophagoides pteronyssinus
YRNQSLDLA

SEQ ID NO: 965 - protein, 9 aa, Dermatophagoides pteronyssinus
RNQSLDLAE

SEQ ID NO: 966 - protein, 9 aa, Dermatophagoides pteronyssinus
SYYRYVARE

SEQ ID NO: 967 - protein, 9 aa, Dermatophagoides pteronyssinus
YYRYVAREQ

SEQ ID NO: 968 - protein, 9 aa, Dermatophagoides pteronyssinus
YRYVAREQS

SEQ ID NO: 969 - protein, 9 aa, Dermatophagoides pteronyssinus
RYVAREQSC

SEQ ID NO: 970 - protein, 9 aa, Dermatophagoides pteronyssinus
YVAREQSCR

SEQ ID NO: 971 - protein, 9 aa, Dermatophagoides pteronyssinus
VAREQSCRR

SEQ ID NO: 972 - protein, 9 aa, Dermatophagoides pteronyssinus
AREQSCRRP

SEQ ID NO: 973 - protein, 9 aa, Dermatophagoides pteronyssinus
REQSCRRPN

SEQ ID NO: 974 - protein, 9 aa, Dermatophagoides pteronyssinus
EQSCRRPNA

SEQ ID NO: 975 - protein, 9 aa, Dermatophagoides pteronyssinus
QSCRRPNAQ

SEQ ID NO: 976 - protein, 9 aa, Dermatophagoides pteronyssinus
GYGYFAANI

SEQ ID NO: 977 - protein, 9 aa, Dermatophagoides pteronyssinus
YGYFAANID

SEQ ID NO: 978 - protein, 9 aa, Dermatophagoides pteronyssinus
GYFAANIDL

SEQ ID NO: 979 - protein, 9 aa, Dermatophagoides pteronyssinus
YFAANIDLM

SEQ ID NO: 980 - protein, 9 aa, Dermatophagoides pteronyssinus
FAANIDLMM

SEQ ID NO: 981 - protein, 9 aa, Dermatophagoides pteronyssinus
AANIDLMMI

SEQ ID NO: 982 - protein, 9 aa, Dermatophagoides pteronyssinus
ANIDLMMIE

SEQ ID NO: 983 - protein, 9 aa, Betula pendula
PAARMFKAF

SEQ ID NO: 984 - protein, 9 aa, Betula pendula
AARMFKAFI

SEQ ID NO: 985 - protein, 9 aa, Betula pendula
ARMFKAFIL

SEQ ID NO: 986 - protein, 9 aa, Betula pendula
RMFKAFILD

SEQ ID NO: 987 - protein, 9 aa, Betula pendula
VFNYEIGAT

SEQ ID NO: 988 - protein, 9 aa, Betula pendula
FNYEIGATS

SEQ ID NO: 989 - protein, 9 aa, Betula pendula
NYEIGATSV

SEQ ID NO: 990 - protein, 9 aa, Betula pendula
YEIGATSVI

SEQ ID NO: 991 - protein, 9 aa, Betula pendula
EIGATSVIP

SEQ ID NO: 992 - protein, 9 aa, Betula pendula
IGATSVIPA

SEQ ID NO: 993 - protein, 9 aa, Betula pendula
GATSVIPAA

SEQ ID NO: 994 - protein, 9 aa, Betula pendula
SPFKYVKER

SEQ ID NO: 995 - protein, 9 aa, Betula pendula
PFKYVKERV

SEQ ID NO: 996 - protein, 9 aa, Betula pendula
FKYVKERVD

SEQ ID NO: 997 - protein, 9 aa, Betula pendula
KYVKERVDE

SEQ ID NO: 998 - protein, 9 aa, Betula pendula
YVKERVDEV

SEQ ID NO: 999 - protein, 9 aa, Betula pendula
VKERVDEVD

SEQ ID NO: 1000 - protein, 9 aa, Betula pendula
KERVDEVDH

SEQ ID NO: 1001 - protein, 9 aa, Betula pendula
NFKYSYSMI

SEQ ID NO: 1002 - protein, 9 aa, Betula pendula
FKYSYSMIE

SEQ ID NO: 1003 - protein, 9 aa, Betula pendula
KYSYSMIEG

SEQ ID NO: 1004 - protein, 9 aa, Betula pendula
YSYSMIEGG

SEQ ID NO: 1005 - protein, 9 aa, Betula pendula
SYSMIEGGA

SEQ ID NO: 1006 - protein, 9 aa, Betula pendula
YSMIEGGAL

SEQ ID NO: 1007 - protein, 9 aa, Betula pendula
SMIEGGALG

SEQ ID NO: 1008 - protein, 9 aa, Betula pendula
ALLRAVESY

SEQ ID NO: 1009 - protein, 9 aa, Betula pendula
LLRAVESYL

SEQ ID NO: 1010 - protein, 9 aa, Betula pendula
LRAVESYLL

SEQ ID NO: 1011 - protein, 9 aa, Betula pendula
RAVESYLLA

SEQ ID NO: 1012 - protein, 9 aa, Betula pendula
AVESYLLAH

SEQ ID NO: 1013 - protein, 9 aa, Betula pendula
VESYLLAHS

SEQ ID NO: 1014 - protein, 9 aa, Betula pendula
ESYLLAHSD

SEQ ID NO: 1015 - protein, 9 aa, Betula pendula
SYLLAHSDA

SEQ ID NO: 1016 - protein, 9 aa, Betula pendula
YLLAHSDAY

SEQ ID NO: 1017 - protein, 9 aa, Phleum pratense
GDEQKLRSA

SEQ ID NO: 1018 - protein, 9 aa, Phleum pratense
DEQKLRSAG

SEQ ID NO: 1019 - protein, 9 aa, Phleum pratense
EQKLRSAGE

SEQ ID NO: 1020 - protein, 9 aa, Phleum pratense
QKLRSAGEL

SEQ ID NO: 1021 - protein, 9 aa, Phleum pratense
KLRSAGELE

SEQ ID NO: 1022 - protein, 9 aa, Phleum pratense
LRSAGELEL

SEQ ID NO: 1023 - protein, 9 aa, Phleum pratense
RSAGELELQ

SEQ ID NO: 1024 - protein, 9 aa, Phleum pratense
NYLALLVKY

SEQ ID NO: 1025 - protein, 9 aa, Phleum pratense
YLALLVKYV

SEQ ID NO: 1026 - protein, 9 aa, Phleum pratense
LALLVKYVN

SEQ ID NO: 1027 - protein, 9 aa, Phleum pratense
ALLVKYVNG

SEQ ID NO: 1028 - protein, 9 aa, Phleum pratense
SWGAIWRID

SEQ ID NO: 1029 - protein, 9 aa, Phleum pratense
WGAIWRIDT

SEQ ID NO: 1030 - protein, 9 aa, Phleum pratense
GAIWRIDTP

SEQ ID NO: 1031 - protein, 9 aa, Phleum pratense
AIWRIDTPD

SEQ ID NO: 1032 - protein, 9 aa, Phleum pratense
IWRIDTPDK

SEQ ID NO: 1033 - protein, 9 aa, Phleum pratense
WRIDTPDKL

SEQ ID NO: 1034 - protein, 9 aa, Phleum pratense
RIDTPDKLT

SEQ ID NO: 1035 - protein, 9 aa, Phleum pratense
GDEQKLRSA

SEQ ID NO: 1036 - protein, 9 aa, Phleum pratense
DEQKLRSAG

SEQ ID NO: 1037 - protein, 9 aa, Phleum pratense
EQKLRSAGE

SEQ ID NO: 1038 - protein, 9 aa, Phleum pratense
QKLRSAGEL

SEQ ID NO: 1039 - protein, 9 aa, Phleum pratense
KLRSAGELE

SEQ ID NO: 1040 - protein, 9 aa, Phleum pratense
LRSAGELEL

SEQ ID NO: 1041 - protein, 9 aa, Phleum pratense
KINAGFKAA

SEQ ID NO: 1042 - protein, 9 aa, Phleum pratense
INAGFKAAL

SEQ ID NO: 1043 - protein, 9 aa, Phleum pratense
NAGFKAALA

SEQ ID NO: 1044 - protein, 9 aa, Phleum pratense
AGFKAALAA

SEQ ID NO: 1045 - protein, 9 aa, Phleum pratense
GFKAALAAA

SEQ ID NO: 1046 - protein, 9 aa, Phleum pratense
FKAALAAAA

SEQ ID NO: 1047 - protein, 9 aa, Phleum pratense
KAALAAAAG

SEQ ID NO: 1048 - protein, 9 aa, Phleum pratense
AALAAAAGV

SEQ ID NO: 1049 - protein, 9 aa, Phleum pratense
ALAAAAGVQ

SEQ ID NO: 1050 - protein, 9 aa, Phleum pratense
KYRTFVATF

SEQ ID NO: 1051 - protein, 9 aa, Phleum pratense
YRTFVATFG

SEQ ID NO: 1052 - protein, 9 aa, Phleum pratense
RTFVATFGA

SEQ ID NO: 1053 - protein, 9 aa, Phleum pratense
TFVATFGAA

SEQ ID NO: 1054 - protein, 9 aa, Phleum pratense
FVATFGAAS

SEQ ID NO: 1055 - protein, 9 aa, Phleum pratense
VATFGAASN

SEQ ID NO: 1056 - protein, 9 aa, Phleum pratense
TSKLDAAYK

SEQ ID NO: 1057 - protein, 9 aa, Phleum pratense
SKLDAAYKL

SEQ ID NO: 1058 - protein, 9 aa, Phleum pratense
KLDAAYKLA

SEQ ID NO: 1059 - protein, 9 aa, Phleum pratense
LDAAYKLAY

SEQ ID NO: 1060 - protein, 9 aa, Phleum pratense
DAAYKLAYK

SEQ ID NO: 1061 - protein, 9 aa, Phleum pratense
AAYKLAYKT

SEQ ID NO: 1062 - protein, 9 aa, Phleum pratense
AYKLAYKTA

SEQ ID NO: 1063 - protein, 9 aa, Phleum pratense
YKLAYKTAE

SEQ ID NO: 1064 - protein, 9 aa, Phleum pratense
KLAYKTAEG

SEQ ID NO: 1065 - protein, 9 aa, Phleum pratense
LAYKTAEGA

SEQ ID NO: 1066 - protein, 9 aa, Phleum pratense
KVDAAFKVA

SEQ ID NO: 1067 - protein, 9 aa, Phleum pratense
VDAAFKVAA

SEQ ID NO: 1068 - protein, 9 aa, Phleum pratense
DAAFKVAAT

SEQ ID NO: 1069 - protein, 9 aa, Phleum pratense
AAFKVAATA

SEQ ID NO: 1070 - protein, 9 aa, Phleum pratense
AFKVAATAA

SEQ ID NO: 1071 - protein, 9 aa, Phleum pratense
FKVAATAAN

SEQ ID NO: 1072 - protein, 8 aa, Phleum pratense
KVAATAAN

SEQ ID NO: 1073 - protein, 9 aa, Phleum pratense
VAATAANAA

SEQ ID NO: 1074 - protein, 9 aa, Phleum pratense
AATAANAAP

SEQ ID NO: 1075 - protein, 9 aa, Phleum pratense
SYKFIPALE

SEQ ID NO: 1076 - protein, 9 aa, Phleum pratense
YKFIPALEA

SEQ ID NO: 1077 - protein, 9 aa, Phleum pratense
KFIPALEAA

SEQ ID NO: 1078 - protein, 9 aa, Phleum pratense
FIPALEAAV

SEQ ID NO: 1079 - protein, 9 aa, Phleum pratense
IPALEAAVK

SEQ ID NO: 1080 - protein, 9 aa, Phleum pratense
PALEAAVKQ

SEQ ID NO: 1081 - protein, 9 aa, Phleum pratense
ALEAAVKQA

SEQ ID NO: 1082 - protein, 9 aa, Phleum pratense
LEAAVKQAY

SEQ ID NO: 1083 - protein, 9 aa, Phleum pratense
EAAVKQAYA

SEQ ID NO: 1084 - protein, 9 aa, Phleum pratense
KYTVFETAL

SEQ ID NO: 1085 - protein, 9 aa, Phleum pratense
YTVFETALK

SEQ ID NO: 1086 - protein, 9 aa, Phleum pratense
TVFETALKK

SEQ ID NO: 1087 - protein, 9 aa, Phleum pratense
VFETALKKA

SEQ ID NO: 1088 - protein, 9 aa, Phleum pratense
FETALKKAI

SEQ ID NO: 1089 - protein, 9 aa, Phleum pratense
ETALKKAIT

SEQ ID NO: 1090 - protein, 9 aa, Phleum pratense
TALKKAITA

SEQ ID NO: 1091 - protein, 9 aa, Phleum pratense
ALKKAITAM

SEQ ID NO: 1092 - protein, 9 aa, Phleum pratense
LKKAITAMS

SEQ ID NO: 1093 - protein, 9 aa, Phleum pratense
KKAITAMSE

SEQ ID NO: 1094 - protein, 9 aa, Alternaria alternata
QLLMLSAKR

SEQ ID NO: 1095 - protein, 9 aa, Alternaria alternata
LLMLSAKRM

SEQ ID NO: 1096 - protein, 9 aa, Alternaria alternata
LMLSAKRMK

SEQ ID NO: 1097 - protein, 9 aa, Alternaria alternata
MLSAKRMKV

SEQ ID NO: 1098 - protein, 9 aa, Alternaria alternata
LSAKRMKVA

SEQ ID NO: 1099 - protein, 9 aa, Alternaria alternata
SAKRMKVAF

SEQ ID NO: 1100 - protein, 9 aa, Alternaria alternata
AKRMKVAFK

SEQ ID NO: 1101 - protein, 9 aa, Alternaria alternata
KRMKVAFKL

SEQ ID NO: 1102 - protein, 9 aa, Alternaria alternata
RMKVAFKLD

SEQ ID NO: 1103 - protein, 9 aa, Alternaria alternata
MKVAFKLDI

SEQ ID NO: 1104 - protein, 9 aa, Alternaria alternata
KVAFKLDIE

SEQ ID NO: 1105 - protein, 9 aa, Alternaria alternata
GFKRCLQFT

SEQ ID NO: 1106 - protein, 9 aa, Alternaria alternata
FKRCLQFTL

SEQ ID NO: 1107 - protein, 9 aa, Alternaria alternata
KRCLQFTLY

SEQ ID NO: 1108 - protein, 9 aa, Alternaria alternata
RCLQFTLYR

SEQ ID NO: 1109 - protein, 9 aa, Alternaria alternata
CLQFTLYRP

SEQ ID NO: 1110 - protein, 9 aa, Alternaria alternata
LQFTLYRPR

SEQ ID NO: 1111 - protein, 9 aa, Alternaria alternata
QFTLYRPRD

SEQ ID NO: 1112 - protein, 9 aa, Alternaria alternata
FTLYRPRDL

SEQ ID NO: 1113 - protein, 9 aa, Alternaria alternata
TLYRPRDLL

SEQ ID NO: 1114 - protein, 9 aa, Alternaria alternata
LYRPRDLLS

SEQ ID NO: 1115 - protein, 9 aa, Alternaria alternata
YRPRDLLSL

SEQ ID NO: 1116 - protein, 9 aa, Alternaria alternata
RPRDLLSLL

SEQ ID NO: 1117 - protein, 9 aa, Alternaria alternata
TYYNSLGFN

SEQ ID NO: 1118 - protein, 9 aa, Alternaria alternata
YYNSLGFNI

SEQ ID NO: 1119 - protein, 9 aa, Alternaria alternata
YNSLGFNIK

SEQ ID NO: 1120 - protein, 9 aa, Alternaria alternata
NSLGFNIKA

SEQ ID NO: 1121 - protein, 9 aa, Alternaria alternata
SLGFNIKAT

SEQ ID NO: 1122 - protein, 9 aa, Alternaria alternata
LGFNIKATN

SEQ ID NO: 1123 - protein, 9 aa, Alternaria alternata
GFNIKATNG

SEQ ID NO: 1124 - protein, 9 aa, Alternaria alternata
FNIKATNGG

SEQ ID NO: 1125 - protein, 9 aa, Alternaria alternata
NIKATNGGT

SEQ ID NO: 1126 - protein, 9 aa, Alternaria alternata
IKATNGGTL

SEQ ID NO: 1127 - protein, 9 aa, Alternaria alternata
KATNGGTLD

SEQ ID NO: 1128 - protein, 9 aa, Alternaria alternata
DITYVATAT

SEQ ID NO: 1129 - protein, 9 aa, Alternaria alternata
ITYVATATL

SEQ ID NO: 1130 - protein, 9 aa, Alternaria alternata
TYVATATLP

SEQ ID NO: 1131 - protein, 9 aa, Alternaria alternata
YVATATLPN

SEQ ID NO: 1132 - protein, 9 aa, Alternaria alternata
VATATLPNY

SEQ ID NO: 1133 - protein, 9 aa, Alternaria alternata
ATATLPNYC

SEQ ID NO: 1134 - protein, 9 aa, Alternaria alternata
AYITLVTLP

SEQ ID NO: 1135 - protein, 9 aa, Alternaria alternata
YITLVTLPK

SEQ ID NO: 1136 - protein, 9 aa, Alternaria alternata
ITLVTLPKS

SEQ ID NO: 1137 - protein, 9 aa, Alternaria alternata
TLVTLPKSS

SEQ ID NO: 1138 - protein, 9 aa, Alternaria alternata
EVYQKLKAL

SEQ ID NO: 1139 - protein, 9 aa, Alternaria alternata
VYQKLKALA

SEQ ID NO: 1140 - protein, 9 aa, Alternaria alternata
YQKLKALAK

SEQ ID NO: 1141 - protein, 9 aa, Alternaria alternata
QKLKALAKK

SEQ ID NO: 1142 - protein, 9 aa, Alternaria alternata
KLKALAKKT

SEQ ID NO: 1143 - protein, 9 aa, Alternaria alternata
LKALAKKTY

SEQ ID NO: 1144 - protein, 9 aa, Alternaria alternata
KALAKKTYG

SEQ ID NO: 1145 - protein, 9 aa, Alternaria alternata
GYTGKIKIA

SEQ ID NO: 1146 - protein, 9 aa, Alternaria alternata
YTGKIKIAM

SEQ ID NO: 1147 - protein, 9 aa, Alternaria alternata
TGKIKIAMD

SEQ ID NO: 1148 - protein, 9 aa, Alternaria alternata
GKIKIAMDV

SEQ ID NO: 1149 - protein, 9 aa, Alternaria alternata
KIKIAMDVA

SEQ ID NO: 1150 - protein, 9 aa, Alternaria alternata
IKIAMDVAS

SEQ ID NO: 1151 - protein, 9 aa, Alternaria alternata
KIAMDVASS

SEQ ID NO: 1152 - protein, 9 aa, Alternaria alternata
IAMDVASSE

SEQ ID NO: 1153 - protein, 9 aa, Alternaria alternata
AMDVASSEF

SEQ ID NO: 1154 - protein, 9 aa, Alternaria alternata
AFGAGWGVM

SEQ ID NO: 1155 - protein, 9 aa, Alternaria alternata
FGAGWGVMV

SEQ ID NO: 1156 - protein, 9 aa, Alternaria alternata
GAGWGVMVS

SEQ ID NO: 1157 - protein, 9 aa, Alternaria alternata
AGWGVMVSH

SEQ ID NO: 1158 - protein, 9 aa, Alternaria alternata
GWGVMVSHR

SEQ ID NO: 1159 - protein, 9 aa, Alternaria alternata
WGVMVSHRS

SEQ ID NO: 1160 - protein, 9 aa, Alternaria alternata
GVMVSHRSG

SEQ ID NO: 1161 - protein, 9 aa, Alternaria alternata
VMVSHRSGE

SEQ ID NO: 1162 - protein, 9 aa, Alternaria alternata
MVSHRSGET

SEQ ID NO: 1163 - protein, 9 aa, Alternaria alternata
VSHRSGETE